(19) United States
(12) Patent Application Publication
(10) Pub. No.: US 2026/0251643 A1
(43) Pub. Date: Aug. 27, 2026

(54) METHODS AND DEVICES FOR DETECTING BIOLOGICAL ANALYTES USING TIME-RESOLVED OPTICAL REPORTERS

(71) Applicant: Siomyx, Inc., Mountain View, CA (US)

(72) Inventor: Arjang HASSIBI, Palo Alto, CA (US)

(21) Appl. No.: 19/348,178

(22) Filed: Oct. 2, 2025

Related U.S. Application Data
(63) Continuation of application No. PCT/US2024/023418, filed on Apr. 5, 2024.
(60) Provisional application No. 63/457,247, filed on Apr. 5, 2023.

(51) Int. Cl. G01N 33/543; G01N 21/17; G01N 33/542; G01N 33/58

(52) U.S. Cl. CPC G01N 33/54366; G01N 21/17; G01N 33/542; G01N 33/582; G01N 2021/1791

(57) ABSTRACT

The present disclosure provides methods, devices, and systems to detect, in real-time, the presence, abundance and/or interactions of one or more biological analytes in an aqueous medium. Using time-resolved optical methods, the system may be subjected to a short excitation pulse photon flux and the detection may include measuring and subsequently analyzing the temporal optical response of system in the presence and absence of the analytes. Methods may include using the devices and systems described herein for analyte detection.

### CROSS-REFERENCE

This application is a continuation of International Application No. PCT/US2024/023418, filed Apr. 5, 2024, which claims the benefit of U.S. Provisional Application No. 63/457,247 filed Apr. 5, 2023, each of which is incorporated by reference herein in their entirety.

### SEQUENCE LISTING

The present application contains a Sequence Listing which has been submitted electronically in XML format and is hereby incorporated by reference in its entirety. The XML copy, created on Oct. 2, 2025, is named 63452-703_301_SL.xml and is 4,625 bytes in size.

### BACKGROUND

Real-time detection of biomolecular and chemical analytes is a key method to non-invasively analyze the dynamics of biological and biochemical systems. In real-time methods, the presence and/or concentration and/or reactivity of an analyte is estimated while the analyte interacts with other biomolecular structures in the system. The dynamics of the system are therefore measurable in real-time, hence, advantageous. This is in contrast with the end-point detection methods, in which the biomolecular reaction is initially stopped, and analysis is performed afterwards which provides only a snapshot of dynamics of the system when analytes are unable to freely interact anymore.

### SUMMARY

Recognized herein is a need for improved systems, devices, and methods for identification and quantification of analytes. Such systems may include detection methods integrated with semiconductor-based optical sensor devices to identify and quantify analytes, for example, in an aqueous sample. Methods and devices described herein may provide real-time detection of target analytes, including biological molecules of interest.

An aspect of the present disclosure provides a real-time detection system for at least one analyte, comprising: (a) a reaction chamber comprising a plurality of capturing probes immobilized at an independently addressable location on a surface of the reaction chamber, wherein each of the plurality of capturing probes comprises at least one reporter molecule, and wherein the plurality of capturing probes comprise a capturing probe; and (b) a sensor array comprising a sensor in optical communication with the independently addressable location on the surface, wherein the sensor is configured to detect at least one signal from the independently addressable location in real-time, wherein the at least one signal is time-resolved, thereby allowing determination of a fluorescence emission lifetime of the at least one signal.

In some embodiments, the real-time detection system further comprises: a light source configured to synchronize with the sensor array and emit a pulse of excitation energy when turned on, wherein the at least one signal is collected after the light source is turned off, wherein said at least one analyte comprises an analyte. In some embodiments, the at least one reporter molecule is a fluorescence reporter, wherein, when the capturing probe is not bound with the analyte, the fluorescence reporter is configured to emit a first fluorescence comprising a first time-resolved signal with a first fluorescence emission lifetime upon excitation by the pulse of excitation energy. In some embodiments, when the capturing probe is bound with the analyte, the fluorescence reporter is configured to emit a second time-resolved signal with a second fluorescence emission lifetime upon excitation by the pulse of excitation energy, and wherein the second fluorescence lifetime is different from the first fluorescence lifetime. In some embodiments, the fluorescence reporter is an energy donor and forms a donor-quencher pair with a non-radiating quencher, wherein, when the fluorescence reporter is brought into a proximity of the non-radiating quencher, the first fluorescence is quenched by the non-radiating quencher. In some embodiments, the analyte comprises the non-radiating quencher, wherein the capturing probe is configured to capture the analyte and form a duplex, wherein the duplex brings the energy donor and the non-radiating quencher into the proximity. In some embodiments, the at least one reporter molecule is a fluorescence reporter, wherein the fluorescence reporter is a donor of a fluorescence resonance energy transfer (FRET) system, wherein, when the capturing probe is not bound with the analyte, the fluorescence reporter is configured to emit a first fluorescence comprising a first time-resolved signal with a first fluorescence emission lifetime upon excitation by the pulse of excitation energy. In some embodiments, the FRET system comprises an acceptor, wherein, when the acceptor is brought within a vicinity of the donor to form the FRET system, the acceptor is configured to emit a second fluorescence comprising a second time-resolved signal with a second fluorescence emission lifetime upon excitation by the first fluorescence, the second fluorescence emission lifetime is different from the first fluorescence lifetime. In some embodiments, the analyte comprises the acceptor. In some embodiments, the capturing probe is configured to capture the analyte and form a duplex, the duplex brings the donor and the acceptor into the vicinity. In some embodiments, the capturing probe is configured to capture the analyte and form a duplex, the duplex intercalates an intercalator comprising the acceptor. In some embodiments, the duplex brings the donor and the acceptor into the vicinity. In some embodiments, the at least one reporter molecule comprises a fluorescence resonance energy transfer (FRET) system comprising a donor and an acceptor, the donor is configured to emit a first fluorescence comprising a first time-resolved signal with a first fluorescence emission lifetime upon excitation by the pulse of excitation energy, wherein, when the acceptor is brought within a vicinity of the donor to form the FRET system, the acceptor is configured to emit a second fluorescence comprising a second time-resolved signal with a second fluorescence emission lifetime upon excitation by the first fluorescence, the second fluorescence emission lifetime is different from the first fluorescence lifetime. In some embodiments, when the capturing probe is bound with the analyte, the capturing probe is in a first configuration which emits the first time-resolved signal. In some embodiments, when the capturing probe is not bound with the analyte, the capturing probe is in a second configuration which emits the second time-resolved signal. In some embodiments, the at least one reporter molecule comprises an energy donor and a non-radiating quencher, the donor is configured to emit a first fluorescence comprising a first time-resolved signal with a first fluorescence emission lifetime upon excitation by the pulse of excitation energy, the first fluorescence is quenched by the non-radiating quencher when the energy donor is brought into a proximity of the non-radiating quencher. In some embodiments, when the capturing probe is not bound with the analyte, the capturing probe is in a first configuration which brings the energy donor and the non-radiating quencher into the proximity. In some embodiments, when the capturing probe is bound with the analyte, the capturing probe is in a second configuration which emits the first time-resolved signal. In some embodiments, a ratio of an overall error/inaccuracy of timing measurement to the first fluorescence emission lifetime or the second fluorescence emission lifetime is no more than 0.01. In some embodiments, the ratio is no more than 1×10⁻⁶. In some embodiments, the excitation energy comprises an excitation amplitude, a first resolution of a measurement of the excitation amplitude is no less than 8 bits. In some embodiments, the first resolution is no less than 12 bits. In some embodiments, the first fluorescence comprises an emission amplitude, a second resolution of a measurement of the emission amplitude is no less than 14 bits. In some embodiments, the second resolution is no less than 20 bits. In some embodiments, further comprises a focal plane imager with an electrical shutter, the focal plane imager is configured to take time-gated images of the surface. In some embodiments, the focal plane imager is a camera or a microscope. In some embodiments, the time-gated images comprise no less than 10 imaging pixels per the independently addressable location. In some embodiments, the time-gated images comprise no less than 104 imaging pixels per the independently addressable location. In some embodiments, the sensor array is an integrated biosensor array, the integrated biosensor array is within a proximity of the surface and in optical communication with the surface. In some embodiments, further comprises at least one control probe immobilized on the surface. In some embodiments, the at least one control probe is: (i) a donor control probe comprising a fluorescence donor, the donor control probe is configured not to capture or interact with the analyte; or (ii) an acceptor control probe comprising a fluorescence resonance energy transfer (FRET) control system comprising a FRET donor and a FRET acceptor, the FRET control system is configured to emit a FRET signal from the FRET acceptor, the acceptor control probe is configured not to capture or interact with the analyte; or (iii) a blank probe configured not to emit a fluorescence signal; or (iv) a combination thereof. In some embodiments, the sensor comprises: (i) a first optical transducer in optical communication with the surface; (ii) a second optical transducer disposed adjacent to the first optical transducer; and (iii) an optical cover disposed over the second optical transducer, the first optical transducer is configured to collect the at least one signal. In some embodiments, the at least one signal comprises: a first optical signal from the surface generated upon exposure of the surface to the pulse of excitation energy, the first optical signal is collected by the first optical transducer and is converted to a first electrical signal; and a second optical signal, the second optical signal is collected by the second optical transducer and is converted to a second electrical signal. In some embodiments, the sensor further comprises a current switch operably connected to the first optical transducer and the second optical transducer, the current switch is configured to: (i) divert the first and second electrical signals to a low gain detection path during a first time period when the light source is on; and (i) divert the first and second electrical signals to a high gain detection path during a second time period when the light source is off. In some embodiments, the first optical transducer and the second optical transducer are separated by a distance about 100 nanometers (nm) to about 1 millimeter (mm). In some embodiments, the first optical transducer is a first photodiode, a first photogate, or a first photo-resistive device. In some embodiments, the second optical transducer is a second photodiode, a second photogate, or a second photo-resistive device. In some embodiments, the first optical transducer is a first photodiode, and the second optical transducer is a second photodiode. In some embodiments, the optical cover is configured to reduce an amount of photons emitted by the light source from contacting the second optical transducer as compared to an optical transducer without the optical cover. In some embodiments, the optical cover comprises a metal. In some embodiments, the sensor array does not include an emission filter. In some embodiments, further comprises one or more optical isolators disposed adjacent to the first and/or second optical transducers, the one or more optical isolators are configured to direct photons to the first or second optical transducers. In some embodiments, the sensor array does not include an optical filter.

Another aspect of the present disclosure provides a method for time-resolved fluorescence detection of a presence or absence of an analyte in a solution, the method comprising: (a) directing the solution to a reaction chamber of a biochip synchronized with a light source operably coupled to the biochip, the reaction chamber comprises a surface comprising a plurality of capturing probes immobilized at an independently addressable location on the surface and in fluid contact with the solution, each of the plurality of capturing probes comprises at least one reporter molecule, and the plurality of capturing probes comprises a capturing probe; (b) exciting the at least one reporter molecule by the light source for a first time period; (c) turning off the light source after the first time period; (d) recording a time-resolved fluorescence emission signal from the surface over a second time period while the light source is turned off; and (e) determining a fluorescence emission lifetime of the time-resolved fluorescence emission signal.

In some embodiments, the at least one reporter molecule is a fluorescence reporter, wherein, in (d) when the capturing probe is not bound with the analyte, the fluorescence reporter emits a first fluorescence comprising a first time-resolved signal with a first fluorescence emission lifetime upon excitation by the light source. In some embodiments, in (d) when the capturing probe is bound with the analyte, the fluorescence reporter emits a second fluorescence comprising a second time-resolved signal with a second fluorescence emission lifetime upon excitation by the light source, and the second fluorescence emission lifetime is different from the first fluorescence emission lifetime. In some embodiments, the analyte comprises a non-radiating quencher, the fluorescence reporter is an energy donor and forms a donor-quencher pair with the non-radiating quencher when the fluorescence reporter is brought into a proximity of the non-radiating quencher. In some embodiments, the method further comprises: capturing the analyte by the capturing probe and forming a duplex, thereby bringing the energy donor and the non-radiating quencher into the proximity and quenching at least some of the first fluorescence. In some embodiments, the at least one reporter molecule is a fluorescence reporter, the fluorescence reporter is a donor of a fluorescence resonance energy transfer (FRET) system, the fluorescence reporter emits a first fluorescence comprising a first time-resolved signal with a first fluorescence emission lifetime upon excitation by the light source. In some embodiments, the FRET system comprises an acceptor, the acceptor emits a second fluorescence comprising a second time-resolved signal with a second fluorescence emission lifetime upon excitation by the first fluorescence when the acceptor is brought within a vicinity of the donor to form the FRET system, the second fluorescence lifetime is different from the first fluorescence lifetime. In some embodiments, the method further comprises: capturing the analyte by the capturing probe and forming a duplex, the analyte comprises the acceptor, the duplex brings the donor and the acceptor into the vicinity. In some embodiments, the method further comprises: capturing the analyte by the capturing probe and forming a duplex, the duplex intercalates an intercalator comprising the acceptor and brings the donor and the acceptor into the vicinity. In some embodiments, the at least one reporter molecule comprise a fluorescence resonance energy transfer (FRET) system comprising a donor and an acceptor, the donor, in a presence of the analyte, emits a first fluorescence comprising a first time-resolved signal with a first fluorescence emission lifetime upon excitation by the light source, the acceptor emits a second time-resolved signal with a second fluorescence emission lifetime upon excitation by the first fluorescence when the acceptor is brought in a vicinity of the donor to form the FRET system, the second fluorescence lifetime is different from the first fluorescence lifetime. In some embodiments, further comprises: capturing the analyte by the capturing probe and emitting the first fluorescence comprising the first time-resolved signal. In some embodiments, the method further comprises: emitting the second time-resolved signal in an absence of the analyte. In some embodiments, the at least one reporter molecule comprise an energy donor and a non-radiating quencher, the donor emits a first fluorescence comprising a first time-resolved signal with a first fluorescence emission lifetime upon excitation by the light source, the first fluorescence is quenched by the non-radiating quencher when the energy donor is brought into a proximity of the non-radiating quencher. In some embodiments, the method further comprises: capturing the analyte by the capturing probe and emitting the first fluorescence comprising the first time-resolved signal. In some embodiments, the method further comprises: quenching the first fluorescence by the non-radiating quencher in an absence of the analyte. In some embodiments, the method further comprises: monitoring a first ratio of an overall error/inaccuracy of timing measurement to the first fluorescence emission lifetime or the second fluorescence emission lifetime, the first ratio is no more than 0.01. In some embodiments, the ratio is no more than 1×10⁻⁶. In some embodiments, the method comprises: measuring a first resolution of a measurement of an excitation amplitude of an excitation energy emitted by the light source, the first resolution is no less than 8 bits. In some embodiments, the first resolution is no less than 12 bits. In some embodiments, the method further comprises: measuring a second resolution of a measurement of an emission amplitude of the first fluorescence, the second resolution is no less than 14 bits. In some embodiments, the second resolution is no less than 20 bits. In some embodiments, the recording in (d) comprises taking time-gated images of the surface by a focal plane imager with an electrical shutter. In some embodiments, the focal plane imager is a camera or a microscope. In some embodiments, the time-gated images comprises no less than 10 imaging pixels per the independently addressable location. In some embodiments, the time-gated images comprises no less than 104 imaging pixels per the independently addressable location. In some embodiments 44-68, the recording in (d) comprising recording the time-resolved fluorescence emission signal by an integrated biosensor array, the integrated biosensor array is within a proximity of the surface and in optical communication with the surface. In some embodiments, the integrated biosensor array comprises at least one electrical shutter, the at least one electrical shutters is in electronic communication with photosensors to record the fluorescence emission signal. In some embodiments, the determining in (e) comprises determining the fluorescence emission lifetime as a function of the first fluorescence emission lifetime and/or the second fluorescence emission lifetime. In some embodiments, the second fluorescence emission lifetime is greater than the first fluorescence emission lifetime. In some embodiments, the method further comprises: prior to (a), (a0) in an absence of the analyte in the reaction chamber, exciting the at least one reporter molecule by the light source for a third time period; (a1) turning off the light source after the third time period; (a2) recording an additional time-resolved fluorescence emission signal from the surface over a fourth time period while the light source is turned off; and (a3) determining an additional fluorescence emission lifetime of the additional time-resolved fluorescence emission signal. In some embodiments, the method further comprises: calculating the first fluorescence emission lifetime or the second fluorescence emission lifetime based on at least the additional fluorescence emission lifetime.

Another aspect of the present disclosure provides a method for time-resolved fluorescence assaying a presence or absence of an analyte in a solution, comprising: (a) directing the solution containing or suspected of containing the analyte to a reaction chamber of a biochip synchronized with a light source operably coupled to the biochip, the reaction chamber comprises a surface comprising a plurality of capturing probes immobilized at an independently addressable location on the surface and in fluid contact with the solution, each of the plurality of capturing probes is configured to selectively couple to the analyte, each of the plurality of capturing probes comprises a donor reporter molecule of a fluorescence resonance energy transfer (FRET) system, the donor reporter molecule emits a first fluorescence comprising a first time-resolved signal with a first fluorescence emission lifetime upon excitation by the light source, the plurality of capturing probes comprises a capturing probe; (b) bringing the reaction chamber under conditions sufficient to permit the capturing probe to selectively couple to the analyte, the analyte comprises an acceptor reporter molecule of the FRET system, the acceptor reporter molecule emits a second fluorescence comprising a second time-resolved signal with a second fluorescence emission lifetime upon excitation by the first fluorescence when the acceptor reporter molecule is brought within a vicinity of the donor reporter molecule to form the FRET system, and the first lifetime is longer than the second lifetime; (c) exciting the at least one reporter molecule by the light source for a first time period; (d) turning off the light source after the first time period; (e) recording a third time-resolved fluorescence emission signal from the independently addressable location on the surface at a first temperature over a second time period while the light source is turned off; (f) recording a fourth time-resolved fluorescence emission signal from the independently addressable location on the surface at a second temperature over a third time period while the light source is turned off, the first temperature is different from the second temperature; and (g) analyzing at least the third time-resolved fluorescence emission signal and the fourth time-resolved fluorescence emission signal, thereby assaying the presence of the analyte in the solution.

In some embodiments, the method further comprises, after (e) and before (f): (i) exciting the at least one reporter molecule by the light source for a fourth time period; and (ii) turning off the light source after the fourth time period. In some embodiments, the third time-resolved fluorescence emission signal is measured against a first background signal at the first temperature, and the fourth time-resolved fluorescence emission signal is measured against a second background signal at the second temperature. In some embodiments, the method further comprises: determining a single nucleotide polymorphism (SNP) in a sequence of the analyte using at least the third time-resolved fluorescence emission signal and the fourth time-resolved fluorescence emission signal. In some embodiments, (e) and (f) are performed while the solution is in contact with the surface. In some embodiments, the method further comprises using at least the third time-resolved fluorescence emission signal and the fourth time-resolved fluorescence emission signal to assess a thermodynamic characteristic of an interaction between the capturing probe and the analyte. In some embodiments, the method further comprises, prior to (a), conducting a nucleic acid amplification reaction under conditions sufficient to yield the analyte in the solution. In some embodiments, the solution containing or suspected of containing another analyte, the surface comprising a plurality of additional capturing probes immobilized at another independently addressable location on the surface and in fluid contact with the solution, each of the plurality of additional capturing probes is configured to selectively couple to the another analyte. In some embodiments, each of the plurality of additional capturing probes comprises another donor reporter molecule of another FRET system, and the another analyte comprises another acceptor reporter molecule of the another FRET system, the FRET system is different from the another FRET system. In some embodiments, the method further comprises, in (e), recording a fifth time-resolved fluorescence emission signal from the another independently addressable location on the surface at the first temperature over the second time period while the light source is turned off. In some embodiments, the method further comprises, in (f), recording a sixth time-resolved fluorescence emission signal from the another independently addressable location on the surface at the second temperature over the third time period while the light source is turned off. In some embodiments, the acceptor reporter molecule is not a non-radiating quencher.

Another aspect of the present disclosure provides a method for assaying at least one analyte, comprising: (a) subjecting a reaction mixture to a nucleic acid amplification reaction under conditions sufficient to yield at least one target nucleic acid molecule as an amplification product of the at least one analyte, the reaction mixture comprises (i) a sample containing or suspected of containing the at least one analyte, (ii) at least one primer set, and (iii) a polymerizing enzyme, a first primer of a first primer set of the at least one primer set has sequence complementarity with a first analyte of the at least one analyte, a first target nucleic acid molecule of the at least one target nucleic acid molecule is the amplification product of the first analyte, the first target nucleic acid molecule comprises the first primer; (b) bringing the reaction mixture in contact with a reaction chamber of a biochip synchronized with a light source operably coupled to the biochip, the reaction chamber comprises a surface comprising a plurality of capturing probes immobilized at an independently addressable location on the surface and in fluid contact with the reaction mixture, each of the plurality of capturing probes is configured to selectively couple to either the first primer or the first target nucleic acid molecule, each of the plurality of capturing probes comprises a donor reporter molecule, the donor reporter molecule emits a first time-resolved signal with a first fluorescence emission lifetime upon excitation by the light source in an absence of the first primer, the plurality of capturing probes comprises a first capturing probe; (c) after a first amplification cycle of the nucleic acid amplification reaction, exciting the donor reporter molecule by the light source for a first time period, then turning off the light source after the first time period; (d) recording a first time-resolved fluorescence emission signal from the surface over a second time period while the light source is turned off; (e) after a second amplification cycle of the nucleic acid amplification, recording a second time-resolved fluorescence emission signal from the surface over a third time period while the light source is turned off, the first amplification cycle is before the second amplification cycle; (f) determining a second fluorescence emission lifetime of the first time-resolved fluorescence emission signal and a third fluorescence emission lifetime of the second time-resolved fluorescence emission signal, thereby assaying the at least one analyte in the sample.

In some embodiments, the assaying the at least one analyte in (f) comprises determining a first property of the first analyte in the sample. In some embodiments, the first property is at least one of an initial concentration of the first analyte in the sample, a binding rate of the first primer with the first capturing probe, or a presence or absence of the first analyte in the sample. In some embodiments, the method further comprises correlating the first time-resolved fluorescence emission signal and the second time-resolved fluorescence emission signal with the initial concentration of the first analyte in the sample by analyzing the binding rate. In some embodiments, each of the plurality of capturing probes is configured to selectively couple the first primer but not the first target nucleic acid molecule. In some embodiments, each of the plurality of capturing probes is configured to selectively couple the first target nucleic acid molecule but not the first primer. In some embodiments, the donor reporter molecule is attached at or near an 3′-end of each of the plurality of capturing probes. In some embodiments, each of the plurality of capturing probes is immobilized to the surface via a 5′-end. In some embodiments, each of the plurality of capturing probes is immobilized to the surface via a 3′-end. In some embodiments, the at least one analyte comprises a second analyte, the at least one target nucleic acid molecule comprises a second target nucleic acid molecule, the second target nucleic acid molecule is an amplification product of the second analyte in the nucleic acid amplification using a second primer of a second primer set of the at least one primer set, the surface comprising another plurality of capturing probes immobilized at another independently addressable location on the surface, each of the another plurality of capturing probes is configured to selectively couple to the second primer, the another plurality of capturing probes comprises a second capturing probe comprising another donor reporter molecule. In some embodiments, the method further comprises: determining a second property of the second analyte in the sample, the second property is at least one of an initial concentration of the second analyte in the sample, a binding rate of the second primer with the second capturing probe, or a presence or absence of the second analyte in the sample. In some embodiments, the donor reporter molecule emits a second time-resolved signal with a fourth fluorescence emission lifetime when the first capturing probe couples to the first primer and upon excitation by the light source. In some embodiments, the first time-resolved fluorescence emission signal and the second time-resolved fluorescence emission signal are indicative of an interaction between the donor reporter molecule with an acceptor reporter molecule. In some embodiments, the acceptor reporter molecule is coupled to the first primer. In some embodiments, the acceptor reporter molecule is coupled at or near a 5′-end of the first primer. In some embodiments, the acceptor reporter molecule is coupled near a 3′-end of the first primer. In some embodiments, the interaction is a fluorescence resonance energy transfer (FRET). In some embodiments, the interaction is a non-radiating quenching. In some embodiments, the fourth fluorescence emission lifetime is different from the first fluorescence emission lifetime. In some embodiments, the assaying the at least one analyte in (f) further comprises determining (i) a first relationship of the second fluorescence emission lifetime with respect to the first fluorescence emission lifetime and the fourth fluorescence emission lifetime; and (ii) a second relationship of the third fluorescence emission lifetime with respect to the first fluorescence emission lifetime and the fourth fluorescence emission lifetime. In all the embodiments disclosed above, the first capturing probe is an oligonucleotide, and the first analyte is a nucleic acid molecule.

In all the above embodiments for various methods, the method further comprises: modulating the light source and emitting a pulse of excitation energy. In all the above embodiments for various methods, the method further comprises: calculating a percentage of capturing probes bound with the analyte or the first analyte relative to the plurality of capturing probes at the independently addressable location. In some embodiments, the calculating the percentage is based on at least the fluorescence emission lifetime, the first fluorescence emission lifetime, and/or the second fluorescence emission lifetime, and/or the third fluorescence emission lifetime, and/or the fourth fluorescence emission lifetime. In some embodiments, the method further comprises: determining a concentration of the analyte in the solution or the first analyte in the sample based on at least the percentage. In some embodiments, the biochip does not include an emission filter and/or an optical filter. In some embodiments, the biochip is the real-time detection system of any one of the embodiments disclosed herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A shows a diagram of an example real-time detection system; FIG. 1B shows another diagram of another example real-time detection system and how in real-time interactions and/or capturing of analytes can be used by the sensor array to create analyte-dependent outputs;

FIG. 2 shows a diagram of an example real-time detection method that can take advantage of fluorescence-labelled capturing probes and quencher labelled analytes. The capturing of analytes in this system may reduce the fluorescence emission signal which may be used as a quantitative indicator for analyte capturing by a fluorescence detection device;

FIG. 3 shows a diagram of an example real-time detection method that can take advantage of reduction-oxidation (redox) reporters embedded in molecular switches. The capturing of analytes in this system can reduce the electron transfer amplitude (or rate) which can be used as a quantitative indicator for analyte capturing by a cyclic voltammetry system or device;

FIG. 4A shows an example components of a time-resolved optical detection system;

FIG. 4B shows an example excitation waveform and an example emission waveform of a time-resolved optical detection system;

FIG. 5 shows a diagram of an example method of creating time-resolved optical signals in which capturing of the analyte can change the non-radiating relaxation paths of fluorescence reporter and increases the lifetime from τ to τ*;

FIG. 6 shows an example photon emission waveform of a biosensor that uses the method of FIG. 5;

FIG. 7 shows an example method of creating time-resolved optical signals in which capturing of a labeled analyte by a labeled probe forms a fluorescence resonance energy transfer (FRET) and switches the measured lifetime of the observed decaying signal from τ to τ*;

FIG. 8 shows an example photon emission waveform of a biosensor that uses the method of FIG. 7;

FIG. 9 shows another example method of creating time-resolved optical signals in which capturing of a labeled analyte which can form a duplex facilitating the attachment of intercalator fluorophores to create a fluorescence resonance energy transfer (FRET) system. This switches the measured lifetime of the observed decaying signal from τ to τ*;

FIG. 10 shows another example photon emission waveform of a biosensor that uses the method of FIG. 9;

FIG. 11 shows an example method of creating time-resolved optical signals in which capturing of a labeled analyte can quench the emission.

FIG. 12 shows an example photon emission waveform of a biosensor that uses the method of FIG. 11.

FIG. 13 shows an example method of creating time-resolved optical signals in which capturing of an analyte changes the state of a molecular switch that acts as the capturing probe. Once the analyte is captured, the measured lifetime of the observed decaying signal switches from τ* to τ.

FIG. 14 photon emission waveform of a biosensor that uses the method of FIG. 13;

FIG. 15 shows an example method of creating time-resolved optical signals in which capturing of an analyte can change the state of a molecular switch that acts as the capturing probe. Once the analyte is captured, the structure can emit a signal with a lifetime of T;

FIG. 16 shows example photon emission waveform of a biosensor that uses the method of FIG. 15;

FIG. 17 shows an example measurement and its timing diagram for a time-resolved biosensor;

FIG. 18A shows an example implementation category to create the sensor array using a focal plane imager; FIG. 18B shows an example implementation category to create an integrated biosensor array to act as the substrate for the capturing probes;

FIG. 19A shows an example capturing probe/spot; FIG. 19B shows an example donor control probe/spot; FIG. 19C shows an example acceptor control probe/spot; FIG. 19D shows an example blank probe/spot;

FIG. 20A shows an example real-time microarray system that can use time-resolved optical reporters to measure the reaction kinetics. The results are shown in FIG. 20B. FIGS. 20C-20D shows that the decaying signal from long lifetime fluorescence reporters can be recorded during the capturing process which then can be used to compute the forward reaction rate and ultimately estimate the analyte concentration;

FIG. 21A shows an example solid-phase melt curve analysis that uses time-resolved optical reporters to measure the stability of analyte-probe duplexes subject to the temperature being increased gradually. The results are shown in FIG. 21B. FIGS. 21C-21D show that the monotonically increasing signals can be measured as the indicator of analyte-probe duplex stability during the temperature ramp;

FIG. 22A shows an example of two different capturing probes (probe A and probe
B) capturing an analyte (either analyte A or analyte B) at capturing spot A and capturing spot B, respectively, followed by melt analysis of the formed complex, when the system is subject to a specific temperature profile as shown in FIG. 22B, which includes a thermal reset phase, followed by a capturing phase and a denaturing phase. FIG. 22C shows the expected signals for analyte A at the capturing spots A and B, respectively, including a kinetic capturing phase with rates that can be concentration-dependent and melt curves that can be a function of the bond (duplex) energies;

FIG. 23A shows an example differential melt curve analysis in which an unknown target is introduced to probes A (targeting wild-type analyte) and probes B (targeting mutant analyte) which have dissimilar sequences residing on capturing spots A and B, respectively. A specific temperature profile in FIG. 23B is then subjected to the real-time microarray for melt curve analysis. FIGS. 23C-23D show two possible results which may happen: When wild-type analyte is present, the capturing spot with wild-type probes shows a more stable duplex than the capturing spot with mutant type probes; and when the mutant analyte is present, the reverse happens;

FIG. 24 shows an example to detect polymerase chain reaction (PCR) amplicons in real time. Primers can be labeled with short lifetime fluorescence reporters (acceptors) to create labeled amplicons. Solid-phase capturing probes that include long lifetime fluorescence reporters (donors) can then be used to specifically capture the amplicons;

FIG. 25 shows a computer system that is programmed or otherwise configured to implement methods provided herein;

FIG. 26 shows a block diagram of an example sensor system using a fluorescent-based transduction method;

FIGS. 27A-27C show example differential photodiode transducers and circuit schematics; FIG. 27A shows an example differential photodiode and circuit schematic; FIG. 27B shows an example array of eight photodiode transducers and circuit schematic; and FIG. 27C shows an example array of three photodiode transducers and circuit schematic;

FIG. 28 shows an example current switch and timing diagram;

FIG. 29 shows an example current switch with stabilization switches;

FIG. 30 shows an example sensing array and pixel cross-section;

FIG. 31 shows an example block diagram and timing diagram of an example detection circuit;

FIG. 32 shows another example block diagram and timing diagram of another example detection circuit; and

FIG. 33 shows another example block diagram and timing diagram of another example detection circuit.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time analysis” or “real-time detection” as used herein generally refers to the detection and quantification of an analyte, such as a biological or biochemical molecular analyte, including but not limited to a deoxyribonucleic acid (DNA), a ribonucleic acid (RNA), or a small peptide, in a biological or biochemical system that allows the analyte to freely move, through diffusion and/or drift, and interact with other biological and chemical structures and biomolecules.

### Definitions

The term “real-time detection system” as used herein generally refers to a system that performs real-time analysis on an aqueous biological or biochemical sample that includes one or more analytes.

The term “fluorescent intercalator” or “intercalator” as used herein generally refers to a small molecule that reversibly binds to or inserts between bases of double-stranded nucleic acid (e.g., double-stranded DNA). The fluorescent intercalator emits a fluorescent signal when bound to the double-stranded nucleic acid and produces less or no signal when not bound to double-stranded nucleic acid. Examples of fluorescent DNA intercalators include, but are not limited to, SYBR® Green I, SYBR® Green II, SYBR® Gold, YOYO®, YO-PRO™, TOTO®, PicoGreen®, and EvaGreen®.

The term “quencher” or “non-radiating quencher” or “non-radiating optical quencher” as used herein generally refers to a compound (e.g., a small molecule dye) that quenches a signal emitted from a fluorescent compound, e.g., a fluorescent intercalator or a fluorescence reporter. In some embodiments, the quencher absorbs excitation energy from the fluorescent compound (e.g., fluorescent intercalator or fluorescent reporter) and dissipates the energy that is absorbed from the fluorescent compound as heat (not a new fluorescence signal).

The term “fluorescence-based detection” as used herein generally refers to a detection scheme that uses a wavelength-specific optical excitation light source to excite fluorophore constructs that may subsequently re-emit light in a different wavelength. A fluorescence detection device or instrument (e.g., fluorescence sensor) may measure the emission signal, which may represent the quantity of the fluorophore construct, in the presence of a much larger excitation signal.

The term “analyte,” as used herein, generally refers to a molecular species to be detected. Non-limiting examples include small molecules, such as organic compounds drugs, hormones, lipids, steroids, or metabolites; polynucleotides such as deoxyribonucleic acid (DNA) molecules, ribonucleic acid (RNA) molecules, and peptide nucleic acid (PNA) molecules; polypeptides such as proteins, peptides, antibodies, antigens, enzymes, and receptors; as well as tissues, organelles, and other receptor probes.

The term “probe” or “capture probe” or “capturing probe” may be used interchangeably and generally refers to a molecular species or other markers that can bind and/or interact to a specific analyte. Probes can comprise molecules and can be bound to the substrate, molecules, or other solid surface, directly or via a linker. Non-limiting examples of linkers include amino acids, polypeptides, nucleotides, oligonucleotides, and chemical linkers. A plurality of probes can be immobilized to a substrate, molecule or other solid surface and can be referred to as a probe array. A plurality of probes of a probe array may be arranged uniformly, for example as an arrangement of spots, or non-uniformity.

The term “reporter” or “reporter molecule” as used herein, generally refers to a molecular structure that can be attached to a molecule (e.g., an analyte or a probe) or be associated with a molecule or construct (e.g., a duplex of a double stranded nucleic acid) covalently or non-covalently, to permit detection of the molecule, distinguishable or traceable by providing a characteristic which may not be intrinsic to the analyte molecule. Examples of reports or reporter molecules or labels may include luminescent molecules (e.g., fluorophores), reduction-oxidation (redox) species, or enzymes. In some cases, labels may comprise fluorophores with long lifetimes, such as, for example, lanthanide chelates and transition metal chelates, which may be luminescent or phosphorescent.

The term “nucleotide,” as used herein, generally refers to a molecule that can serve as the monomer, or subunit, of a nucleic acid, such as deoxyribonucleic acid (DNA) or ribonucleic acid (RNA). A nucleotide may be a deoxynucleotide triphosphate (dNTP) or an analog thereof (e.g., a molecule having a plurality of phosphates in a phosphate chain, such as 2, 3, 4, 5, 6, 7, 8, 10, or more phosphates). A nucleotide may generally include adenosine (A), cytosine (C), guanine (G), thymine (τ) and uracil (U), or variants thereof. A nucleotide may include any subunit that can be incorporated into a growing nucleic acid strand. Such subunit can be an A, C, G, T, or U, or any other subunit that is specific to one or more complementary A, C, G, T, or U, or complementary to a purine (e.g., A or G, or variant thereof) or a pyrimidine (e.g., C, T, or U, or variant thereof). A subunit can enable individual nucleic acid bases of group of bases (e.g., AA, TA, AT, GC, CG, CT, TC, GT, TC, AC, CA, or uracil counterparts thereof) to be resolved. A nucleotide may be labeled or unlabeled. A labeled nucleotide may yield a detectable signal, such as an optical, electrostatic, or electrochemical signal.

The terms “polynucleotide,” “oligonucleotide,” “nucleotide,” “nucleic acid,” and “nucleic acid molecule” generally refer to a polymeric form of nucleotides (polynucleotides) of various lengths, either ribonucleotide (RNA) or deoxyribonucleotides (DNA). Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 20, 40, 100, 200, 300, 400, 500, 600, 00, 800, 21000, 1200, 1500, 2000, 5000, 12000, or more than 10000 nucleotides in length.

The terms “peptide,” “polypeptide,” and “protein” as used herein generally refer to a compound comprising amino acid residues covalently linked by peptide bonds. Polypeptides may include any peptide or protein comprising two or more amino acids joined to each other by peptide bonds. Examples of polypeptides may include, for example, biologically active fragments, substantially homologous polypeptides, oligopeptides, homodimers, heterodimers, polypeptides and variants thereof, modified polypeptides, derivatives, analogs, fusion proteins, or combinations thereof. A polypeptide may be a natural peptide, a recombinant peptide, or a combination thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The challenge of implementing a real-time detection method is creating a detection/analysis system that can specifically measure the concentration of analytes in a sample and/or quantify the specific interaction of the analytes with other biological and chemical structures (e.g., probes). Some real-time methods, such as infrared (IR) and ultraviolet (UV) spectroscopy techniques rely on absorption spectrum and signatures of biomolecules [see, references 1-3], whereas others like electroanalysis rely on interaction of biomolecules with oxidation-reduction systems biofunctionalized electrodes [see, references 4-5].

In this disclosure, specific optical real-time detection methods and the systems using such methods are described. These methods/systems may rely on time-resolved fluorescence systems and the corresponding biomolecular constructs involving time-resolved optical reporters. In some embodiments, the real-time methods may use affinity-based biosensor formats that include one or more capturing/detection probes that are immobilized on a solid surface, hereinafter referred to as a solid-phase capturing probe.

### Real-Time Detection System

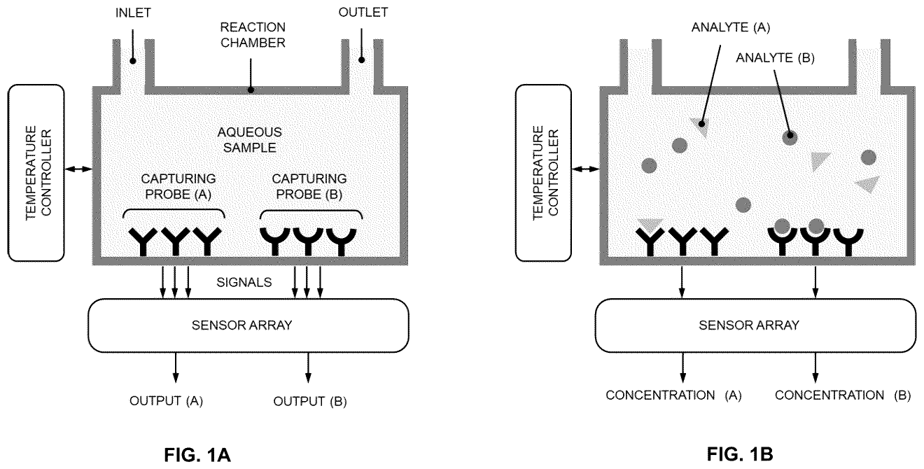

In FIGS. 1A-1B, the building blocks of a real-time detection systems are shown. As shown in FIG. 1A, the sample may be physically contained in a reaction chamber, and the insertion/introduction and removal of the sample may be carried out through a fluidic inlet and a fluidic outlet, respectively. The volume of the sample (within the reaction chamber) can be from about 1 nL to about 100 mL, and specifically from about 100 nL to about 100 μL. During the analysis, the temperature of the reaction chamber may be continually controlled by a temperature controller which may comprise one or more temperature sensors and heater and/or cooling elements. The controlled temperature range can be from about −20° C. to about 150° C. and specifically from about 25° C. to about 100° C. The heating and cooling rates can be as high as +50° C./sec, but in typical applications, as high as +15° C./sec. Within this reaction chamber, there may also exist one or more capturing (detection) probes immobilized on a surface/solid-phase of the reaction chamber. These capturing probes can be in an addressable format on the surface such that each capturing probe may interact with and/or capture one or more specific analytes at the addressable location. Many different capturing probe structures can be used in the present disclosure. Examples include, but are not limited to, nucleic acid sequences, aptamers, and antibodies, etc. The real-time detection system may also include a sensor array that continually monitors the capturing probe array, reports the interactions and/or capturing of the analytes at each addressable location, or produces electronic outputs of the received optical signals. As illustrated in FIG. 1B, the reported outputs of the sensor can be used to estimate the concentration of the analyte that is captured at the addressable location as a function of time. This time-resolved information/function can subsequently be correlated to the presence the analyte in the sample and/or the concentration of the analyte in the sample and/or the reactivity of the analyte in the sample with the corresponding capturing probe.

### Fluorescence-Based Analyte Detection

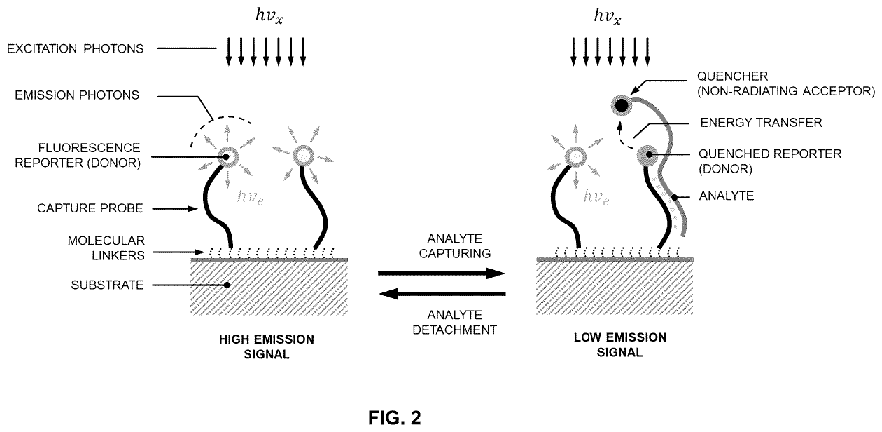

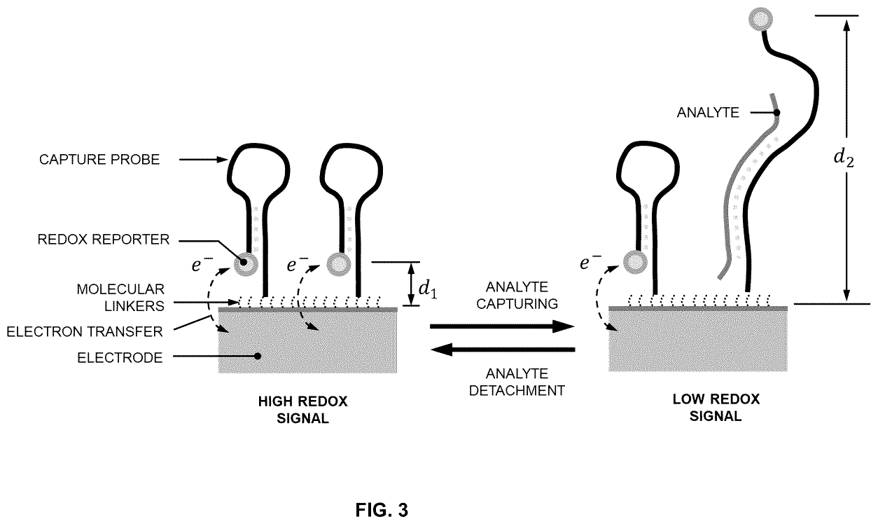

Different real-time detection systems can be designed. In FIG. 2 show two possible designs. For example, real-time continuous-wave fluorescence-based methods can use fluorescence-labeled capturing probes and quencher-labeled analytes to realize real-time sensing [3,6-8]. As illustrated in FIG. 2, capturing of analytes by the probe can bring the quencher (acceptor) into intimate proximity of the fluorescence reporter (donor), thereby terminating its photon emission through energy transfer. This reduction of signal can then be detected by a fluorescence detector and be used as a quantitative indication of the analyte capturing. In FIG. 3, there can be another real-time detection method that uses redox reporters in the capturing probes that are immobilized on an electrode system [9-11]. The capturing probes are specifically designed as a molecule switch such that analyte capturing can alter the state of the probe in terms of redox activity. As illustrated in FIG. 3, analyte capturing can change the state of the probe, push the redox reporter away from the surface, and prohibit electron transfer from that reporter. The reduction in electron transfer can then be detected by a cyclic voltammetry system or device and used as a quantitative indication of the analyte capturing.

### Time-Resolved Optical Reporter

Time-resolved optical reporter can be a molecular structure that can absorb photons with specific wavelengths (energies) and transition its electronic structure to an excited state and then subsequently return to the ground state while emitting photons with a higher wavelength (lower energy). The time delay between the photon absorption and emission may be probabilistic and can be described by an exponential random variable with lifetime t. In theory, all fluorophore molecules including organic, metallo-organic and quantum dots, can be used as time-resolved optical reporters. However, fluorophores with lifetimes greater than 10 ns are preferred. Examples are ruthenium, chromium and lanthanide chelate constructs [12-13]. Some lanthanide chelate complexes can emit strong fluorescence with distinct physical properties that are different from those of organic fluorescent compounds. For example, the fluorescence of lanthanide complexes can be long-lived with the half decay-time (lifetime) of several hundred microseconds to 2 milliseconds.

### Time-Resolved Optical Detection

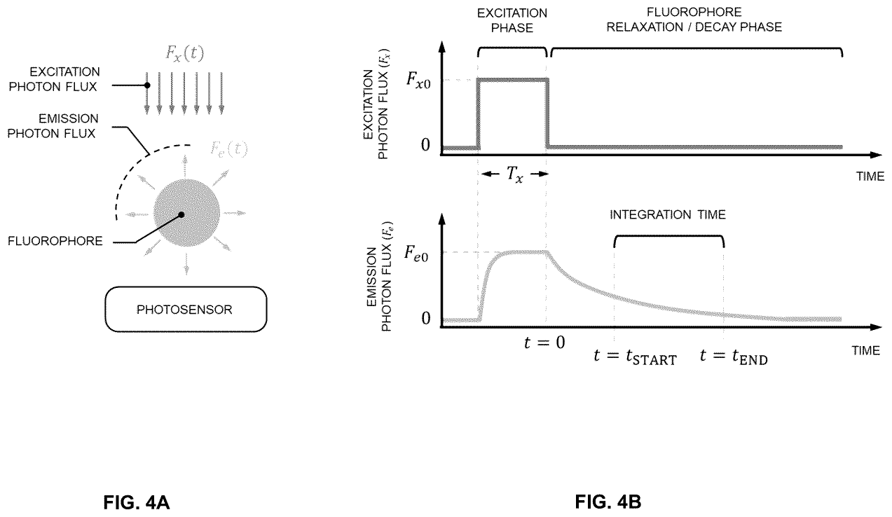

Time-resolved optical detection, broadly defined, can be the detection of the fluorescence emission from a molecular construct (such as the time-resolved optical reporter) only after the incident excitation photon flux is terminated. In time-resolved optical detection, the detection is not concurrent with the excitation, i.e., the excitation flux is absent during the detection. This may be the main difference between time-resolved optical detection method and continuous-wave fluorescence-based detection method in which excitation is continuous. As a result, the detection system and analysis approach in time-resolved optical detection methods are different compared to the continuous fluorescence-based, which is widely known in the art. In FIGS. 4A-4B, the concept as well as the basic excitation and emission waveforms of time-resolved optical detection are illustrated. The excitation photon flux is denoted by Fₓ (t) and is typically applied as a pulse or series of short pulses during the excitation phase with duration of Tₓ. The fluorescence emission from the reporter (or fluorophore), Fₑ (t), is a function of Fₓ (t) but has a finite lifetime of t. This means that once Fₓ (t) is terminated (or during the relaxation/decay phase), Fₑ (t) continues to emit photons, but the emission amplitude decays with the lifetime (half-life) of τ, i.e., Fₑ (t) ∝e^{-t/t}. Therefore, one can collect and measure the emitted photons during a time period in the relaxation/decay phase to evaluate the status and/or quantity of optical (fluorescence reporters) in the system.

If one assumes that the excitation photon flux Fₓ (t) has the amplitude of Fₓ₀ and is terminated at t=0, then Fₑ (t) at t≥0 can be formulated by equation (1):

$${F}_{e} ( t ) = {F}_{e 0} {e}^{- \frac{t}{τ}} = α [ N ] {F}_{x 0} {e}^{- \frac{t}{τ}} ; \qquad ( 1 )$$

where Fₑ₀ is the steady-state emission during the excitation phase, α is the excitation-to-emission yield and [N] is the concentration of the fluorescence reporters. Now if the photon collection is carried out between t_{START} and t_{END} during the relaxation/decay phase, then the total collected photons, Yₚₕ, becomes equation (2):

$${Y}_{ph} = {\int }_{{t}_{START}}^{{t}_{END}} {F}_{e 0} {e}^{- \frac{t}{τ}} dt = {τ F}_{e 0} ( {e}^{- {t}_{START} / τ} - {e}^{- {t}_{END} / τ} ) . \qquad ( 2 )$$

It is important to point out that equation (2) contains information including of not only the fluorescence reporter concentration ([N]), but also the fluorophore lifetime, t which can be used as another independent analysis parameter originating from the fluorophore. This is imperative and a notable advantage over continuous-wave fluorescence methods. In addition, time-resolved methods, once implemented, do not require complex optical filtering and wavelength selective imaging to block the excitation photon flux as the measurement and analysis are performed at time instances where no excitation flux is present. It is known in the art that implementation of wavelength selective filters can be complex and expensive which makes no filter requirement of time-resolved optical methods as another advantage over continuous-wave fluorescence methods.

### Design of Probe and Analyte Moieties

This present disclosure provides surface-immobilized detection (capturing) probe that can interact with the target moieties such that unique time-resolved optical signals can created with time and amplitude signatures that are indicative of the analytes interacting with and/or being captured by the probe. These constructs can all be used to create a real-time biosensing system that allows measuring the emission photon flux after the excitation photon flux is terminated.

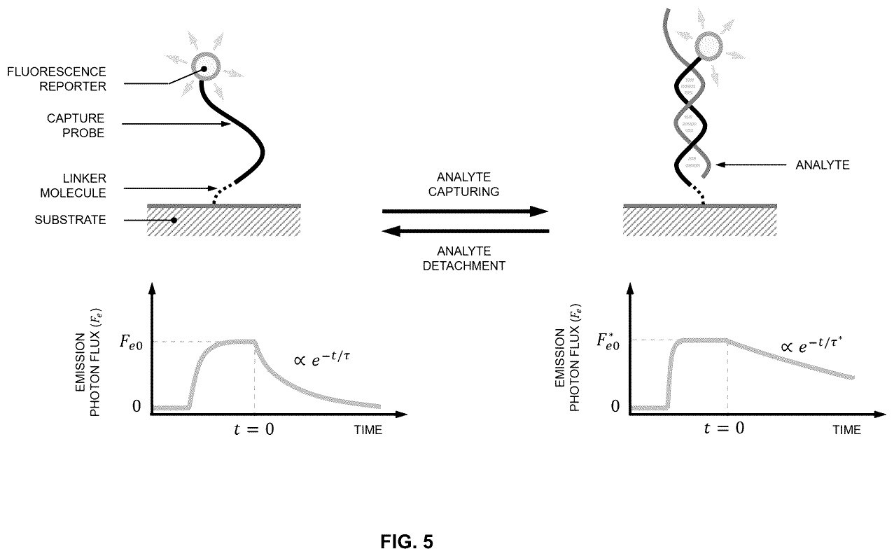

In FIG. 5, a first method of creating a time-resolved optical signal is shown. A capturing probe can comprise a fluorescence reporter and can be immobilized on a solid substrate. The analyte does not bear a reporter molecule. In this system, the fluorescence signal comes from the reporter on the capturing probe. In some embodiment, the capturing probe is immobilized via a linker molecule attached to the surface of the solid substate. This probe, once excited with a photon flux pulse with wavelengths appropriate for its fluorescence reporter, can emit a time-resolved signal with lifetime of t. Now, when the analyte is captured, the capturing probe structure becomes more rigid and confined. Therefore, the lifetime of the reporter increases to τ*, where τ*>τ. This typically also increases the steady-state emission amplitude from Fₑ₀ to

$${F}_{e 0}^{*} ,$$

as depicted on FIG. 5.

It is important to explain the underlying principle of this phenomenon. The lifetime of fluorescence reporters depends on the characteristics and dynamics of the relaxation paths that are available for its electronic excited state to return to the ground state. One of the relaxation paths is radiating relaxation which emits a photon. By subjecting fluorescence reporters to either intermolecular, or intramolecular collisions, one can increase the probability of non-radiating relaxations and consequently reduce the observed fluorescence lifetime. The opposite scenario is also valid. If one can remove collisions and essentially freeze or confine the fluorescence reporter, one can in theory increase the observed lifetime of the emission and also increase the total photon emission rate [14]. Making the fluorophore reporter confined and placed in a more rigid structure that is subject to less molecular collisions can be one way to mitigate the lifetime reduction.

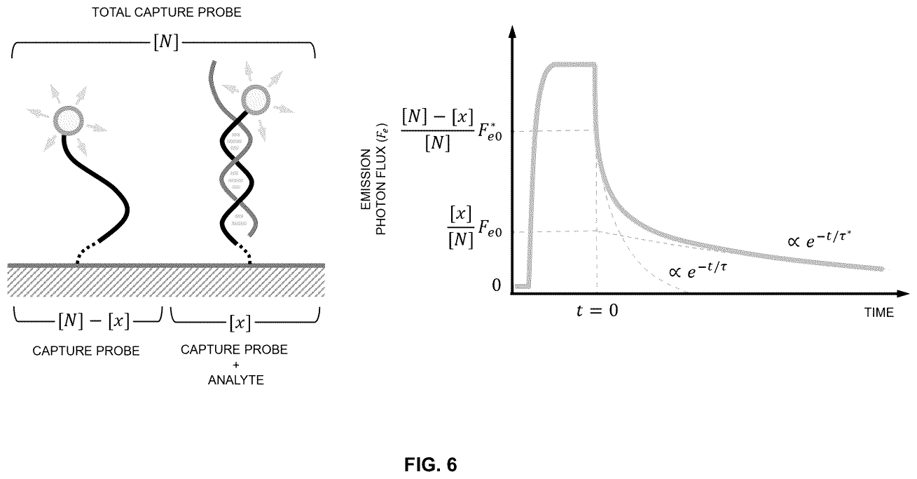

FIG. 6 shows the photon emission waveform of a biosensor that uses the method of FIG. 5. The total concentration of the probes at the surface is [N] and the captured analyte concentration is [x], making the concentration of free probes [N]-[x]. As shown, the emission waveform can consist of two exponentially decaying signals. As the surface captures more analytes, the observed decay becomes more dominated by the exponential decay with the lifetime of τ*. Therefore, by measuring the decay and computing the amplitudes of both exponential decays, one can estimate the ratio of [N] and [x] at that capturing spot. For example, the decay profiles of the capturing probes alone (in the absence of the analyte) and analyte-capturing probe duplex alone (in the presence of excess analytes to ensure substantially all capturing probe bound with the corresponding analytes) can be observed and measured to establish the boundaries for lifetimes for this construct.

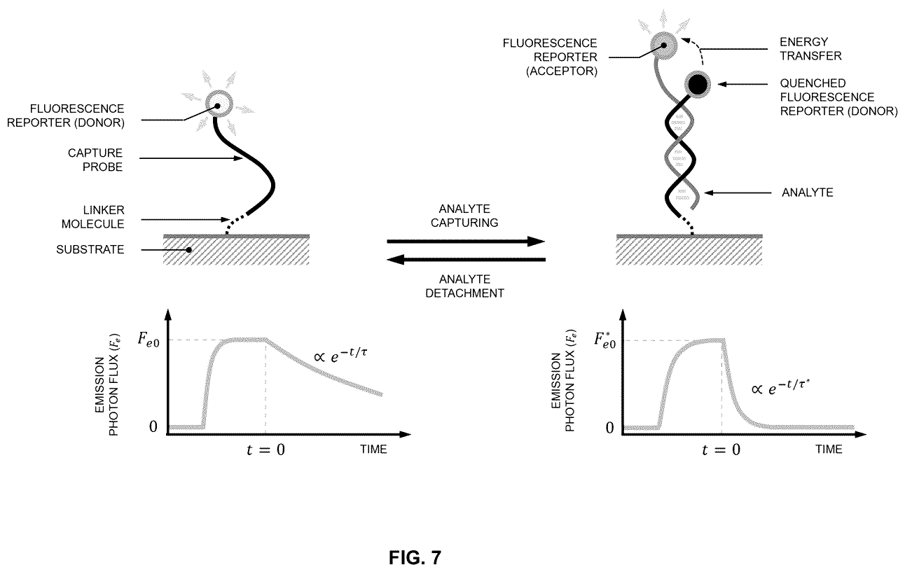

In FIG. 7, a second method of creating a time-resolved optical signal is shown. A capturing probe can comprise a first fluorescence reporter and can be immobilized on a solid substrate. This probe, once excited with a photon flux pulse with wavelengths appropriate for its fluorescence reporter, emits a time-resolved signal with lifetime of T. In this system, the analyte is labeled with a second fluorescence reporter that has a lifetime of τ*. The first fluorescence reporter for the probe, the second fluorescence reporter for the analyte, and their fluorescence emission and absorption spectra are selected in such a way that the first and second fluorescence reporters can form a fluorescence resonance energy transfer (FRET) system when placed in intimate proximity (e.g., <5 nm) in which the probe fluorescence reporter is the donor while the analyte fluorescence reporter is the acceptor. Based on this configuration, when the analyte capturing occurs, the donor and acceptor are brought together to form a FRET system, and the radiating relaxation predominantly occurs through the analyte reporter. Therefore, the lifetime of the emission changes to τ* from τ and the amplitude changes to

$${F}_{e 0}^{*}$$

from Fₑ₀ after the analyte is captured by the capturing probe.

It is important to mention that there are many different methods in the art to label the analytes, i.e., attach reporter molecules to analytes permanently. For example, as provided in the present disclosure, the analytes can be labeled by using primers attached with reporter molecule (e.g., a label) in a PCR reaction to provide amplified products. The labeled primers may be configured to facilitate and/or enable the detection and/or monitoring of the presence, quantity, concentration or binding activity of the primers, amplicons, or other analytes. The reporter molecule may be incorporated into the amplified products as the amplification reaction proceeds. The primers may be labeled with one or more reporter molecules. The reporter molecules can be optical. Examples of reporter molecules may include, but are not limited to fluorescent, quenchers, fluorophores, members of a fluorescence resonance energy transfer (FRET) pair, redox species, or combinations thereof. Another way to introduce the labels is to conduct post synthesis chemical reaction on the primer. For example, reactive chemical functional groups (e.g., leaving groups such as halides, alkyne, alkene, additional primary amine/hydroxy group) can be introduced to the analyte when the primer or other shorter nucleic acid sequence are attached to the analyte such that the analyte incorporates these reactive chemical functional groups. Then a label comprising another reactive chemical functional group reacts with the incorporated reactive chemical functional group on the analyte, thereby attaching the label to the analyte via a chemical bond (e.g., an amide bond, an ester bond, a carbon-carbon bond, a —NH— or —O— bridge, etc.).

FRET pairs can include but are not limited to 6-FAM (donor) and LC Red 640 or Alexa Fluor 546 (acceptors); fluorescein (donor) and tetramethylrhodamine (TMR, acceptor); IAEDANS (donor) and fluorescein (acceptor); EDANS (donor) and Dabcyl (acceptor); fluorescein (donor) and fluorescein (acceptor); BODIPY FL (donor) and BODIPY FL (acceptor); and fluorescein (donor) and QSY 7 and QSY 9 dyes (acceptors).

The donors of FRET pairs can include but are not limited to lanthanide chelates and ruthenium chelates. Examples of lanthanide chelates are shown below:

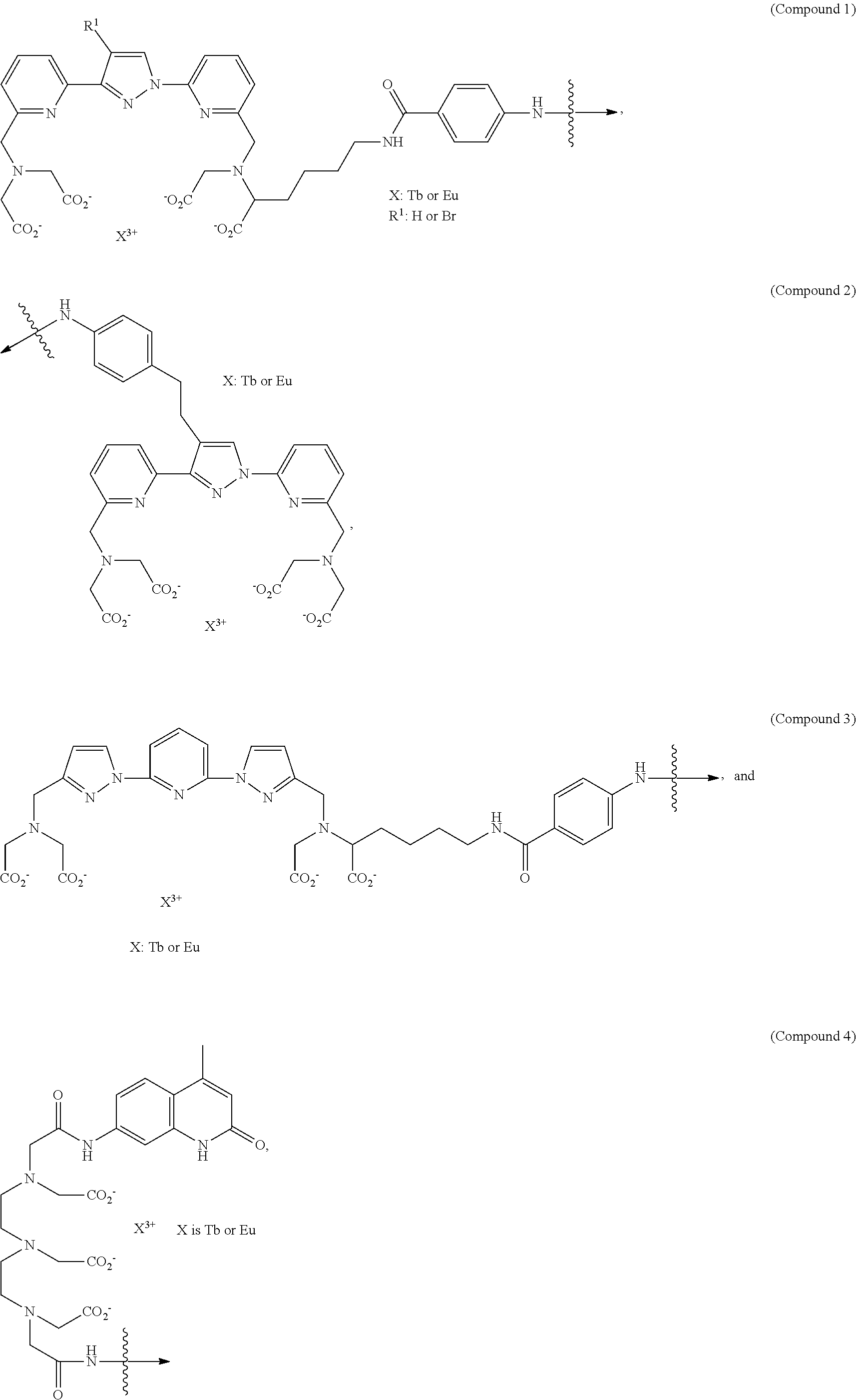

which can form a FRET pair with tetramethylrhodamine (TMR, acceptor), wherein the complexed metal ion X³⁺ in Compounds 1-4 can be terbium (III) or europium (III) or other lanthanide ions (La³⁺, Ce³⁺, Pr³⁺, Nd³⁺, Pm³⁺, Sm³⁺, Gd³⁺, Dy³⁺, Ho³⁺, Er³⁺, Tm³⁺, Yb³⁺, or Lu³⁺), and wherein indicates a connection

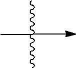

indicates a connection with a nucleic acid molecule or a linker to a nucleic acid molecule. See P. R. Selvin et al., (1994) J. Am. Chem. Soc. 116:6029-6030; P. R. Selvin and J. E. Hearst (1994) Proc. Natl. Acad. Sci. USA 91:10024-10028; and U.S. Pat. No. 5,859,215, each of which is incorporated in its entirety. Examples of the ruthenium chelates are shown below:

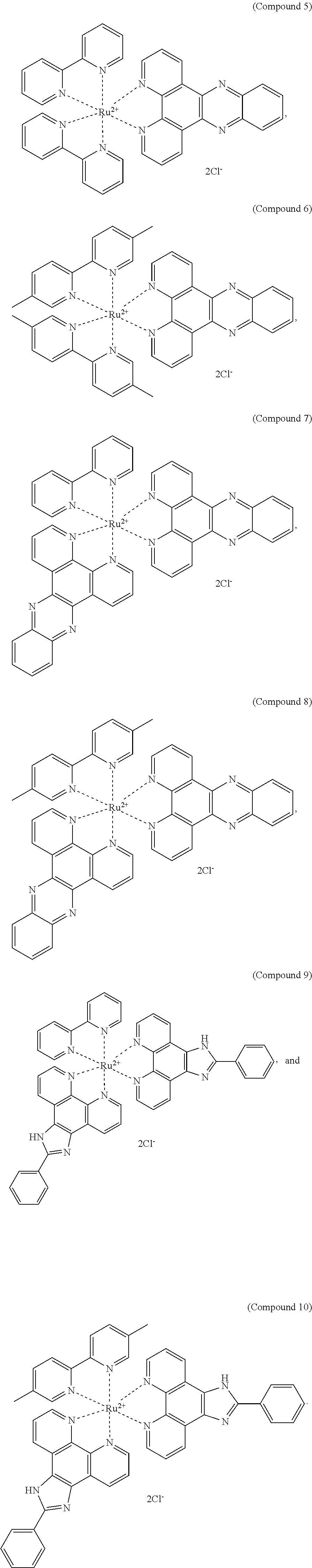

These ruthenium chelates can be used as donor when paired with Cy5,5 dye as the radiating acceptor. See C. E. Elgar et al. (2023) J. Am. Chem. Soc. (2023) 145 (2): 1236-1246, which is incorporated by reference in its entirety.

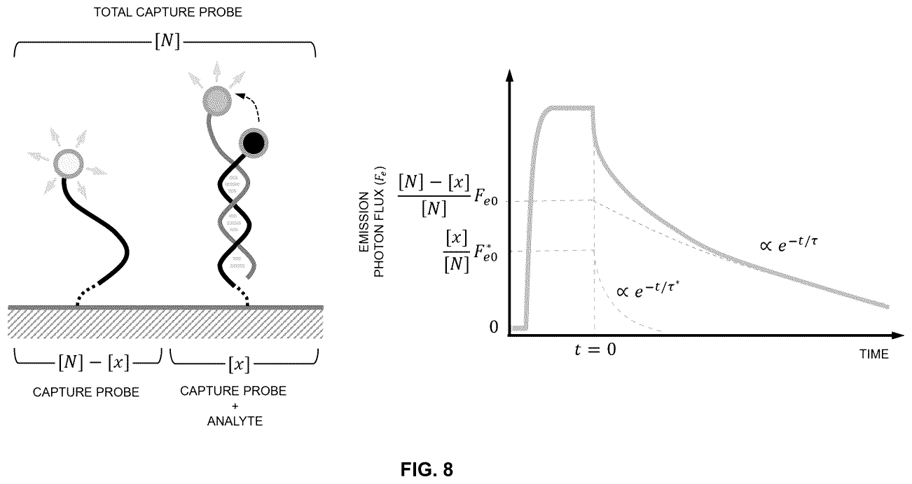

FIG. 8 shows the photon emission waveform of a biosensor that uses the method of FIG. 7. The total concentration of the probes at the surface is [N] and the captured analyte concentration is [x], making the concentration of free probes as [N]-[x]. As shown, the emission waveform can consist of two exponential decay signals. As the surface captures more analytes, the observed decay may become more dominated by the exponential decay with lifetime of τ*. Therefore, by measuring the decay and computing the amplitudes of both exponential decays, one can estimate the ratio of [N] and [x]. For example, the decay profiles of the capturing probes alone (in the absence of the analyte) and analyte-capturing probe duplex alone (in the presence of excess analytes to ensure substantially all capturing probe bound with the corresponding analytes) can be observed and measured to establish the boundaries of lifetimes for this construct.

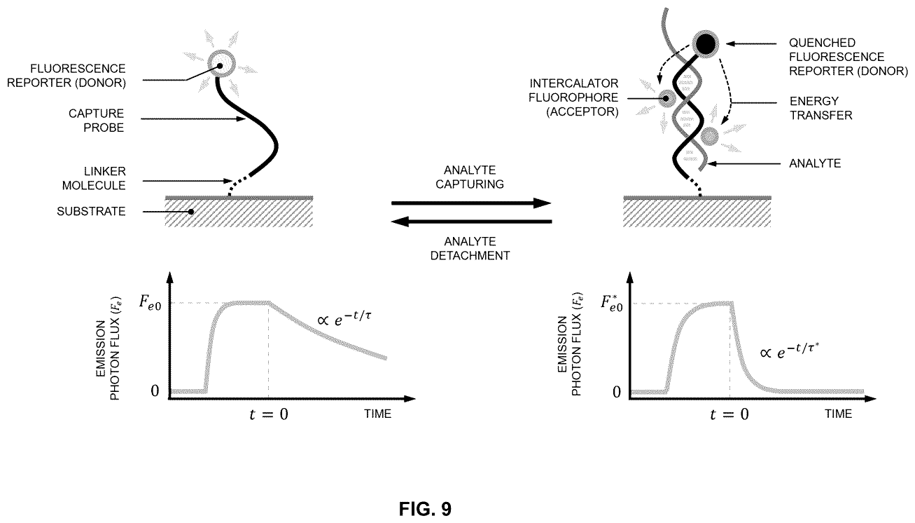

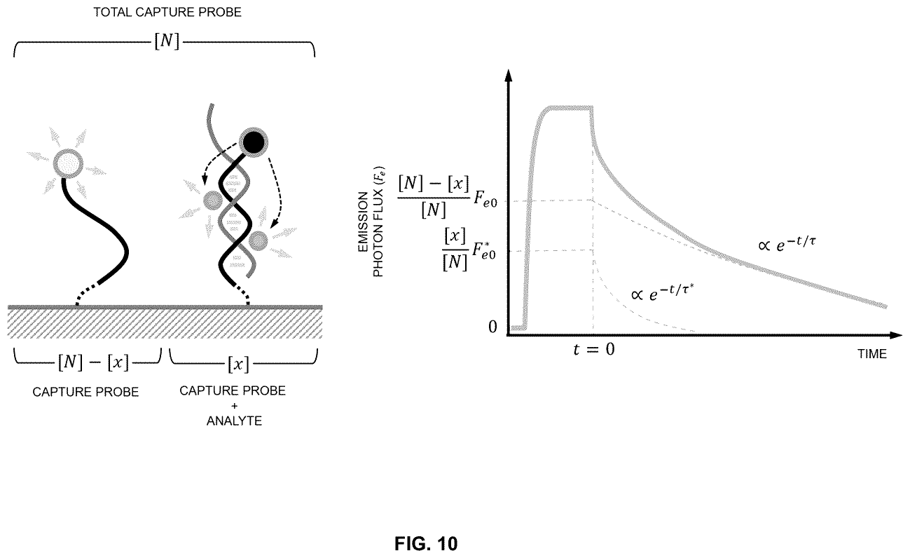

In FIG. 9, a third method of creating a time-resolved optical signal is shown. A capturing probe that comprises a first fluorescence reporter can be immobilized on a solid substrate. This probe, once excited with a photon flux pulse with wavelengths appropriate for its fluorescence reporter, may emit a time-resolved signal with lifetime of t. In this system, the analyte is not labeled. However, once the analyte is captured by the probe, the captured structure (duplex) may facilitate the attachment of one or more free roaming second fluorescence reporter molecules, hereinafter referred to as the intercalator reporter, that has a lifetime of τ*. The spectral characteristics of the fluorescence reporter of the probe and the intercalator reporter may be selected such that they can form a fluorescence resonance energy transfer (FRET) system when placed in intimate proximity (e.g., <5 nm) in which the probe reporter is the donor while the intercalator reporter is the acceptor. Based on this, when the analyte capturing event occurs, the donor and acceptor are brought together to form a FRET system, and the radiating relaxation may predominantly occur through the intercalator reporter. Therefore, the lifetime of the relaxation (decay) switches to τ* from t, and the amplitude switches F*ₑ₀ from Fₑ₀.

In this example, different intercalator reporters can be used. Examples of intercalator reporters are those molecules that can bind to double-stranded DNA (dsDNA) such as SYBR® Green (available from Biotium (Hayward, CA)), EvaGreen (see F. Mao et al. BMC Biotechnol (2007) 7, 76, which is incorporated by reference by its entirety, available from Biotium (Hayward, CA)) or short modified DNA sequences in the form of hybridization or TaqMan probes, to name a few. Fluorescent intercalator reporters can be small molecules that fluoresce when intercalated in or bound to double stranded nucleic acids. In some embodiments, the level of fluorescence that is emitted by a fluorescent intercalator is significantly higher when the intercalator is intercalated in or bound to the double stranded nucleic acid, as compared to the basal level of fluorescence when unbound (or free in solution). In some embodiments, the fluorescent DNA intercalator can exhibit a very low level of fluorescence when unbound to double-stranded nucleic acid. For example, the intercalator reporters (fluorescent intercalator) can be 9-aminoacridine, ethidium bromide, a phenanthridine dye, green fluorescent nucleic acid dye EvaGreen®, 2-[bis [3-(dimethylamino) propyl]amino]-4-[(3-methyl-2 (3H)-benzothiazolylidene) methyl]-1-phenylquinolinium PicoGreen® (P-7581, Molecular Probes), propidium iodide (P-4170, Sigma), acridine orange (A-6014, Sigma), thiazole orange, oxazole yellow, 7-aminoactinomycin D (A-1310, Molecular Probes), cyanine dyes (e.g., TOTO®, YOYO®, BOBOR, and POPOR), SYTOR, SYBR® Green I (N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR® Green II, SYBR® DX, OliGreen®, CyQuant® GR, SYTOX® Green, SYTOR9, SYTOR10, SYTOR17, SYBR®14, FUN®-1, DEAD™ Red, hexidium iodide, dihydroethidium, ethidium homodimer, 9-amino-6-chloro-2-methoxyacridine, DAPI, DIPI, indole dye, imidazole dye, hydroxystilbamidine (FluoroGold™), LDS 751, and the dyes described in Georghiou, Photochemistry and Photobiology, 26:59-68, Pergamon Press (1977); Kubota, et al., Biophys. Chem., 6:279-284 (1977); Genest, et al., Nucleic Acids Res., 13:2603-2615 (1985); Asseline, EMBO 1, 3:795-800 (1984); Richardson, et. al., U.S. Pat. No. 4,257,774; and Letsinger et. al., U.S. Pat. No. 4,547,569. In some embodiments, the fluorescent DNA intercalator is SYBR® Green I, EvaGreen®, PicoGreen®, ethidium bromide, unsymmetrical cyanine dye 2-[N-(3-dimethylaminopropyl)-N-propylamino]-4-[2,3-dihydro-3-methyl-(benzo-1,3-thiazol-2-yl)-methylidene]-1-phenyl-quinolinium SYBR® Gold, 1′-(4,4,7,7-Tetramethyl-4,7-diazaundecamethylene)-bis-4-(3-methyl-2,3-dihydro-(benzo-1,3-oxazole)-2-methylidene)-quinolinium tetraiodide (YOYO®), 4-[(3-methyl-1,3-benzoxazol-2 (3H)-ylidene) methyl]-1-[3-(trimethylammonio) propyl]quinolinium diiodide YO-PRO™, 1′-(4,4,7,7-tetramethyl-4,7-diazaundecamethylene)bis-4-(3-methyl-2,3-dihydro (benzo-1,3-thiazole)-2-methylidene) quinolinium TOTO®, 4-[6-(Benzoxazol-2-yl)-dihydro-3-methyl-2 (3H)-benzothiazolylidenemethyl]-1-methylquinolinium iodide (BOXTO™), or 4-[(3-methyl-6-(benzothiazol-2-yl)-2,3-dihydro-(benzo-1,3-thiazole)-2-methylidene)]-1-methyl-pyridinium iodide (BEBO™). In some embodiments, the intercalator reporters is SYBR® Green I.

FIG. 10 shows the photon emission waveform of a biosensor that uses the method of FIG. 9. The total concentration of the probes at the surface is [N] and the captured analyte concentration is [x], making the concentration of free probes [N]-[x]. As shown, the emission waveform may consist of two exponentially decaying signals. As the surface captures more analytes, the observed decay may become more dominated by the exponential decay with lifetime of τ*. Therefore, by measuring the decay and computing both the amplitude of both exponential decays, one can estimate the ratio of [N] and [x] in the capturing spot. For example, the decay profiles of the capturing probes alone (in the absence of the analyte) and analyte-capturing probe duplex alone (in the presence of excess analytes to ensure substantially all capturing probe bound with the corresponding analytes) can be observed and measured to establish the boundaries of lifetimes for this construct. An additional experiment may be to record the emission profiles of the free intercalators in the solution in the absence of the probe and the analyte to establish the background interference, if there is any.

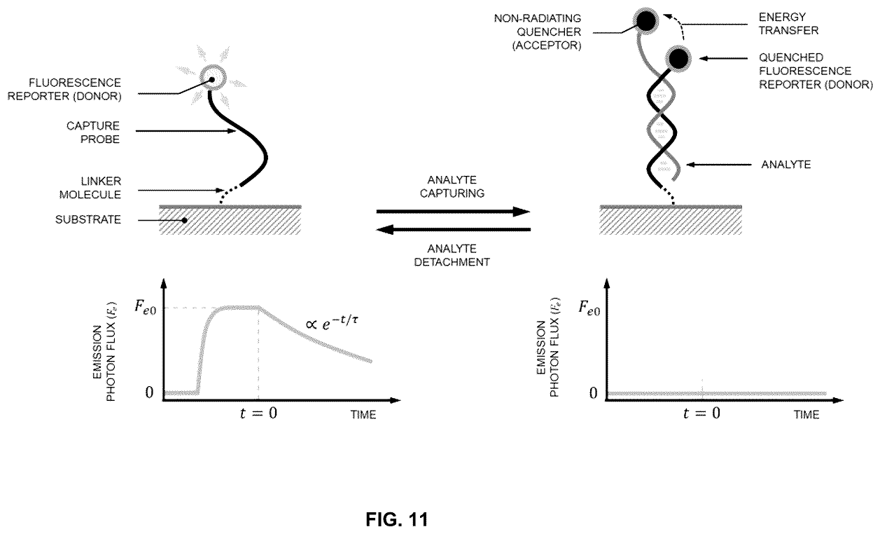

In FIG. 11, the fourth method of creating a time-resolved optical signal is shown. A capturing probe that includes a fluorescence reporter can be immobilized on a solid substrate. This probe, once excited with a photon flux pulse with wavelengths appropriate for its fluorescence reporter, may emit a time-resolved signal with lifetime of t. In this system, the analyte may be labeled with a non-radiating quencher that can act as the energy acceptor for the fluorescence reporter that is on the probe, once the non-radiating quencher is placed in the intimate proximity (e.g., <3 nm) of the fluorescence reporter. Based on this configuration, when the analyte capturing occurs, the donor and acceptor can be brought together to quench the optical signal from the fluorescence reporter completely.

A non-radiating optical quencher or quencher or non-radiating quencher can be a compound (e.g., a small molecule dye) that quenches a signal emitted from a fluorescent compound, e.g., a fluorescent intercalator or a fluorescence reporter. In some embodiments, the quencher absorbs excitation energy from the fluorescent compound (e.g., fluorescent intercalator or fluorescent reporter) and dissipates the energy that is absorbed from the fluorescent compound as heat (not a new fluorescence signal). Typically, quenchers have an absorption spectrum that overlaps with the emission spectrum of the donor.

In some embodiments, the quencher is a small molecule dye that absorbs excitation energy from a fluorophore. Quencher can be referred to as a “dark quencher”, indicating the non-radiating characteristics. Example quenchers include, but are not limited to, Black Hole Quencher® (BHQ®) dyes (e.g., BHQ®-0, BHQ®-1, BHQ®-2, and BHQ®-3; LGC Biosearch Technologies, Petaluma, Calif.), Iowa Black® Dark Quenchers (e.g., Iowa Black® FQ and Iowa Black® RQ; Integrated DNA Technologies, Inc., Skokie, Ill.), DABCYL™ QXL™ Quenchers (AnaSpec, Inc. Fremont, Calif.), IRDye® QC-1 (LI-COR Biosciences, Lincoln, Nebr.), and the quenchers described in WO 2001/086001 and US 2005/0164225, each of which is incorporated by reference in its entirety.

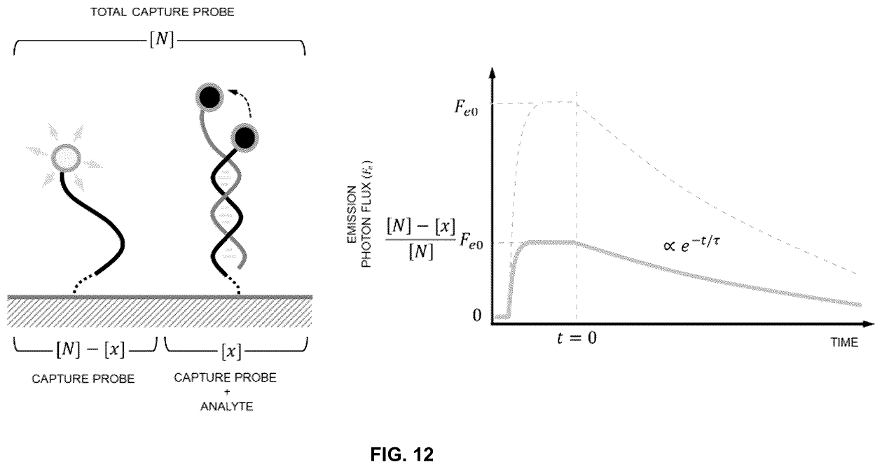

FIG. 12, shows the photon emission waveform of a biosensor that uses the method of FIG. 11. The total concentration of the probes at the surface is [N] and the captured analyte concentration is [x], making the concentration of free probes [N]-[x]. As shown, the emission waveform may consist of one exponentially decaying signal originating from the fluorescent reporter. As the surface captures more analytes, the observed amplitude may reduce while the lifetime remains the same. Therefore, by measuring the amplitude of decaying signal with and without the analyte present, one can estimate both [N] and [x] in that capturing spot. For example, the decay profiles of the capturing probes alone (in the absence of the analyte) and analyte-capturing probe duplex alone (in the presence of excess analytes to ensure substantially all capturing probe bound with the corresponding analytes) can be observed and measured to establish the boundaries of lifetimes for this construct.

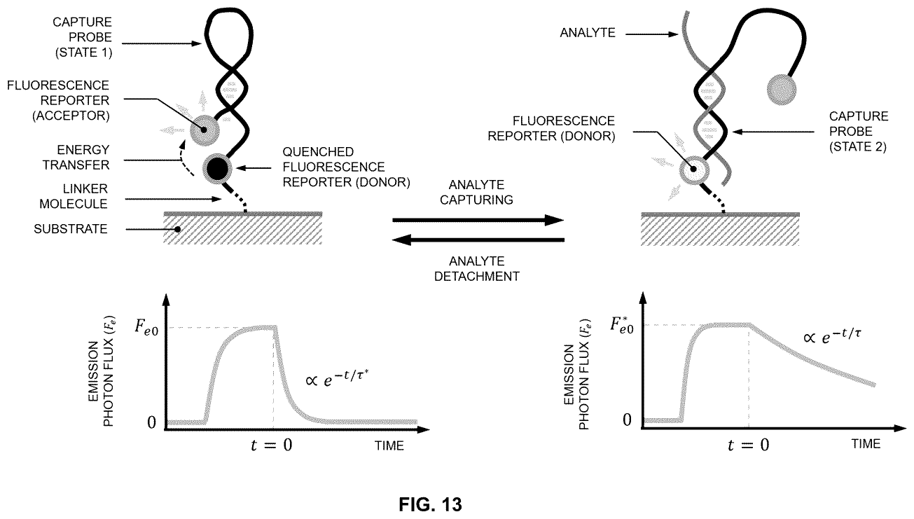

In FIG. 13, a fifth method of creating a time-resolved optical signal is shown. In this method, the capturing probe that is immobilized on the surface can include a FRET system with two fluorescence reporters in which the donor has a lifetime of t while energy acceptor has a lifetime of τ*. This probe, once excited with a photon flux pulse with wavelengths appropriate for its donor reporter, can emit a time-resolved signal either with lifetime of τ, or τ* and amplitude of Fₑ₀ and

$${F}_{e 0}^{*} ,$$

respectively which depends on the distance between its donor and acceptor. Now, the structure of the probe may be designed such that in the absence of the analyte (i.e., uncaptured state, hereinafter referred to as State 1), the State 1 configuration of the probe may establish a FRET system to emit photons whose lifetime is τ*. When the analyte is captured however (hereinafter referred to as State 2), the State 2 configuration of the probe may force the donor and acceptor move further from each other, and the FRET system is broken/negated, thereby the observed lifetime switches to T.

There are many ways to incorporate molecular switches on a probe that can change their observable states before and after capturing an analyte can be formed. Molecular switch is a structure that can help detect the state of a probe between a nucleic acid hybridization state or a non-hybridization state. The particular probe can comprise a “molecular switch” region for use as a hybridization probe. In some embodiments, this molecular switch region can be in an “open” (non-hybridized, mismatch) or “closed” (hybridized, match) position, while the probe as a whole remains in part hybridized to the target sequence. See U.S. Pat. No. 8,679,789, the content of which is incorporated by reference in its entirety. In the switch open position, the molecular switch can self-hybridized (forming a hairpin conformation) to bring the two fluorescence reporters on the probe within a proximity to form a FRET system or a donor/quencher system (depending on the properties of the receptor fluorescence reporter). This switch open position indicates a non-hybridization between the probe and the other nucleic acid. In the switch closed position, the molecular switch can hybridize with the target analyte, and indicate the presence of a complementary nucleic acid sequence between the probe and the target analyte.

In general, the molecular switch function can be provided by a switch domain in the probe. This switch domain may contain at least two features: (i) a binding domain that contains nucleic acid residues complementary to the target analyte, and (ii) a bridging domain that physically separates the binding domain from other parts of the probe that may bind to the target analyte. The binding domain may be at least 75% complementary in the case of a match to the target analyte. The bridging domain contains non-hybridizing universal, generic or mismatched bases, providing enhanced sensitivity towards the target analyte. The switch domain may be able to discriminate between (i) nucleic acid residues of a target analyte that are complementary to the binding domain (a “match”) on the probe, and (ii) nucleic acid residues of another nucleic acid that contains at least one nucleic acid residue that is not complementary to the binding domain (a “mismatch”); even under conditions wherein another part of the probe forms a stable duplex with part of the target analyte.

In some embodiments, the signal lifetime and/or amplitude detected from a fluorescence label (reporter) of the probe is determinative of the hybridization status of the switch domain. The “hybridization status”, as used herein, refers to whether the switch domain is open (mismatch, self-hybridized) or closed (match, forming a double-strand with target analyte). The amount of lifetime is determinative of the hybridization status when, under appropriate environmental conditions where the switch domain is duplexed with the target analyte, the label(s) can provide a mechanism to determine whether or not the switch domain is in proximity to either a matched or unmatched target analyte. In some embodiments, the amount of lifetime and/or amplitude detected from the fluorescence label is decreased when the switch domain is not hybridized to the target nucleic acid (mismatch). This decrease is relative to amount of lifetime and/or amplitude detected from the fluorescence label when the switch domain is hybridized to the target nucleic acid (match).

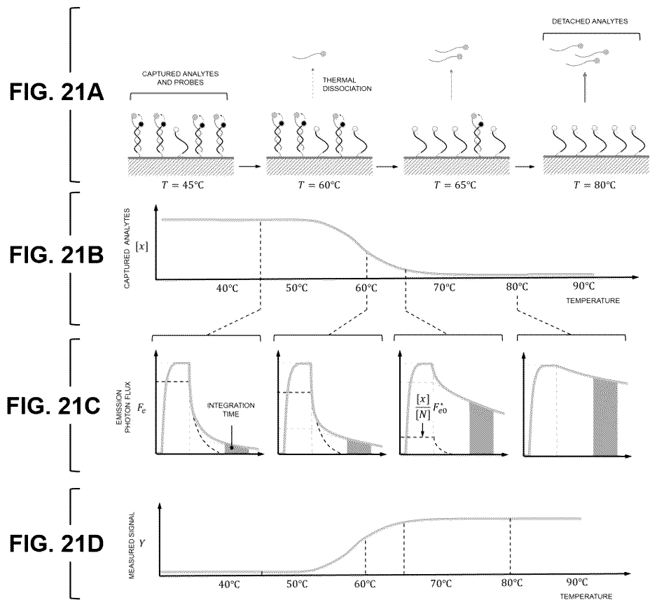

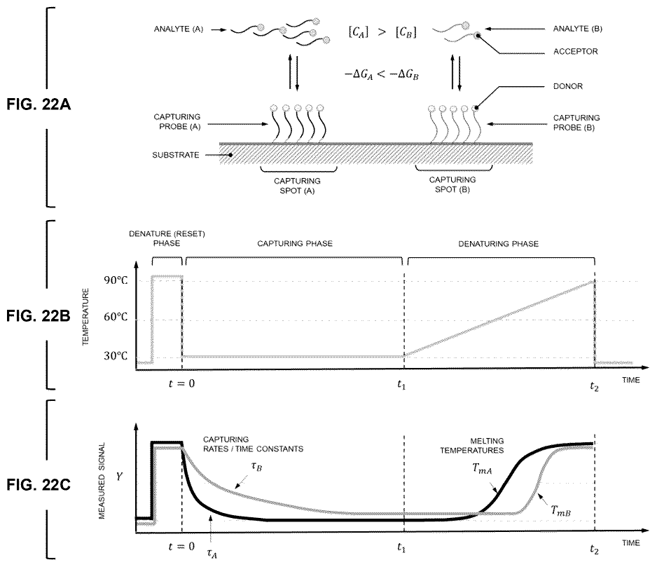

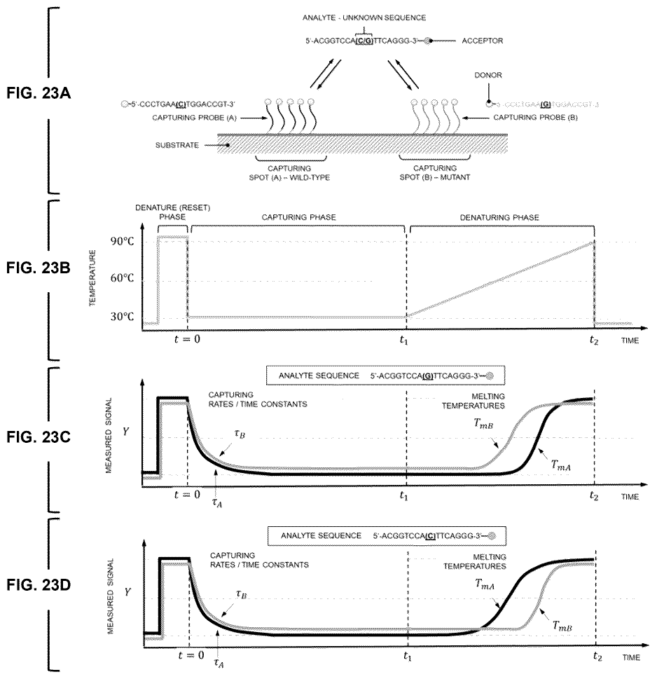

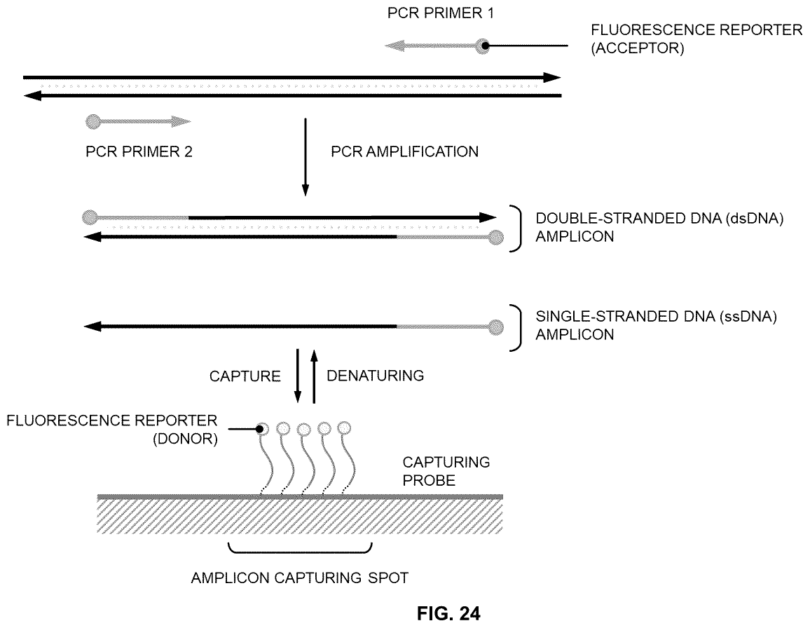

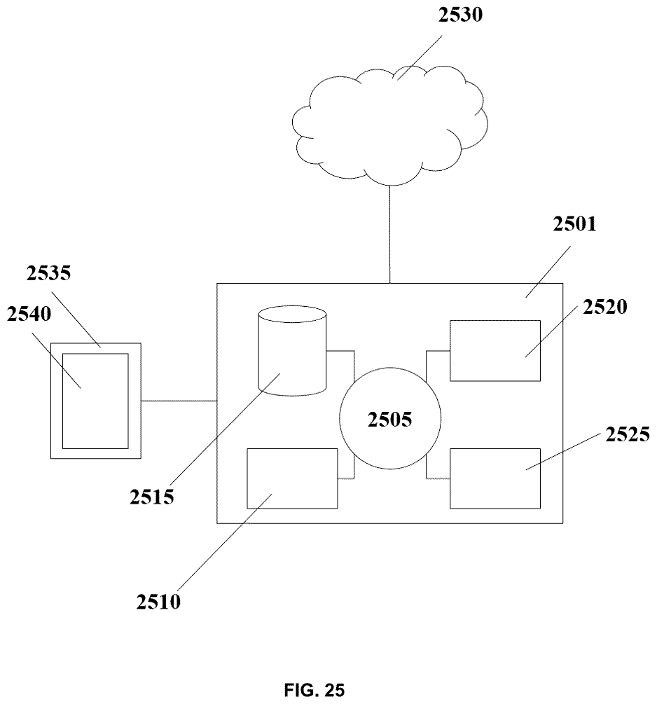

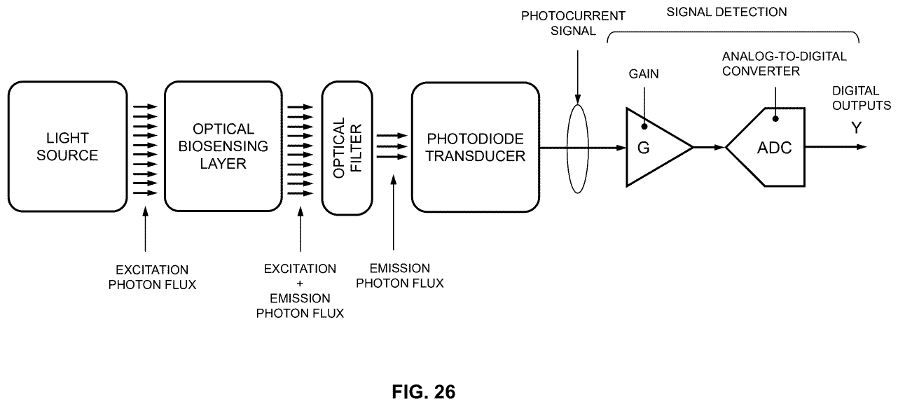

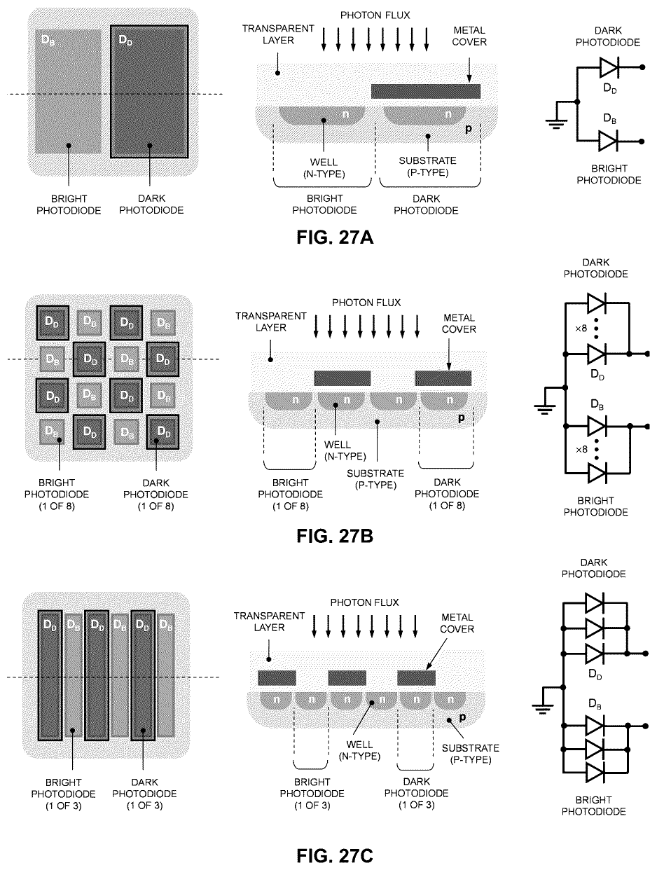

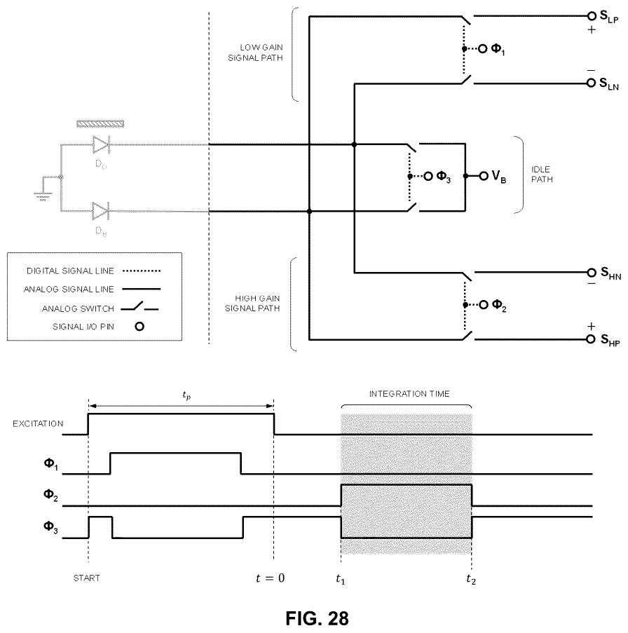

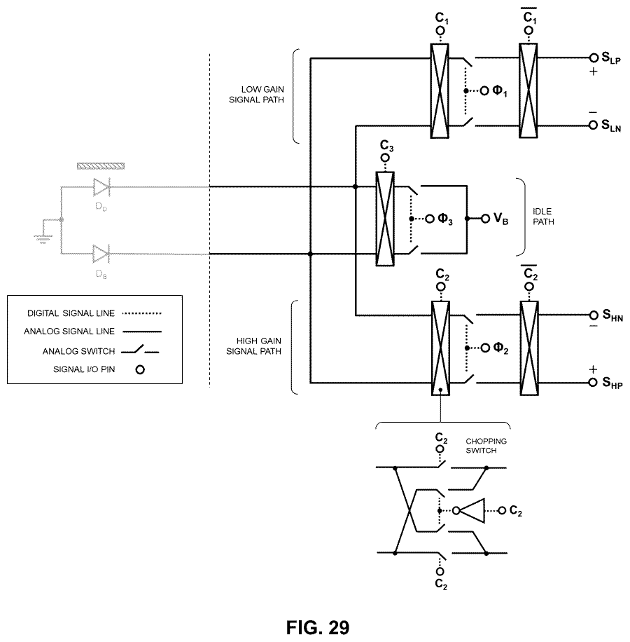

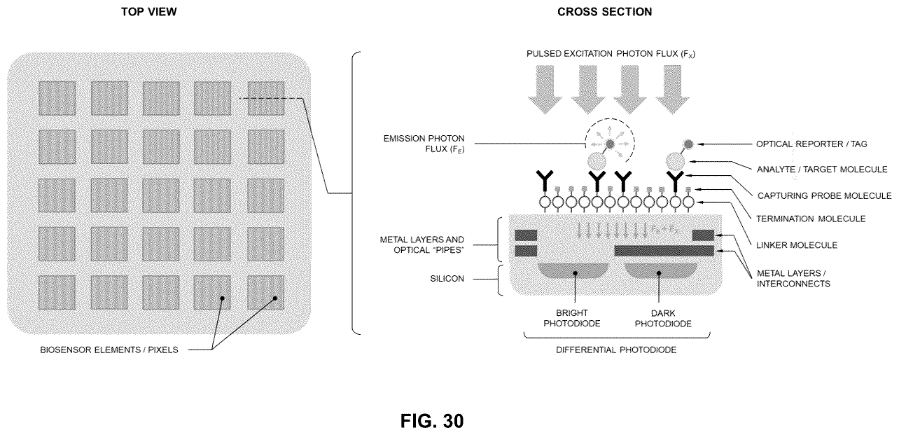

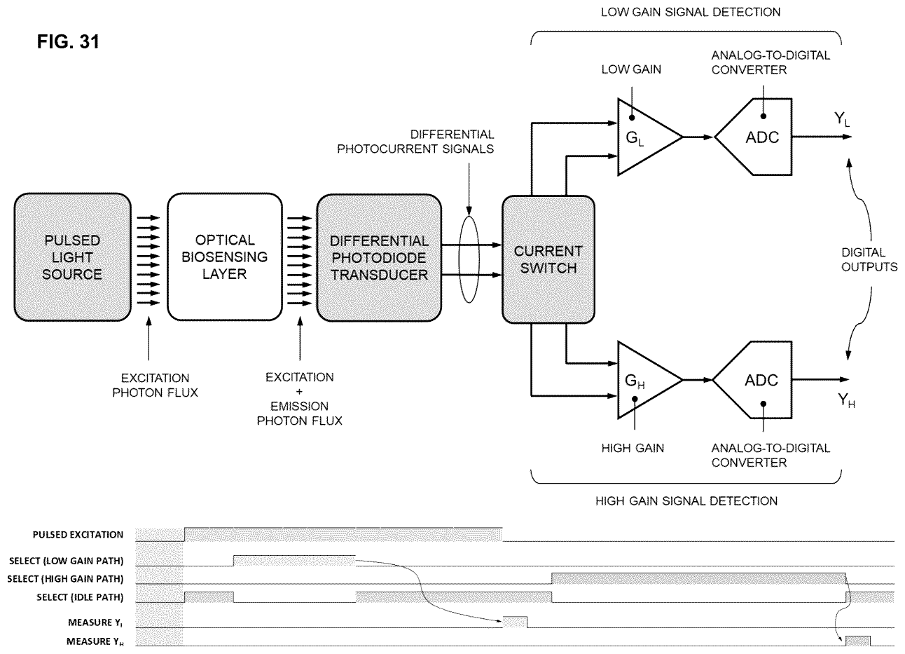

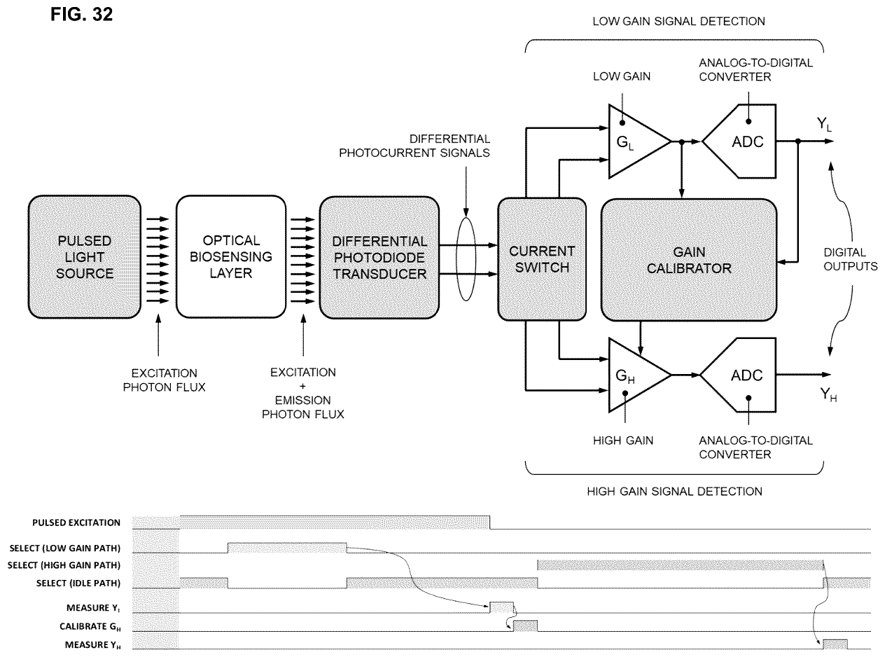

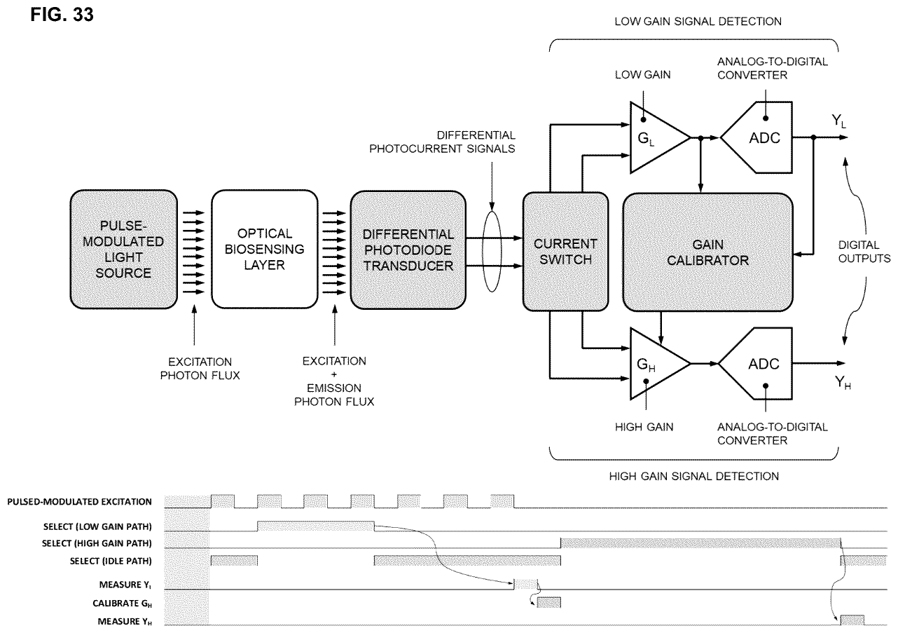

In some embodiments, this configuration may be called a “flip probe”. In some embodiments, a flip probe may be probe with both donor and acceptor that has four segments and two or more labels. They are sequentially: (i) a first segment which is a target region hybridizing segment of about 1 to 7 bases, (ii) a first label, (iii) a second segment which is a target region hybridizing segment of about 4 to 10 bases, (iv) a third region which is a target region hybridizing segment of about 4 to 9 bases, (v) a fourth segment of about 4 to 10 bases, and (vi) a second label. See FIG. 21 of U.S. Pat. No. 9,834,817, the content of which is incorporated by reference in its entirety. In some embodiments, the second segment and the fourth segment are complementary to each other such that in the absence of a target region of the analyte, the second segment and forth segment hybridize to position the first and second labels in close proximity to each other. In some embodiments, the first label is a donor, and the second label is an acceptor. In some embodiments, the first label is an acceptor, and the second label is a donor. In some embodiments, the fluorophore is near the 5′ end of the Flip Probe and is in close proximity to the label on the 3′ end of the Selector probe to enable FRET interactions when the probe is not bound to a target region. In some embodiments, the first segment of the Flip Probe is used to “space” the distance between the two labels for optimal FRET interactions.

In some embodiments, the probe may comprise an aptamer. As used herein, the term “aptamer” generally refers to a nucleic acid that has a specific binding affinity for a target molecule (e.g., a target analyte). Affinity interactions are a matter of degree. Accordingly, the term “specific binding affinity” of an aptamer for its target analyte means that the aptamer binds to its target analyte generally with a much higher degree of affinity than it binds to other nucleic acids in a test sample. In some embodiment, a probe may comprise an aptamer comprising a target-induced structural switching mode: A probe may comprise a first segment which is an aptamer for the target analyte and a second segment comprising a reporter (donor). The second segment of the probe may form a partial duplex with another small oligonucleotide modified with a quencher in the absence of the target. This configuration may bring the reporter (donor) and the acceptor into close proximity for maximum fluorescence energy transfer. When the target analyte is introduced, the aptamer segment (the first segment) prefers to form the aptamer-target analyte complex. The binding of the target analyte to the aptamer segment may change the structure of the probe and release the small oligonucleotide together with the acceptor, thereby triggering an increase of the signal and lifetime from the reporter since the acceptor is gone.

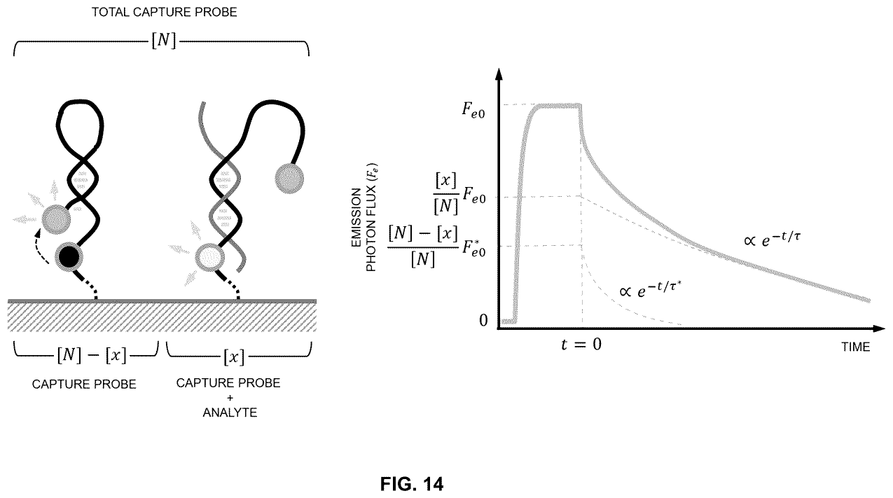

FIG. 14, shows the photon emission waveform of a biosensor that uses the method of FIG. 13. The total concentration of the probes at the surface is [N] and the captured analyte concentration is [x], making the concentration of free probes [N]-[x]. As shown, the emission waveform may consist of two exponentially decaying signals. As the surface captures more analytes, the observed decay may become more dominated by the exponential decay with lifetime of T. Therefore, by measuring the decay and computing both the amplitude of both exponential decays, one can estimate the ratio of [N] and [x] in that capturing spot. For example, the decay profiles of the capturing probes alone (in the absence of the analyte) and analyte-capturing probe duplex alone (in the presence of excess analytes to ensure substantially all capturing probe bound with the corresponding analytes) can be observed and measured to establish the boundaries of lifetimes for this construct.

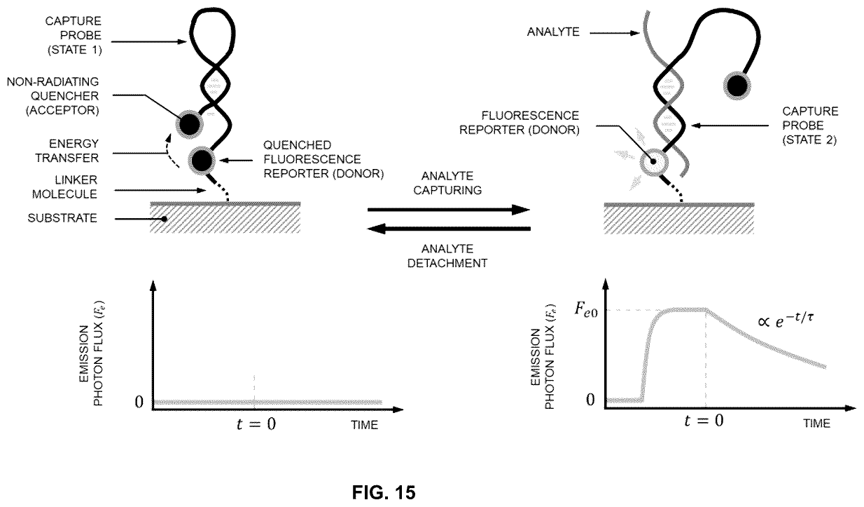

In FIG. 15, the sixth method of creating a time-resolved optical signal is shown. In this method, the capturing probe that is immobilized on the surface may comprise a FRET system with a fluorescence donor that has a lifetime of t and a quencher (non-radiating) acceptor. This probe, once excited with a photon flux pulse with wavelengths appropriate for its donor reporter, may emit a time-resolved signal either with lifetime of t or remain dark depending on the distance between its donor and acceptor. The structure of the probe may be designed such that in the absence of the analyte (State 1), the FRET system may be established, and no emission may occur. When the analyte is captured however, the donor and acceptor may move further from each other, and the FRET system is negated (State 2), and a signal is generated with a lifetime of T.

In some embodiment, the probe may contain both a fluorescent label and a quencher moiety. The term “quencher”, as used herein, generally refers to a moiety that interacts with the fluorescent label (donor) to modulate the amount of signal detected from the fluorescent label (donor). Typically, the quencher moiety decreases the amount of signal emitted by the fluorescent label when it is in close physical proximity to the fluorescent label (donor). Thus, in the open position, the unmatched region of the oligonucleotide is able to come into physical proximity to the fluorescent label (e.g., self-hybridized to form a hairpin structure) and quench the fluorescence of the fluorescent label; while in the closed position, the same region is duplexed with the target analyte and is physically separated from the fluorescent label, and no quenching effect is possible.

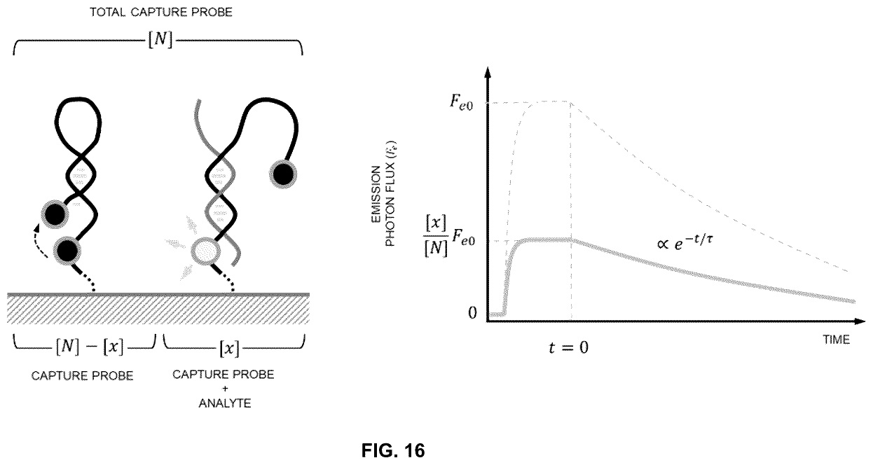

FIG. 16, shows the photon emission waveform of a biosensor that uses the method of FIG. 15. The total concentration of the probes at the surface is [N] and the captured analyte concentration is [x], making the concentration of free probes [N]-[x]. As shown, the emission waveform may consist of one exponentially decaying signal. As the surface captures more analytes, the amplitude of the signal may increase. Therefore, by measuring the amplitude of decaying signal with and without the analyte present, one can estimate both [N] and [x] in that capturing spot.

### Signal Detection and Feature Extraction

In this disclosure, there may be three (3) categories of quantitative measurements that are needed to analyze the signal and subsequently extract features such as lifetime and decaying amplitude. They are:
(1) Time measurements to know (i) when the excitation pulses are applied and (ii) at what time instance measurements are taken, are both critical in all embodiments of this disclosure. It is important to recognize that most of time-related parameters are set by the user and are fundamentally deterministic (e.g., excitation pulse width and integration times). However, once they are implemented in an optoelectronics detection hardware, they may become subject to offsets and/or drifts and/or probabilistic variations. Statistics of such uncertainties (mean, variance, probability distribution function, etc.) are to be known and/or properly measured and/or monitored to ensure that the analysis is performed with adequate time accuracy. The overall error/inaccuracy of timing measurement using the lifetime of the reporters (τ) as the benchmark (or unit) may be expected to be ≤τ×10⁻², and is preferred to be ≤τ×10⁻⁶. For example, if the lifetime of the reporter is 1 ms (10⁻³s), expected accuracy should be ≤10-5s, but preferably ≤10⁻⁹s.
(2) Excitation photon flux amplitude and spectrum measurements are important in all embodiments of this disclosure. The rationale behind this is that the emission signal may be a direct function of (or response to) these two parameters. Therefore, without knowing their values, quantitative time-resolved measurements may not be feasible. The excitation amplitude measurement preferred resolution is at least ≥8 bits (greater than 256 levels) and is preferred to be ≥12 bits (greater than 4096 levels). The wavelength resolution is at least ≤0.1 nm and is preferred to be ≤0.01 nm.
(3) Emission photon flux amplitude needs to be accurately measured to quantify the probe and analyte interaction and/or capturing. The emission amplitude may be measured with a resolution of ≥12 bits (greater 4,096 levels) and preferred to be with a resolution of ≥ 20 bits (greater than 106 levels).

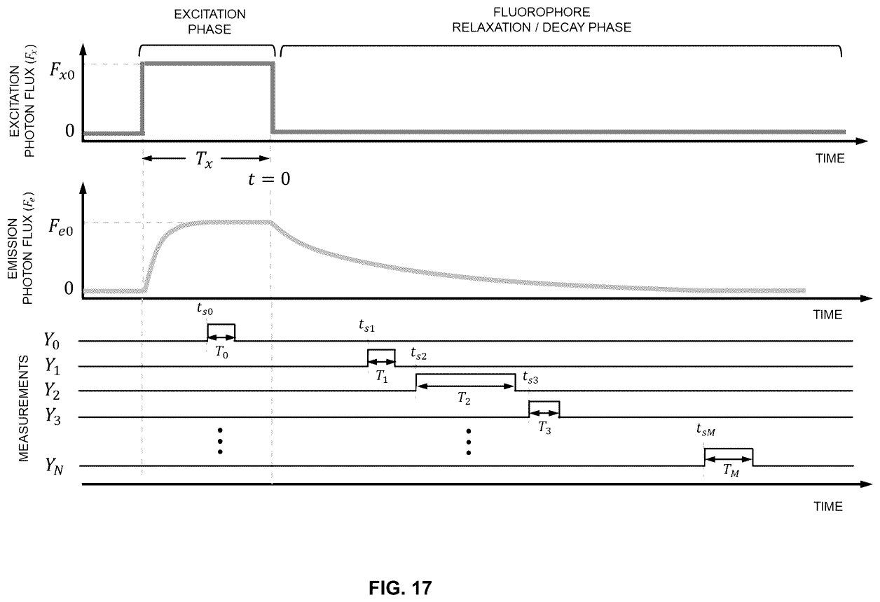

In FIG. 17, a generalized diagram of time-resolved measurements used in all embodiments of this disclosure is shown. An excitation pulse with a width of Tₓ is applied with a termination (end) time instance of t=0. The emission photon flux (response of the fluorescence reporters within the system) may start as soon as the excitation pulse is applied and increases until it reaches its steady state (equilibrium), assuming that the excitation pulse is long enough. The decaying emission photon flux that may include one or more exponentially decaying signals with different lifetimes may begin after the excitation pulse is terminated.

One or more measurements may be taken when the excitation pulse is applied to quantify the excitation photon flux. For example, the total photon flux observed in the system can be collected during the steady state of the emission signal starting at time t=tₛ₀ and ending at t=tₛ₀+T₀ (FIG. 17). The general equation for the measured value Yₚₕ₀, which is the total collected photons is equation (3):

$${Y}_{ph 0} = ( {k}_{x} {F}_{x 0} + {k}_{e} {F}_{e 0} ) {T}_{0} , \qquad ( 3 )$$

where kₓ and kₑ are the photon collection efficiency of the detector (photosensor) for those specific wavelengths and photon flux spatial distributions.

In a preferred embodiments of this disclosure, Yₚₕ₀ is measured to evaluate the excitation flux amplitude. Since Fₓ₀ »Fₑ₀ for most practical fluorescence biosensing systems, this measurement can be done without spectrally removing emission since that one can safely assume

$${Y}_{ph 0} \approx {k}_{x} {T}_{0} {F}_{x 0} . \qquad ( 4 )$$

To measure the emission photon flux, one or more measurements may be taken after the excitation pulse is terminated, i.e., t>0. As shown in FIG. 17, the values of such M measurements are denoted by Yₚₕ₁, Yₚₕ₂, . . . , Y_{phM}, each of which has a start time instance of tₛᵢ with integration duration of Tᵢ where i=1, 2, . . . , M. Using equation (2) and assuming that there is only one lifetime in the system, the following formulation for Yₚₕᵢ may be obtained in equation (5)

$${Y}_{phi} \approx {k}_{e} {τ F}_{e 0} ( {e}^{- \frac{{t}_{si}}{τ}} - {e}^{- \frac{{t}_{si} + {T}_{i}}{τ}} ) . \qquad ( 5 )$$

Now, equation (5) can be reformulated to consider multiple exponentials as well. Considering the system of FIG. 8 for example, Yₚₕᵢ becomes equation (6):

$${Y}_{phi} \approx {k}_{e} \{ {τ F}_{e 0} \frac{[ N ] - [ x ]}{[ N ]} ( {e}^{- \frac{{t}_{si}}{τ}} - {e}^{- \frac{{t}_{si} + {T}_{i}}{τ}} ) + {τ}^{*} {F}_{e 0}^{*} \frac{[ x ]}{[ N ]} ( {e}^{- \frac{{t}_{si}}{τ}} - {e}^{- \frac{{t}_{si} + {T}_{i}}{τ}} ) \} . \qquad ( 6 )$$

There are two important facts regarding equations (5) and (6) which are key to the data analysis for all embodiments of this disclosure. The first is that by selecting the appropriate M one can ensure that there can be enough measurements to estimate all the lifetimes in addition to the relative concentration of the probes with captured analytes vs. free probes (i.e., [x]/[N]). The second is that if there is one dominant long (slow) lifetime in the system, e.g., τ»τ* in (6), then one can select tₛᵢ's such that measurements from the system becomes only a function of the long (slow) lifetime. This significantly simplifies that data analysis. For example, if tₛᵢ »τ* and τ»τ* for the system in FIG. 8, Yₚₕᵢ becomes equation (7):

$${Y}_{phi} \approx {k}_{e} {τ F}_{e 0} \frac{[ N ] - [ x ]}{[ N ]} ( {e}^{- \frac{{t}_{si}}{τ}} - {e}^{- \frac{{t}_{si} + {T}_{i}}{τ}} ) . \qquad ( 7 )$$

### Optical Detection Sensor Array

All embodiments of this invention include an optical detection sensor array to perform the tasks that are described in FIG. 17 for all the different capturing spots (biosensing elements) within the array.

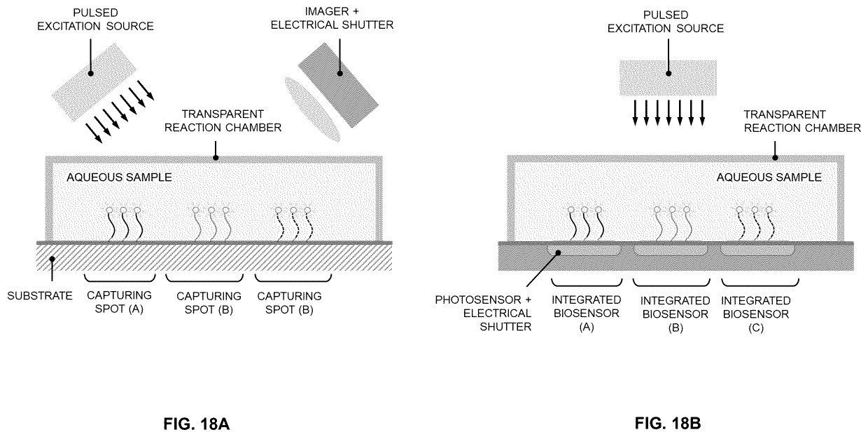

In certain embodiments of this disclosure, a focal plane imager (e.g., camera or microscope) with electrical or mechanical shutter may be used to take time-gated images from the array. This is shown in FIG. 18A. The special (2D) resolution of the imager and the number of pixels may depend on the number of capturing spots (biosensing elements). The time resolution of the shutter may follow the timing resolution that is needed for the adopted fluorescence lifetimes. In typical implementations, the time resolution may be ≥10 imaging pixels per capturing spot and may be preferred to have ≥104 imaging pixels per capturing spot.

In other embodiments of this invention an integrated biosensor array (sometimes referred to as an integrated biochip) may be used to implement the sensor array. As shown in FIG. 18B, in such a system the photosensors that have electrical shutters may be an integral part of the substrate and in intimate proximity of the capturing probes. Such systems are generally built using semiconductor manufacturing processes [6, 8, 18-20].

### Controls for Time-Resolved Biosensing

In a preferred embodiment, one may use certain biochemical constructs at a plurality of capturing spot of the biosensing array to act as the control for optical measurements and/or data analysis. The role of these control spots may be to provide real-time information regarding status of array and/or status of the linker and their stability and/or status of the fluorescence reporters and/or the lifetime of the fluorescence optical reporters and/or status of the excitation pulse.

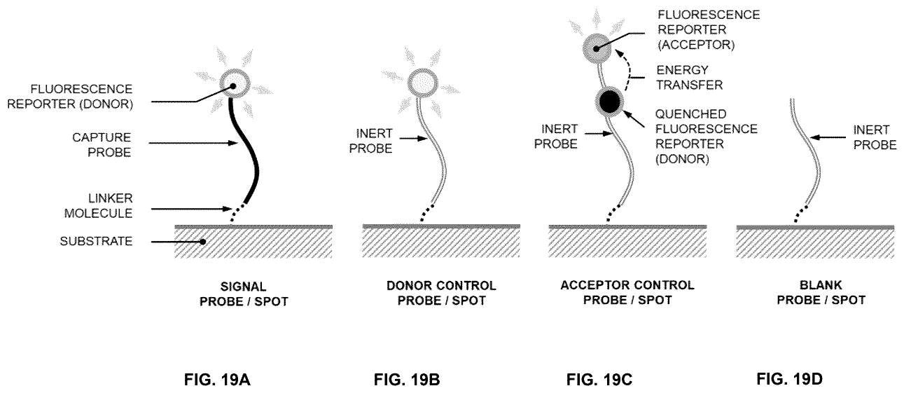

In FIGS. 19A-19D, a conventional capturing probe (FIG. 19A) and several example control probe constructs are shown (FIGS. 19B-D). A control for the donor fluorescence reporter is shown FIG. 19B. In this construct the capturing section of the probe may be deactivated or selected in such a way that it does not capture or interact with any analytes in the reaction chamber, i.e., capturing section is inert. In FIG. 19C, a control for the acceptor in the FRET configuration is shown and provides information regarding acceptor expected behavior and generated signal once analyte is captured. In FIG. 19D, an example blank spot (no signal) control is shown.

### Example Embodiments for Biomolecular Analysis

One can use the described time-resolved real-time biosensing embodiments of this disclosure to create a variety of biological analysis systems. In the following, a few examples are discussed.

### Real-Time Microarray

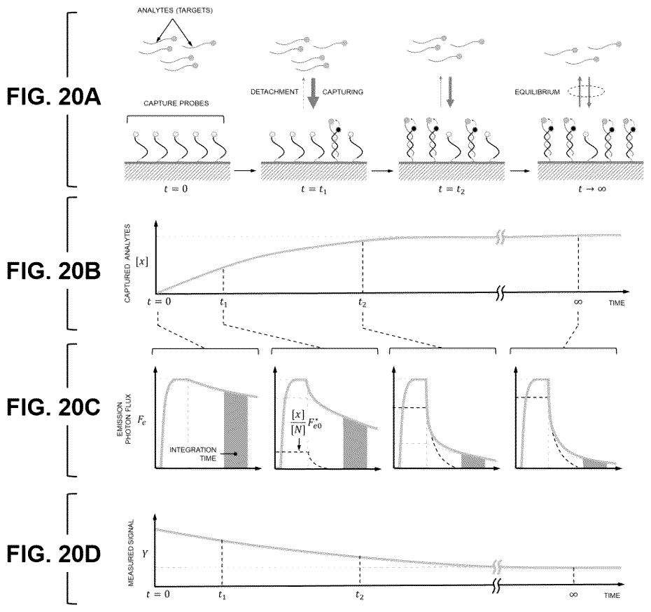

In FIGS. 20A-20D, a real-time microarray [7] that uses time-resolved optical reporters is shown, as an example. The premise is to estimate the concentration of a specific analyte by measuring its capturing kinetics as it interacts with a surface-immobilized probe. In this system, the temperature may be kept constant, and the time-resolved measurements may be taken as soon as the analytes are introduced in the reaction chamber. This approach has been previously reported using continuous fluorescence methods using FRET moieties [3, 21].

As shown in the example of FIGS. 20A-20C, the analyte capturing may comprise a fluorescence reporter (donor) with long lifetime and the probe may comprise a fluorescence reporter (acceptor) with short lifetime. The donor and the acceptor may be a FRET pair. When the analyte and the probe binds and forms a duplex, the donor and the acceptor, since placed in a close proximity, may form a FRET system and emits a fluorescence (acceptor) with short lifetime. This reduces the emission lifetime that originates from the formed duplex in the capturing spot. The signal may then be measured by integrating the emitted photons in fixed time intervals that include only the long lifetime decaying signal (e.g., the system described in equation (7)). In other words, the measurements may be done only at intervals in which the signal from short lifetime fluorescence reporter may be sufficiently diminished. As shown in FIG. 20C, the measured signal in this case may become proportional to the free capturing probes that have no analyte. Subsequently, these values may be used to estimate the concentration of the captured analyte. Finally, by monitoring captured analytes in real-time, one can observe the kinetics of capturing process and estimate the analyte concentration as described in the art [21]. Although this configuration uses a FRET pair as the reporters, other time-resolved optical reporter arrangements disclosed herein can also be used to perform this analysis.

### Solid-Phase Melt Curve Analysis

In FIGS. 21A-21D, a solid-phase melt curve analysis using the system for time-resolved optical detection disclosed in this disclosure is shown. The underlying premise is to measure the thermodynamic equilibrium characteristics of the analyte-probe duplex formation and denaturing and as a function of temperature [22-23].

As shown in the example of FIGS. 21A-21D, the analytes may comprise a fluorescence reporter (acceptor) with a short lifetime while the probes may comprise a fluorescence reporter (donor) with a long lifetime. The donor and the acceptor may be a FRET pair. When the analyte and the probe binds and forms a duplex, the donor and the acceptor, since placed in a close proximity, may form a FRET system. At low temperatures and in equilibrium, the analytes and the probes may all remain in the duplex form and emit photons with a short lifetime (the acceptor). When the temperature is increased gradually, the duplex slowly becomes more and more unstable, and eventually all duplexes are denatured. Now, the exact detachment profile and the melting temperature, defined as the temperature in which half of the probes are in the duplex form, may be a function of the reaction thermodynamics and duplex energies. Nonetheless, the detachment of the analyte in this system may move the donor and acceptor apart. They may result in an increase in the long lifetime emission (donor). This can then be measured, subsequently be used to create a melt curve and eventually be analyzed to estimate the thermodynamic characteristics of the reaction. Although this configuration uses a FRET pair as the reporters, other time-resolved optical reporter arrangements that are discussed in this disclosure can also be used to perform this melt curve analysis.

Real-Time Microarrays with Solid-Phase Melt Curve Analysis

The analyte quantification capabilities of real-time microarrays can be combined with solid-phase melt curve analysis to not only evaluate the concentration of analytes, but also evaluate their bond/duplex energies with the probe. This can be particularly informative when the nucleic acid target analytes are used and when one needs to not only detect the concentration, but also verify the nucleic sequence.

In FIGS. 22A-22C, an example embodiment of a system for quantification and sequence analysis for nucleic acids analyte targets is shown. Similar to previous example embodiments, the analyte may comprise short lifetime fluorescence reporters (acceptor) and the probes may comprise a long lifetime fluorescence reporter (donor). The donor and the acceptor may be a FRET pair. When the analyte and the probe binds and forms a duplex, the donor and the acceptor, since placed in a close proximity, may form a FRET system. Although this configuration uses a FRET pair as the reporters, other time-resolved optical reporter arrangements that are discussed in this invention can also be used to perform this analysis.

In FIG. 22A, probes A (5′-CCCTGAACTGGACCGT-3′) (SEQ ID NO: 1) and probes B (5′-CCCTGAAGTGGACCGT-3′) (SEQ ID NO: 2) are immobilized on capturing spots A and B, respectively. The probes A and probes B are depicted to capture nucleic acid target analyte A (5′-ACGGTCCAGTTCAGGG-3′) (SEQ ID NO: 3) and target analyte B (5′-ACGGTCCACTTCAGGG-3′) (SEQ ID NO: 4), respectively. Since the two probes and the unknown analytes to be tested may have different sequences; the bond strength and energy (AG's) to form the corresponding duplexes may be different. In this example, the analyte B-probe B duplex may have a more stable structure when compared to the analyte A-probe A duplex, i.e., −ΔG_{A}<-ΔG_{B}. In addition, the concentration of analyte A may be more than analyte B, i.e., [ΔG_{A}]> [ΔG_{B}]. Now, when the reaction chamber is subject to the temperature profile of FIG. 22B, the real-time measurement data of FIG. 22C may be measured using the approaches that are shown in FIGS. 21A-21D and FIGS. 20A-21D. As evident, after initial heating at 95° C. all the probes may be free, and the measured signals from both capturing spots may be maximized. However, as soon as the temperature is reduced and duplex formation is thermodynamically allowed (t=0), both analytes may start getting captured at each spot with capturing rates that are a function of their concentrations. In this case, analyte A may have a higher concentration when compared to analyte B, therefore the time-constant for analyte A capturing (TA) may become lower than the time constant for analyte B capturing (τ_{B}), i.e., τ_{A}<τ_{B} (NOTE: TA and τ_{B} notation should not be confused with fluorescence lifetime). After the capturing reactions reach equilibrium t=t₁, the system may be subject to a gradual heating ramp. Then both the capturing spots may show duplex destabilization. However, analyte-probe B duplex may show a higher melting temperature (T_{mB}) when compared to analyte-probe A duplex melting temperature (T_{mA}), i.e., T_{mB}>T_{mA}.

### Mutation Detection Using Differential Solid-Phase Melt Curve Analysis

Detecting small changes, i.e., mutations, in the genome sequence of living organisms is of great importance. Such mutations can be in the form of single nucleotide polymorphisms (SNPs) and/or insertions and/or deletions (indels). In FIGS. 23A-23D, the concept of a differential solid-phase melt curve analysis is shown. This method may detect small mutations in a sequence including SNPs and indels. The premise may be to use two or more dissimilar probes simultaneously and compare their kinetic and thermodynamic response to the target analyte that may or may not contain the targeted mutation [24-25].

The example probes in FIGS. 23A-23D may comprise a long lifetime fluorescence donor while the analytes may comprise a short lifetime acceptor. The nucleic acid sequence of at least one of the probes may be selected to match the sequence of the natural genome (wild-type) in the coordinate of the target mutation, while the sequence of one or other probes may be selected to match the mutated genome (mutant). Now, if a wild-type analyte is present, the wild-type probe may show faster kinetics and/or more stable analyte-probe duplex when compared to the mutant probe. Conversely, if a mutant analyte is present, the mutant probe may show faster kinetics and/or more stable analyte-probe duplex when compared to the wild-type probe. Therefore, by looking at the generated signal simultaneously and comparing them one can identify whether the mutant or the wild-type sequence is present.

In FIG. 23A, the possible reactions between the unknown target and wild-type and mutant probe-analyte DNA duplexes are depicted. In FIG. 23B, an example temperature profile for a real-time microarray plus differential melt curve analysis is shown in which the system may be initially reset by elevating the temperature to 95° C. to denature all DNA-DNA duplexes. Consequently, the temperature may be lowered to 30° C. to allow analyte capturing (DNA hybridization) and real-time time-resolved measurements. Finally, after the kinetics of the DNA hybridization reaches equilibrium, the system may be gradually heated to melt the duplexes while real-time time-resolved measurements continue. In FIG. 23C, example results for when the analyte is a wild-type are shown. In FIG. 23D, example results for when the analyte is mutant are shown. As evident, while the kinetics may look very similar, the melt temperatures can clearly differentiate between the mutant and wild-type sequences.

### Solid-Phase Quantitative Polymerase Chain Reaction

Real-time measurements using time-resolved optical reporters can be integrated into nucleic acid amplification testing (NAAT) systems. Examples amplification systems include polymerase chain reaction (PCR) [26], ligase chain reaction and Loop-Mediated Isothermal Amplification (LAMP) [28]. The premise is to use time-resolved optical reporters that are described in this disclosure as the reporter molecules for quantitative detection of the generated amplification products (amplicons). In other words, one can replace conventional continuous-wave fluorescence [6] or electrochemical reporters with time-resolved optical reporters use time-resolved detection systems to perform analytical measurements.

In FIG. 24, a specific embodiment of this disclosure is depicted that performs solid-phase quantitative polymerase chain reaction (SP-QPCR). The underlying principle of SP-QPCR is to take advantage of solid-phase (surface) DNA-DNA hybridization reactions to monitor solution-phase PCR in single plex (i.e., one amplification in the reaction tube) or multiplex (i.e., multiple amplification processes in one reaction). To do so, DNA oligonucleotide probes comprising long lifetime fluorescence reporters (donors) may be placed on the substrate. The sequences for these probes may be selected to match the amplicons that need to be monitored. Quantification may be carried out by measuring the solid-phase hybridization of the amplicons comprising short lifetime fluorescence reporters (acceptors) at every PCR cycle and in the annealing and/or extension phase and subsequently verifying the sequence of the generated amplicons at the completion of PCR process (end-point analysis) through a solid-phase melt curve analysis.

In a preferred embodiment of SP-PCR that use time-resolved optical reporters, inclusion of fluorescence reporters to the amplicon may be done by attaching the reporters to the PCR primers during their manufacturing process, for example, at their 5-end. Therefore, any amplicon that is created by the elongation of the primers by the polymerase enzyme may have fluorescence reporter at its 5′-end. In some embodiments, only one of the primers (either the forward primer or the reverse primer, but not both) is labeled with an acceptor reporter molecule. When the amplicons are labeled at the 5′-ends, the probes are labeled with the donor reporter molecules at their 3′-end and immobilized to the surface of the reaction chamber at their 5′-end.

In some embodiments, the probes are sequence complementary to one of the primers (either the forward primer or the reverse primer, but not both). Only this primer (either the forward primer or the reverse primer, but not both) bears acceptor reporter molecules on its 5′-ends. In other words, the other primer which is not sequence complementary to the probes does not bear an acceptor reporter molecule. Additionally, the probes are configured not to hybridize to the amplicons. The donor reporter molecule on the probe has a longer fluorescence emission lifetime than that of the acceptor reporter molecule on the analyte. At the start of the PCR process, all of the primers are available to bind to the probes and form the duplexes, thereby producing the strongest signals associated with the duplexes. If the donor and acceptor reporter molecules form a FRET pair, the duplexes emit characteristic emission fluorescence that can be recorded and analyzed according to the systems and methods disclosed herein. When the PCR amplification proceeds, more and more primers are incorporated into the amplicons such that the primers form fewer and fewer duplexes since the formed amplicons do not bind to the probes. For example, elongated part of the amplicons from the primers decreases the affinity of the amplicons to the probes. Time-resolved fluorescence signals provide real-time readings of signals and enable the estimation of the number of the probes in the duplex form or the free form and the ratio of duplexed probes to the free probes. The analysis leads to the estimation of the initial concentration of the analytes when the real-time time-resolved detection of the signal of the PCR reaction is completed.

In some embodiments, the probes are configured not to hybridize with the free primers and the probes are sequence complementary to one of the amplicons (either the one having the forward primer or the one having the reverse primer, but not both amplicons). The probes bearing the donor reporter molecules at their 3′-end are immobilized to the surface of the reaction chamber on their 5′-ends. The amplicons bear the acceptor reporter molecules close to their 5′-ends, for example, on one of the nucleic acids near the 3′-ends of the primer. The donor and acceptor reporter molecules form a FRET pair. The donor reporter molecule on the probe has a longer fluorescence emission lifetime than that of the acceptor reporter molecule on the analyte.

In some embodiments, the probes are configured not to hybridize with the free primers and the probes are sequence complementary to one of the amplicons (either the one having the forward primer or the one having the reverse primer, but not both amplicons). The probes are immobilized to the surface of the reaction chamber on their 3′-ends, and the donor reporter molecules are placed near their 3′-end, for example, attached to one of the nucleic acids near the 3′-ends of the probes. The amplicons bear the acceptor reporter molecules close to their 5′-ends, for example, on one of the nucleic acids near the 3′-ends of the primer. The donor and acceptor reporter molecules form a FRET pair. The donor reporter molecule on the probe has a longer fluorescence emission lifetime than that of the acceptor reporter molecule on the analyte.

### Computer Systems

The present disclosure provides computer systems that are programmed to implement methods of the disclosure. FIG. 25 shows a computer system 2501 that is programmed or otherwise configured to implement the methods described herein, e.g., methods for detecting a presence or absence of an analyte. The computer system 2501 can regulate various aspects of excitation light generation, acquisition of output signal, and signal processing of the present disclosure, such as, for example, generating pulsed excitation light for the detection of a presence or absence of an analyte. The computer system 2501 part of a system configured for detection of an analyte. The computer system may be integrated with the detection system. Alternatively, or in addition to, the computer system may be an external computer system coupled to the detection system via wired connection or wireless connection (e.g., Wi-Fi or Bluetooth connection).

The computer system 2501 includes a central processing unit (CPU, also “processor” and “computer processor” herein) 2505, which can be a single core or multi core processor, or a plurality of processors for parallel processing. The computer system 2501 also includes memory or memory location 2510 (e.g., random-access memory, read-only memory, flash memory), electronic storage unit 2515 (e.g., hard disk), communication interface 2520 (e.g., network adapter) for communicating with one or more other systems, and peripheral devices 2525, such as cache, other memory, data storage and/or electronic display adapters. The memory 2510, storage unit 2515, interface 2520 and peripheral devices 2525 are in communication with the CPU 2505 through a communication bus (solid lines), such as a motherboard. The storage unit 2515 can be a data storage unit (or data repository) for storing data. The computer system 2501 can be operatively coupled to a computer network (“network”) 2530 with the aid of the communication interface 2520. The network 2530 can be the Internet, an internet and/or extranet, or an intranet and/or extranet that is in communication with the Internet. The network 2530 in some cases is a telecommunication and/or data network. The network 2530 can include one or more computer servers, which can enable distributed computing, such as cloud computing. The network 2530, in some cases with the aid of the computer system 2501, can implement a peer-to-peer network, which may enable devices coupled to the computer system 2501 to behave as a client or a server.

The CPU 2505 can execute a sequence of machine-readable instructions, which can be embodied in a program or software. The instructions may be stored in a memory location, such as the memory 2510. The instructions can be directed to the CPU 2505, which can subsequently program or otherwise configure the CPU 2505 to implement methods of the present disclosure. Examples of operations performed by the CPU 2505 can include fetch, decode, execute, and writeback.

The CPU 2505 can be part of a circuit, such as an integrated circuit. One or more other components of the system 2501 can be included in the circuit. In some cases, the circuit is an application specific integrated circuit (ASIC).

The storage unit 2515 can store files, such as drivers, libraries and saved programs. The storage unit 2515 can store user data, e.g., user preferences and user programs. The computer system 2501 in some cases can include one or more additional data storage units that are external to the computer system 2501, such as located on a remote server that is in communication with the computer system 2501 through an intranet or the Internet.

The computer system 2501 can communicate with one or more remote computer systems through the network 2530. For instance, the computer system 2501 can communicate with a remote computer system of a user (e.g., laboratory technician, researcher, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2501 via the network 2530.

Methods as described herein can be implemented by way of machine (e.g., computer processor) executable code stored on an electronic storage location of the computer system 2501, such as, for example, on the memory 2510 or electronic storage unit 2515. The machine executable or machine readable code can be provided in the form of software. During use, the code can be executed by the processor 2505. In some cases, the code can be retrieved from the storage unit 2515 and stored on the memory 2510 for ready access by the processor 2505. In some situations, the electronic storage unit 2515 can be precluded, and machine-executable instructions are stored on memory 2510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 2501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2501 can include or be in communication with an electronic display 2535 that comprises a user interface (UI) 2540 for providing, for example, operating parameters of the system, system status, or outputs of methods described elsewhere herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2505. The algorithm can, for example, process output signals for determination of a presence or absence of an analyte.

### Systems and Devices for Time-Resolved Optical Detection

The systems, devices, and methods for time-resolved optical detection of the present disclosure may include complementary metal-oxide-semiconductor (CMOS) chips integrated into an optical sensor array with addressable locations. The analyte sensing system may include, but is not limited to, a sensor array, reaction chamber, excitation source, controllable fluidic system, temperature controller, heaters, reagents and reporter constructs, and a digital or computer system. The sensor array may be 2D array configured to detect analytes by interfacing a top surface (e.g., surface layer) with a solution containing or suspected of containing an analyte.

The reaction chamber may provide the interface between the sample fluid (e.g., a fluidic aqueous sample that includes the analytes) with the sensor array. The reaction chamber may have any volume usable for detection of an analyte. For example, the reaction chamber may have a volume from about 0.1 microliters (μL) to 10,000 μL. In another example, the reaction chamber may have a volume from about 1 μL to about 100 μL. The reaction chamber may include a plurality of inlets and outlets to permit interfacing with a controllable fluidic system.

The excitation source may introduce wavelength specific photon flux into the reaction chamber and toward the surface of the sensor array in a controlled and synchronized operation. The excitation source may comprise an optical light source that can create a wavelength selective photon flux with a controllable and time-varying amplitude. The light source may illuminate the sensing layer of the device and the coordinates in which signal transduction may take place. The excitation source center wavelength may be from about 200 nanometers (nm) to 1500 nm. In an example, the excitation source center wavelength may be from about 300 nm to 800 nm. The excitation source special span (e.g., bandwidth) may be from about 1 nm to 500 nm. In an example the bandwidth may be from about 10 nm to 100 nm. The excitation source photon flux may be directional and may be optically collimated. Alternatively, the excitation source may not be optically collimated. The excitation source peak output power may be from about 10 milliwatts (mW) to 100 watts (W). In an example, the excitation source peak output power may be from about 100 mW to 10 W. The excitation source may be capable of pulsing at a frequency of about 10 GHz (e.g., turning on and off in 0.1 nanoseconds (ns) to about 100 MHz (e.g., turning on and off in 0.01 microsecond (μs)), or at a frequency lower than 100 MHz. The excitation source may be capable of pulsing at a frequency of about 10 GHz, 9 GHz, 8 GHZ, 7 GHZ, 6 GHZ, 5 GHZ, 4 GHZ, 3 GHZ, 2 GHZ, 1 GHZ, 900 MHz, 800 MHz, 700 MHz, 600 MHz, 500 MHz, 400 MHz, 300 MHz, 200 MHz, 100 MHz. The excitation source may be capable of pulsing at a frequency of about 9 GHz to about 10 GHZ, about 8 GHz to about 9 GHZ, about 7 GHz to about 8 GHZ, about 6 GHz to about 7 GHz, about 5 GHz to about 6 GHZ, about 4 GHz to about 5 GHZ, about 3 GHz to about 4 GHZ, about 2 GHz to about 3 GHZ, about 1 GHz to about 2 GHz, about 900 MHz to about 1 GHZ, about 800 MHz to about 900 MHz, about 700 MHz to about 800 MHz, about 600 MHz to about 700 MHz, about 500 MHz to about 600 MHz, about 400 MHz to about 500 MHz, about 300 MHz to about 400 MHz, about 200 MHz to about 300 MHz, about 100 MHz to about 200 MHz, about 90 MHz to about 100 MHz, about 80 MHz to about 90 MHz.

The controllable fluidic system may be configured to direct fluid to or remove fluid from or may direct fluid to or remove fluid from the sensor array, including the sample or reagents, in a controlled and synchronized operation. Methods described herein may include using the controllable fluidic system to direct fluid to or from the reaction chamber. The controllable fluidic system may be used to execute the workflow and/or processes for detection and analysis of an analyte. The workflow and sequence of each fluidic operation may be selected based on the assaying method and may be, for example, flow-through and mono-directional or closed-tube. The controllable fluidic system may use fluidic components such as pumps, valves, and tubing to perform the workflow.

The temperature controller may be configured to set the temperature or may set the temperature of the reaction chamber. Methods may include using the temperature controller to set and maintain a specific temperature of the fluid of the reaction chamber or generate a temperature profile for heating or cooling. A temperature controller may include a feedback control system that measures the temperature, using temperature sensor with tin the sensor array or sensor devices coupled with the reaction chamber (e.g., a thermistor or thermocouple) and, based on the measured temperature, add or remove heat from the reaction chamber using heaters or thermal devices (e.g., Peltier devices or resistive heaters). The system may include a single heater or a plurality of heaters. The heater(s) may be integrated into the system or into the sensing array. In an example, the heater(s) are resistive-type heater(s). Temperature controllers may comprise heat sings for removing heat. Temperature controllers may have components within the sensor array or external to the sensor array. Temperature controllers may change the temperature of a substrate, reaction chamber, or sensor array. The rate of temperature change may be about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20° C. per second. The rate of temperature change can be at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20° C. per second. The rate of temperature change can be at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20° C. per second. Temperature controllers can change temperature at a linear rate (e.g., 5° C. per second). Alternatively, temperature controllers can change temperature at a non-linear rate. Temperature controllers can increase or decrease temperature.

Systems and devices for time-resolved optical detection described herein may include a light source, optical sensing layer (e.g., surface layer), filters, transducers, detection circuitry, or any combination thereof. FIG. 26 shows an example block diagram of a sensor system using fluorescent-based transductions methods. The system may include a light source that generates an excitation photon flux and directs the photon flux to a sensing layer, for example, an optical sensing layer. The optical sensing layer may comprise an interaction moiety (e.g., capturing probe) configured to bind or otherwise interact with the analyte to generate an optical signal in a biosensor configuration. The optical sensing layer may generate an emission photon flux that may be filtered using one or more optical filters or no filter at all. The optical filters may filter excitation and/or scattered light to prevent the light from reaching the optical transducer (photodetector). The photodetector may convert the optical signal to an electrical (e.g., photocurrent) signal. The electrical current may be directed to detection circuitry (e.g., gain, analog-to-digital converters, etc.) configured to generate a digital output signal.

A sensor in an array of sensors may include a photodiode transducer. The photodiode transducer may be a differential photodiode transducer. A differential photodiode transducer may be an optical transducer that comprises at least two photodiode elements (e.g., photodiodes). The differential photodiode transducer may include at least 2, 3, 4, 5, 6, 8, 10, or more photodiodes. In an example, the differential photodiode transducer comprises two photodiodes. The photodiodes may be identical or may be different. In an example, the photodiodes are identical. The first photodiode may be configured to receive and transduce incident photons to an electrical signal (e.g., electrons) from the array and the sample. The first photodiode may be a bright photodiode as it is configured to receive or as it receives incident photons from the light source and sample. A second photodiode may comprise an optical cover. The optical cover may be configured to reduce or block or may reduce or block incident photons from contacting the second photodiode. As such, the second photodiode may be a “dark” photodiode.

The cover may comprise one or more metals. The one or more metals may include Aluminum, Copper, Tungsten, or other metals that are used in the manufacturing of semiconductor devices. The optical cover may block incident photons independent of the wavelength of the photons. For example, the optical cover may comprise a cover configured to block or that blocks all or substantially all wavelengths of light. Alternatively, the optical cover may filter the incident photons to remove selected wavelengths of incident photons. The optical cover may block greater than or equal to about 50%, 60%, 70%, 80%, 90%, 95%, 97%, 98%, 99%, or more of the incident photons. In an example, the optical cover blocks greater than or equal to about 95% of the incident photons. In another example, the optical cover blocks greater than or equal to about 99% of the incident photons. The cover may be disposed between the dark photodiode and the surface layer. The cover may be the same size or shape as the dark photodiode. The cover may have a larger surface area than the dark photodiode to block or substantially block the dark photodiode from photon flux. The cover may have a surface area that is at least about 1%, 2%, 3%, 5%, 7%, 10%, 15%, 20%, 25%, or greater larger than a surface area of the dark photodiode.

The first (e.g., bright) and second (e.g., dark) photodiode may be disposed in spatial proximity of one another. The first and second photodiode may be disposed at the same or substantially the same depth away from the surface. Alternatively, the first and second photodiode may be disposed at different depth away from the surface. In an example, the first and second photodiode are disposed at the same or substantially the same depth away from the surface such that the observed photon flux of both photodiodes is the same or substantially the same for each photodiode. The first and second photodiodes may be disposed adjacent to one another. The first and second photodiodes may be separated by a distance of less than or equal to about 1 millimeter (mm), 0.8 mm, 0.6 mm, 0.4 mm, 0.2 mm, 0.1 mm, 80 micrometers (μm), 60 μm, 40 μm, 20 μm, 10 μm, 8 μm, 6 μm, 4 μm, 2 μm, 1 μm, 800 nanometers (nm), 600 nm, 400 nm, 200 nm, 100 nm, or less. The first and second photodiodes may be separated by a distance of greater than or equal to about 100 nm, 200 nm, 400 nm, 600 nm, 800 nm, 1 μm, 2 μm, 4 μm, 6 μm, 8 μm, 10 μm, 20 μm, 40 μm, 60 μm, 80 μm, 0.1 mm, 0.2 mm, 0.4 mm, 0.6 mm, 0.8 mm, 1 mm, or greater. The first and second photodiodes may be separated by a distance from about 100 nm to 200 nm, 100 nm to 400 nm, 100 nm to 600 nm, 100 nm to 800 nm, 100 nm to 1 μm, 100 nm to 2 μm, 100 nm to 4 μm, 100 nm to 6 μm, 100 nm to 8 μm, 100 nm to 10 μm, 100 nm to 20 μm, 100 nm to 40 μm, 100 nm to 60 μm, 100 nm to 80 μm, 100 nm to 0.1 mm, 100 nm to 0.2 mm, 100 nm to 0.4 mm, 100 nm to 0.4 mm, 100 nm to 0.6 mm, 100 nm to 0.8 mm, 100 nm to 1 mm, 200 nm to 400 nm, 200 nm to 600 nm, 200 nm to 800 nm, 200 nm to 1 μm, 200 nm to 2 μm, 200 nm to 4 μm, 200 nm to 6 μm, 200 nm to 8 μm, 200 nm to 10 μm, 200 nm to 20 μm, 200 nm to 40 μm, 200 nm to 60 μm, 200 nm to 80 μm, 200 nm to 0.1 mm, 200 nm to 0.2 mm, 200 nm to 0.4 mm, 200 nm to 0.4 mm, 200 nm to 0.6 mm, 200 nm to 0.8 mm, 200 nm to 1 mm, 400 nm to 600 nm, 400 nm to 800 nm, 400 nm to 1 μm, 400 nm to 2 μm, 400 nm to 4 μm, 400 nm to 6 μm, 400 nm to 8 μm, 400 nm to 10 μm, 400 nm to 20 μm, 400 nm to 40 μm, 400 nm to 60 μm, 400 nm to 80 μm, 400 nm to 0.1 mm, 400 nm to 0.2 mm, 400 nm to 0.4 mm, 400 nm to 0.4 mm, 400 nm to 0.6 mm, 400 nm to 0.8 mm, 400 nm to 1 mm, 600 nm to 800 nm, 600 nm to 1 μm, 600 nm to 2 μm, 600 nm to 4 μm, 600 nm to 6 μm, 600 nm to 8 μm, 600 nm to 10 μm, 600 nm to 20 μm, 600 nm to 40 μm, 600 nm to 60 μm, 600 nm to 80 μm, 600 nm to 0.1 mm, 600 nm to 0.2 mm, 600 nm to 0.4 mm, 600 nm to 0.4 mm, 600 nm to 0.6 mm, 600 nm to 0.8 mm, 600 nm to 1 mm, 800 nm to 1 μm, 800 nm to 2 μm, 800 nm to 4 μm, 800 nm to 6 μm, 800 nm to 8 μm, 800 nm to 10 μm, 800 nm to 20 μm, 800 nm to 40 μm, 800 nm to 60 μm, 800 nm to 80 μm, 800 nm to 0.1 mm, 800 nm to 0.2 mm, 800 nm to 0.4 mm, 800 nm to 0.4 mm, 800 nm to 0.6 mm, 800 nm to 0.8 mm, 800 nm to 1 mm, 1 μm to 2 μm, 1 μm to 4 μm, 1 μm to 6 μm, 1 μm to 8 μm, 1 μm to 10 μm, 1 μm to 20 μm, 1 μm to 40 μm, 1 μm to 60 μm, 1 μm to 80 μm, 1 μm to 0.1 mm, 1 μm to 0.2 mm, 1 μm to 0.4 mm, 1 μm to 0.4 mm, 1 μm to 0.6 mm, 1 μm to 0.8 mm, 1 μm to 1 mm, 2 μm to 4 μm, 2 μm to 6 μm, 2 μm to 8 μm, 2 μm to 10 μm, 2 μm to 20 μm, 2 μm to 40 μm, 2 μm to 60 μm, 2 μm to 80 μm, 2 μm to 0.1 mm, 2 μm to 0.2 mm, 2 μm to 0.4 mm, 2 μm to 0.4 mm, 2 μm to 0.6 mm, 2 μm to 0.8 mm, 2 μm to 1 mm, 4 μm to 6 μm, 4 μm to 8 μm, 4 μm to 10 μm, 4 μm to 20 μm, 4 μm to 40 μm, 4 μm to 60 μm, 4 μm to 80 μm, 4 μm to 0.1 mm, 4 μm to 0.2 mm, 4 μm to 0.4 mm, 4 μm to 0.4 mm, 4 μm to 0.6 mm, 4 μm to 0.8 mm, 4 μm to 1 mm, 6 μm to 8 μm, 6 μm to 10 μm, 6 μm to 20 μm, 6 μm to 40 μm, 6 μm to 60 μm, 6 μm to 80 μm, 6 μm to 0.1 mm, 6 μm to 0.2 mm, 6 μm to 0.4 mm, 6 μm to 0.4 mm, 6 μm to 0.6 mm, 6 μm to 0.8 mm, 6 μm to 1 mm, 8 μm to 10 μm, 8 μm to 20 μm, 8 μm to 40 μm, 8 μm to 60 μm, 8 μm to 80 μm, 8 μm to 0.1 mm, 8 μm to 0.2 mm, 8 μm to 0.4 mm, 8 μm to 0.4 mm, 8 μm to 0.6 mm, 8 μm to 0.8 mm, 8 μm to 1 mm, 10 μm to 20 μm, 10 μm to 40 μm, 10 μm to 60 μm, 10 μm to 80 μm, 10 μm to 0.1 mm, 10 μm to 0.2 mm, 10 μm to 0.4 mm, 10 μm to 0.4 mm, 10 μm to 0.6 mm, 10 μm to 0.8 mm, 10 μm to 1 mm, 20 μm to 40 μm, 20 μm to 60 μm, 20 μm to 80 μm, 20 μm to 0.1 mm, 20 μm to 0.2 mm, 20 μm to 0.4 mm, 20 μm to 0.4 mm, 20 μm to 0.6 mm, 20 μm to 0.8 mm, 20 μm to 1 mm, 40 μm to 60 μm, 40 μm to 80 μm, 40 μm to 0.1 mm, 40 μm to 0.2 mm, 40 μm to 0.4 mm, 40 μm to 0.4 mm, 40 μm to 0.6 mm, 40 μm to 0.8 mm, 40 μm to 1 mm, 60 μm to 80 μm, 60 μm to 0.1 mm, 60 μm to 0.2 mm, 60 μm to 0.4 mm, 60 μm to 0.4 mm, 60 μm to 0.6 mm, 60 μm to 0.8 mm, 60 μm to 1 mm, 80 μm to 0.1 mm, 80 μm to 0.2 mm, 80 μm to 0.4 mm, 80 μm to 0.4 mm, 80 μm to 0.6 mm, 80 μm to 0.8 mm, 80 μm to 1 mm, 0.1 mm to 0.2 mm, 0.1 mm to 0.4 mm, 0.1 mm to 0.4 mm, 0.1 mm to 0.6 mm, 0.1 mm to 0.8 mm, 0.1 mm to 1 mm, 0.2 mm to 0.4 mm, 0.2 mm to 0.4 mm, 0.2 mm to 0.6 mm, 0.2 mm to 0.8 mm, 0.2 mm to 1 mm, 0.4 mm to 0.6 mm, 0.4 mm to 0.8 mm, 0.4 mm to 1 mm, 0.6 mm to 0.8 mm, 0.6 mm to 1 mm, or 0.8 mm to 1 mm. In an example, the first and second photodiode are in the same plane and separated by a distance of about 100 nm to about 1 mm such that each photodiode observes the same excitation photon flux during the sensing process.

The differential photodiode transducer may be configured to generate or may generate two electrical outputs, one corresponding to the bright photodiode and one corresponding to the dark photodiode (e.g., covered photodiode).

A sensing array may include a plurality of individual sensors. An individual sensor may comprise a differential photodiode comprising a first and a second photodiode as described elsewhere herein. FIGS. 27A-27C show example differential photodiode transducers an example circuit schematic. FIG. 27A shows an example differential photodiode transducer with a bright photodiode disposed adjacent to a dark photodiode. The differential photodiode transducer may be disposed below a transparent layer. The dark photodiode may have a metal cover disposed between the light source and the dark photodiode. The bright photodiode and the dark photodiode may both generate and electrical output signal. FIG. 27B shows an example sensor array comprising a grid of alternating bright and dark photodiodes. The photodiodes shown are fabricated as a 2D square disposed under a transparent layer. Each dark photodiode may include a blocking cover disposed between the dark photodiode and the light source. Each sensor (e.g., pixel) may include a bright and dark photodiode and each bright photodiode and dark photodiode may generate an electrical output signal. FIG. 27C shows an alternative example configuration of a sensor array. The photodiodes may have a 2D bar or rectangular configuration, with alternating bright and dark photodiodes each configured to generate an electrical output. The dark photodiodes may each include a cover disposed between the dark photodiode and the light source. The example sensor arrays illustrated in FIGS. 27A-27C may be fabricated using a planar semiconductor manufacturing process. The layers and materials shown in FIGS. 27A-27C may be similar to those used in CMOS fabrication processes. In FIGS. 27A-27C D_{D} and D_{B} represent the electrical outputs of the dark photodiode and bright photodiode, respectively.

Systems for time-resolved optical detection may include an excitation light source. The excitation light source may be configured to deliver or may deliver a pulsed light as an excitation source. The pulsed light excitation source may permit time-resolved, also referred to as time-gated, fluorescence detection by applying a finite-time pulsed light to the sensor array. Emitted photons may be detected after the end of the pulse (e.g., in the absence of excitation light). Time-resolved detection may be compatible with a variety of different fluorophores and fluorescent reporters. See References 8 and 12. Example fluorophores may have long emission lifetimes, such as, for example metal chelates (e.g., lanthanide chelates). The pulsed light source may be a light emitted diode (LED), laser diode (LD), or non-solid state-based laser. The system may include triggering electrical circuitry to permit fast turn off of the excitation photon flux. Using time-resolved detection in combination with pulsed excitation light may permit the sensor array to detect an analyte without the use of emission filters. As such, the fluorescent-based detection system described herein may not use an emission filter for the detection of an analyte.

The method may include directing a series of pulses from the excitation source towards the surface layer for a set period of time, halting the pulses, and taking one or more measurements from the sensing array (e.g., from a single sensor in the array or from multiple sensors across the array). The set period of time (e.g., duration of time which the excitation light source is pulsed prior to taking a measurement) may be greater than or equal to about 10 ns to 100 ms. The series of pulses may include at least about 2, 4, 6, 8, 10, 15, 20, 30, 40, 50, 60, 80, 100, 1000 or more pulses. In an example, the series of pulses includes at least 10 pulses. An individual pulse may have a duration of less than or equal to about 10-1, 10-2, 10-3, 10-4, 10-5, 10-6, 10-7, 10-8, 10-9 or less. The duration of time that the light source is active may be the same as the duration of time that the light source is inactive during a series of pulses. Alternatively, or in addition to, the pulse duration may be greater than or less than the non-pulse duration (e.g., when the light source is off) during a series of pulses.

The systems, devices, and methods for time-resolved optical detection described herein may be used with any class of fluorophore as the reporter molecule for the sensing process. The optical signal may be generated by a fluorescent reporter molecule associated with the analyte of the immobilized capturing probe. A fluorophore may have a predefined and non-zero relaxation lifetime, τ_{F}. With select excitation pulsing and detection timing, measurements of the decaying emissions may be taken once the photon flux is halted. Fluorophores with longer fluorescence lifetimes may enable more options for excitation pulsing and detection timing, making sensing easier. Additionally, longer relaxation lifetimes may permit background autofluorescence from biological materials (e.g., endogenous fluorophores such as melanin, collagen, etc.) to be ignored as such materials may have lifetimes of less than 10 nanoseconds (ns). The fluorophore lifetime may be greater than or equal to about 10 ns, 20 ns, 30 ns, 40 ns, 50 ns, 60 ns, 80 ns, 100 ns, 150 ns, 200 ns, 300 ns, 400 ns, 500 ns, 600 ns, 700 ns, 800 ns, 900 ns, 1 microsecond (μs), 2 μs, 3 μs, 4 μs, 5 μs, 6 μs, 7 μs, 8 μs, 9, μs, 10 μs, 20 μs, 30 μs, 40 μs, 50 μs, 60 μs, 70 μs, 80 μs, 90 μs, 100 μs, 200 μs, 300 μs, 400 μs, 500 μs, 600 μs, 700 μs, 800 μs, 900 μs, 1 millisecond (ms) or greater. The fluorophore lifetime may be about 10 ns to about 20 ns, about 20 ns to about 30 ns, about 30 ns to about 40 ns, about 40 ns to about 50 ns, about 50 ns to about 60 ns, about 60 ns to about 80 ns, about 80 ns to about 100 ns, about 100 ns to about 150 ns, about 150 ns to about 200 ns, about 200 ns to about 300 ns, about 300 ns to about 400 ns, about 400 ns to about 500 ns, about 500 ns to about 600 ns, about 600 ns to about 700 ns, about 700 ns to about 800 ns, about 800 ns to about 900 ns, about 900 ns to about 1 microsecond (μs), about 1 μs to about 2 μs, about 2 μs to about 3 μs, about 3 μs to about 4 μs, about 4 μs to about 5 μs, about 5 μs to about 6 μs, about 6 μs to about 7 μs, about 7 μs to about 8 μs, about 8 μs to about 9, μs, about 9 μs to about 10 μs, about 10 μs to about 20 μs, about 20 us to about 30 μs, about 30 μs to about 40 μs, about 40 μs to about 50 μs, about 50 μs to about 60 μs, about 60 μs to about 70 μs, about 70 μs to about 80 μs, about 80 μs to about 90 μs, about 90 μs to about 100 μs, about 100 μs to about 200 μs, about 200 μs to about 300 μs, about 300 μs to about 400 μs, about 400 μs to about 500 μs, about 500 μs to about 600 μs, about 600 μs to about 700 μs, about 700 μs to about 800 μs, about 800 μs to about 900 μs, and about 900 μs to about 1 millisecond (ms). In an example, the fluorophore lifetime is greater than 100 ns. Non-limiting examples of fluorophores with fluorescent lifetimes of greater than 100 ns include organometallic complexes. In an example, the fluorophore lifetime is greater than 100 μs. Non-limiting examples of fluorophores with fluorescent lifetimes of greater than 100 μs include lanthanide chelates. The fluorescent reporter molecules may include donor molecules and acceptor molecules. In an example, both the donor molecule and the accepter molecule may be long lifetime fluorescent molecules. In another example, the donor molecule may be a long lifetime fluorophore and the acceptor molecule may be a shorter lifetime or non-radiating acceptor fluorophore. The donor molecule and the acceptor molecule may be bound or otherwise associated with the capturing probe and analyte, respectively, to permit real-time binding measurements. Alternatively, the donor molecule and the acceptor molecule may be bound or otherwise associated with the analyte and the capturing probe, respectively.

The system and corresponding methods for time-resolved optical detection may be configured for detection of emission signals of a single wavelength or multiple wavelengths (e.g., multi-color capabilities). Differentiating fluorophores may be permitted by differences in fluorescence lifetimes after excitation. In some examples, fluorophores may be reactive or conjugated dyes, nucleic acid dyes, fluorescent proteins, cell function dyes, or any combination thereof. Experimental designs for the multiplex detection of multiple fluorophores are possible without the use of emission and excitation filter sets. Accordingly, multiple fluorophores can be detected in a single experiment by the differential electrical output signals of the bright and dark photodiodes in the absence of excitation and emission filter sets. Individual species of fluorophores may be detected based on the differences in their decay rates. For example, metal chelate, such as Lanthanide chelates may be used as time-resolved fluorophores. In some cases, time-resolved fluorophores may act as molecular reporters in time-resolved assays either as a standalone reporter or an element (donor or acceptor) in a fluorescence energy transfer moiety. Examples include, but are not limited to, Forster Resonance Energy Transfer (FRET) technologies. The role of time-resolved fluorophores may include facilitating the generation of a specific time-resolved fluorescent signal that may be correlated to the presence or absence of a molecular reaction or presence or absence of a specific target analyte. Time-resolved fluorophores may be used as labels for specific target analytes, in applications where the targets may be chemically modified to incorporate a time-resolved fluorophore. Examples includes, but are not limited to, Northern blots, Southern blots, DNA microarrays, quantitative Polymerase Chain Reaction (PCR), digital PCR, and diagnostic assays. In microarrays and Norther blots, the mRNA target analyte may be converted into a fluorophore-labelled complementary DNA (cDNA), for example, through reverse transcription. In Southern blots, a fluorophore-labeled cDNA may be used to identify a target sequence. In quantitative PCR and digital PCR, the fluorophore may be incorporated into an amplified nucleic acid sequence or a primer sequence to demonstrate the accumulation of a target sequence. In a diagnostic assay, a device may be used to sequester target nucleic acids, and a fluorophore-labelled cDNA may be used for direct detection.

Time-resolved fluorophores may be used as labels for the detection of probes in a sandwich assay. Non-limiting examples include Western Blots, Enzyme-Linked Immunosorbent Assay (ELISA), Enzyme-Linked Immuno SPOT (ELISPOT), FluoroSpot assay, protein arrays, or any combination thereof. In sandwich assays, the time-resolved fluorophores may be used as a direct method for detection, in which the fluorophore is conjugated to a primary detection antibody. Alternatively, or in addition to, the time-resolved fluorophore may be used as an indirect method for detection, for example, the fluorophore may be conjugated to a secondary antibody. ELISPOT assays may be used to quantitatively measure the frequency of cytokine secretion for a single cell. The ELISASPOT assay may be a form of immunostaining that uses antibodies to detect an analyte, including but not limited to, any biological or chemical substance (e.g., protein analytes or chemical analytes). FluoroSpot assays may use fluorescence to analyze multiple analytes, for example, by detecting the secretion of more than one type of protein or other analytes.

Time-resolved fluorophores may be used as labels in cell sorting, counting, or detecting methods. An example may be flow cytometry, in chick cells may be labeled with a fluorophore. For example, cells may be sorted and counted by their fluorescence profiles. Alternatively, or in addition to, the specific cellular characteristics or functions may be identified by their fluorescence profiles.

Time-resolved fluorophores may be used in application where solid-phase and immobilized capturing probes are labeled. An example may include inverse fluorophore assays. Time-resolved fluorophores may be used in assays in which chemical reactions may be monitored while a target analyte is introduced a reacting reagent. The target molecule or the reacting reagent may include time-resolved fluorophores. Examples may include, but are not limited to, Sanger Sequencing and Next Generation Sequencing (NGS) assays such as sequence-by-synthesis (SBS), and pyrosequencing.

The fluorescent-based sensing device may further include a current switch. A current switch may divert the output current, I₀, from the photodiode transducer to dissimilar current detection paths. For example, the current switch may divert the output current from the photodiode transducer to at least 2, 3, 4, 5, 6, 7, 8, 9, 10, or more detection paths. In an example, the current switch may direct the output current to at least two different detection paths. The first detection path may be a high gain detection path and the second detection path may be a low gain detection path. The current switch may direct the output current to the high gain detection path when the excitation light source is off (e.g., when excitation photon flux is zero). The current switch may direct the output current to the low gain detection path when the light pulse is active (e.g., when excitation photon flux is greater than zero). In an example, the sensing device does not include an emission filter and so, when the photon flux is active, the output signal of the differential photodiode may be dominated by the excitation source. This approach may not be usable with a sensing system that includes emission filters as the excitation photon flux is blocked by the emission filter, which may make the low gain measurements non-informative.

Example current switches and corresponding timing diagram are shown in FIG. 28. FIG. 28 illustrates an example system for time-resolved optical detection including a photodiode transducer connected to circuitry including three possible current pathways, such as a low gain signal detection path with outputs represented by S_{LP} and S_{LN}, high gain signal detection path with outputs represented by S_{HP} and S_{HN}, and idle path with may be terminated to bias voltage VB. As shown in the timing diagram, the low gain path, φ₁, may be activated to measure the excitation photon flux and the high gain path, $2, may be activated between a timepoint t₁ and t₂ to measure the emission signal. When neither the low gain nor high gain paths are activated, the system may be connected to an idle path φ₃. An alternative current switch configuration is shown in FIG. 29. The example current switch includes chopper stabilization switches which may be configured to suppress or reduce offset current, unbalanced charge injection, or both.

The sensor array may include a plurality of sensors (e.g., a plurality of pixels). The sensor array may include greater than or equal to about 10, 10², 10³, 10⁴, 10⁵, 10⁶, 10⁷, 10⁸, 10⁹ or more sensors (e.g., pixels). The sensor array may include from about 10 to 10², 10 to 10³, 10 to 10⁴, 10 to 10⁵, 10 to 10⁶, 10 to 10⁷, 10 to 10⁸, 10 to 10⁹, 10², to 10³, 10² to 10⁴, 10² to 10⁵, 10² to 10⁶, 10² to 10⁷, 10² to 10⁸, 10² to 10⁹, 10³ to 10⁴, 10³ to 10⁵, 10³ to 10⁶, 10³ to 10⁷, 10³ to 10⁸, 10³ to 10⁹, 10⁴ to 10⁵, 10⁴ to 10⁶, 10⁴ to 10⁷, 10⁴ to 10⁸, 10⁴ to 10⁹, 10⁵ to 10⁶, 10⁵ to 10⁷, 10⁵ to 10⁸, 10⁵ to 10⁹, 10⁶ to 10⁷, 10⁶ to 10⁸, 10⁶ to 10⁹, 10⁷ to 10⁸, 10⁷ to 10⁹, or 10⁸ to 10⁹ sensors. The sensors may be evenly spaced or the spacing of the sensors may vary across the sensor array. A sensor may be disposed adjacent to another sensor. A sensor may be separated from another sensor by a distance. The distance separating two sensors may be less than or equal to about 1 mm, 0.8 mm, 0.6 mm, 0.4 mm, 0.2 mm, 0.1 mm, 80 micrometers (μm), 60 μm, 40 μm, 20 μm, 10 μm, 8 μm, 6 μm, 4 μm, 2 μm, 1 μm, 0.5 μm, 0.2 μm, 0.1 μm, or less. The distance separating two sensors may be greater than or equal to about 0.1 μm, 0.2 μm, 0.5 μm, 1 μm, 2 μm, 4 μm, 6 μm, 8 μm, 10 μm, 20 μm, 40 μm, 60 μm, 80 μm, 0.1 mm, 0.2 mm, 0.4 mm, 0.6 mm, 0.8 mm, 1 mm, or greater. The distance separating two sensors may be from about 0.1 μm to 0.2 μm, 0.1 μm to 0.4 μm, 0.1 μm to 0.6 μm, 0.1 μm to 0.8 μm, 0.1 μm to 1 μm, 0.1 μm to 2 μm, 0.1 μm to 4 μm, 0.1 μm to 6 μm, 0.1 μm to 8 μm, 0.1 μm to 10 μm, 0.1 μm to 20 μm, 0.1 μm to 40 μm, 0.1 μm to 60 μm, 0.1 μm to 80 μm, 0.1 μm to 0.1 mm, 0.1 μm to 0.2 mm, 0.1 μm to 0.4 mm, 0.1 μm to 0.6 mm, 0.1 μm to 0.8 mm, 0.1 μm to 1 mm, 0.2 μm to 0.4 μm, 0.2 μm to 0.6 μm, 0.2 μm to 0.8 μm, 0.2 μm to 1 μm, 0.2 μm to 2 μm, 0.2 μm to 4 μm, 0.2 μm to 6 μm, 0.2 μm to 8 μm, 0.2 μm to 10 μm, 0.2 μm to 20 μm, 0.2 μm to 40 μm, 0.2 μm to 60 μm, 0.2 μm to 80 μm, 0.2 μm to 0.1 mm, 0.2 μm to 0.2 mm, 0.2 μm to 0.4 mm, 0.2 μm to 0.6 mm, 0.2 μm to 0.8 mm, 0.2 μm to 1 mm, 0.4 μm to 0.6 μm, 0.4 μm to 0.8 μm, 0.4 μm to 1 μm, 0.4 μm to 2 μm, 0.4 μm to 4 μm, 0.4 μm to 6 μm, 0.4 μm to 8 μm, 0.4 μm to 10 μm, 0.4 μm to 20 μm, 0.4 μm to 40 μm, 0.4 μm to 60 μm, 0.4 μm to 80 μm, 0.4 μm to 0.1 mm, 0.4 μm to 0.2 mm, 0.4 μm to 0.4 mm, 0.4 μm to 0.6 mm, 0.4 μm to 0.8 mm, 0.4 μm to 1 mm, 0.6 μm to 0.8 μm, 0.6 μm to 1 μm, 0.6 μm to 2 μm, 0.6 μm to 4 μm, 0.6 μm to 6 μm, 0.6 μm to 8 μm, 0.6 μm to 10 μm, 0.6 μm to 20 μm, 0.6 μm to 40 μm, 0.6 μm to 60 μm, 0.6 μm to 80 μm, 0.6 μm to 0.1 mm, 0.6 μm to 0.2 mm, 0.6 μm to 0.4 mm, 0.6 μm to 0.6 mm, 0.6 μm to 0.8 mm, 0.6 μm to 1 mm, 0.8 μm to 1 μm, 0.8 μm to 2 μm, 0.8 μm to 4 μm, 0.8 μm to 6 μm, 0.8 μm to 8 μm, 0.8 μm to 10 μm, 0.8 μm to 20 μm, 0.8 μm to 40 μm, 0.8 μm to 60 μm, 0.8 μm to 80 μm, 0.8 μm to 0.1 mm, 0.8 μm to 0.2 mm, 0.8 μm to 0.4 mm, 0.8 μm to 0.6 mm, 0.8 μm to 0.8 mm, 0.8 μm to 1 mm, 1 μm to 2 μm, 1 μm to 4 μm, 1 μm to 6 μm, 1 μm to 8 μm, 1 μm to 10 μm, 1 μm to 20 μm, 1 μm to 40 μm, 1 μm to 60 μm, 1 μm to 80 μm, 1 μm to 0.1 mm, 1 μm to 0.2 mm, 1 μm to 0.4 mm, 1 μm to 0.6 mm, 1 μm to 0.8 mm, 1 μm to 1 mm, 2 μm to 4 μm, 2 μm to 6 μm, 2 μm to 8 μm, 2 μm to 10 μm, 2 μm to 20 μm, 2 μm to 40 μm, 2 μm to 60 μm, 2 μm to 80 μm, 2 μm to 0.1 mm, 2 μm to 0.2 mm, 2 μm to 0.4 mm, 2 μm to 0.6 mm, 2 μm to 0.8 mm, 2 μm to 1 mm, 4 μm to 6 μm, 4 μm to 8 μm, 4 μm to 10 μm, 4 μm to 20 μm, 4 μm to 40 μm, 4 μm to 60 μm, 4 μm to 80 μm, 4 μm to 0.1 mm, 4 μm to 0.2 mm, 4 μm to 0.4 mm, 4 μm to 0.6 mm, 4 μm to 0.8 mm, 4 μm to 1 mm, 6 μm to 8 μm, 6 μm to 10 μm, 6 μm to 20 μm, 6 μm to 40 μm, 6 μm to 60 μm, 6 μm to 80 μm, 6 μm to 0.1 mm, 6 μm to 0.2 mm, 6 μm to 0.4 mm, 6 μm to 0.6 mm, 6 μm to 0.8 mm, 6 μm to 1 mm, 8 μm to 10 μm, 8 μm to 20 μm, 8 μm to 40 μm, 8 μm to 60 μm, 8 μm to 80 μm, 8 μm to 0.1 mm, 8 μm to 0.2 mm, 8 μm to 0.4 mm, 8 μm to 0.6 mm, 8 μm to 0.8 mm, 8 μm to 1 mm, 10 μm to 20 μm, 10 μm to 40 μm, 10 μm to 60 μm, 10 μm to 80 μm, 10 μm to 0.1 mm, 10 μm to 0.2 mm, 10 μm to 0.4 mm, 10 μm to 0.6 mm, 10 μm to 0.8 mm, 10 μm to 1 mm, 20 μm to 40 μm, 20 μm to 60 μm, 20 μm to 80 μm, 20 μm to 0.1 mm, 20 μm to 0.2 mm, 20 μm to 0.4 mm, 20 μm to 0.6 mm, 20 μm to 0.8 mm, 20 μm to 1 mm, 40 μm to 60 μm, 40 μm to 80 μm, 40 μm to 0.1 mm, 40 μm to 0.2 mm, 40 μm to 0.4 mm, 40 μm to 0.6 mm, 40 μm to 0.8 mm, 40 μm to 1 mm, 60 μm to 80 μm, 60 μm to 0.1 mm, 60 μm to 0.2 mm, 60 μm to 0.4 mm, 60 μm to 0.6 mm, 60 μm to 0.8 mm, 60 μm to 1 mm, 80 μm to 0.1 mm, 80 μm to 0.2 mm, 80 μm to 0.4 mm, 80 μm to 0.6 mm, 80 μm to 0.8 mm, 80 μm to 1 mm, 0.1 mm to 0.2 mm, 0.1 mm to 0.4 mm, 0.1 mm to 0.6 mm, 0.1 mm to 0.8 mm, 0.1 mm to 1 mm, 0.2 mm to 0.4 mm, 0.2 mm to 0.6 mm, 0.2 mm to 0.8 mm, 0.2 mm to 1 mm, 0.4 mm to 0.6 mm, 0.4 mm to 0.8 mm, 0.4 mm to 1 mm, 0.6 mm to 0.8 mm, 0.6 mm to 1 mm, or 0.8 mm to 1 mm. In an example, a distance between any two sensors is from about 0.1 μm to 1 mm. In another example, a distance between any two sensors is from about 20 μm and 200 μm.

FIG. 30 shows an example top view and cross-sectional view of an example differential fluorescence-based sensor system for time-resolved optical detection. The system may include a 2D array of sensors. A sensor of the sensor array may include a surface layer comprising a capturing probe immobilized thereon. Each sensor may be associated with or in sensing communication with a single type of capturing probe configured to selectively bind a single analyte. Alternatively, a sensor may be associated with or in sensing communication with multiple types of capturing probes, for example, at least 1, 2, 3, 4, 5, 6, 8, 10, or more types of capturing probes configured to selectively bind at least 1, 2, 3, 4, 5, 6, 8, 10, or more different analytes. Each sensor in the sensor array may be associated with or in sensing communication (e.g., optical communication with the photodiode transducer of the sensor) with a different type of capturing probe or multiple sensors may be associated with or in sensing communication with the same type of capturing probe (e.g., to permit duplicate readings across the sensor array). In an example, each sensor is associated with or in sensing communication (e.g., optical communication with the photodiode transducer of the sensor) with a single type of capturing probe such that each sensor detects a different analyte. In another example, at least 2, 3, 4, 5, or more sensors are associated with the same capturing probe such that the sensor array generates at least 2, 3, 4, 5, or more duplicate readings across the sensor array for an analyte.

The sensing array may include a sensing layer (e.g., surface layer). The surface layer may be integrated with the sensing array and disposed on surface of the sensing array such that the surface layer is configured to contact or contact an aqueous solution comprising or suspected of comprising the analyte. The sensing layer may include an organic layer that may be created on top of the sensing array and configured to interface with a reaction chamber. The sensing layer may provide addressable locations comprising capturing probes disposed on top of individual sensors and permit, by transduction of an optical signal to an electrical signal, capturing and detection of an analyte. The sensing layer may be generated by various methods. For example, capturing probes or probe structures may be physically printed, immobilized, spotted, or chemically synthesized on the surface layer. In some examples, the capturing probes may be randomly distributed within the 2D array surface and then identified prior to detecting the targets by alternative approaches. For example, the surface layer (e.g., silicon oxide or silicon nitride surface) of the sensor array may be chemically modified with linkers or thin film structures to permit capturing probe attachment.

The capturing probes may be immobilized on a surface layer of the sensor array. The sensing array may include a single type of capturing probe or a plurality of different types of capturing probes. The capturing probes may be configured or selected to bind or interact with a targeted analyte. The capturing probes may be immobilized and spatially arranged such that they are disposed in optical communication of an individual sensor. The capturing probes may be disposed at independently or individually addressable locations on a solid surface of the sensing array. The capturing probes may be immobilized using a covalent or non-covalent linker. In an example, the capturing probes are immobilized using a covalent linker. In another example, the capturing probes are immobilized using a non-covalent linker. In another example, the capturing probes are immobilized using both covalent and non-covalent linkers. The linkers may be provided on the surface layer in excess of the number of capturing probes. The surface layer may include 10%, 20%, 30%, 40%, 50%, or more linkers than capturing probes. Unoccupied linkers (e.g., those not coupled to a capturing probe) may be chemical termination to prevent or reduce interaction or interference during sensing of an analyte.

As shown in FIG. 30, a differential photodiode transducer and cover for dark photodiode may be embedded in a silicon substrate of a CMOS device. The sensor array may further comprise isolators disposed between individual sensors. The isolators may be embedded in the silicon substrate and may be configured to optically isolate a sensor from another sensor. Alternatively, or in addition to, the isolators may be configured as optical pipes that direct optical signal from the surface layer to the photodiode transducer. The system for time-resolved optical detection may further include a reaction chamber disposed adjacent to the surface layer of the sensor array. The reaction chamber may be configured to hold a solution comprising the analyte such that the analyte may be in contact with the capturing probes immobilized on the surface layer.

FIG. 31 shows a block diagram of an example detection circuit integrated with each sensor (e.g., pixel), an example timing diagram for the system. The system for time-resolved optical detection may include a photodiode transducer, such as a differential photodiode transducer, a surface layer configured to permit an analyte to interact with a capturing probe, an excitation source configured to generate a pulsed excitation beam, and a current switch. The current switch may have three different detection paths, including an idle detection path (not shown). The differential photodiode transducer may produce two digital outputs, YL and YH, corresponding to the output of the low gain and high gain outputs, respectively. The system shown in FIG. 31 may be used to estimate fluorophore concentration,

FIG. 32 shows a block diagram of an example detection circuit integrated within the sensor, and timing diagram for the system. The illustrated system for time-resolved optical detection includes a differential photodiode transducer, a surface layer configured for sensing, a light source configured to direct pulsed excitation light to the surface layer, a current switch with at least two separate detection paths, and a calibration block. The calibration block may be configured to adjust the gain of the high gain path according to the excitation photon flux estimated in the low gain detection path. As shown in FIG. 33, the example detection circuit may be used with a pulse-modulated excitation source.

### EXAMPLES

### Example 1-Real-Time Microarray on the Device for Time-Resolved Optical Detection

An real-time microarray is constructed by immobilizing a plurality of capturing probes at an identifiable capturing spot. Each of the plurality of capturing probes bears a donor reporter molecules and is configured to hybridize with an analyte. The configurations of the real-time microarray for the time-resolved optical detection system are shown in FIG. 20A. Att=0, all capturing probes are free. Then the analytes labeled with fluorescence acceptors are added to the reaction chamber. The analytes interact with the capturing probes and start forming duplexes with the capturing probes over time (at t=t₁ and t₂, etc.) until reach an equilibrium between the free-flowing analytes and the captured analytes. See FIGS. 20A and 20B. The fluorescence donor reporter and the fluorescence acceptor reporter form a FRET pair. The fluorescence donor reporter on the probe has a longer fluorescence emission lifetime than that of the fluorescence acceptor on the analyte. Thus, the signals coming from the capturing spot with immobilized capturing probe-analyte duplex has a reduced emission lifetime relative to the free capturing probe on the same capturing spot. See FIG. 20C. A light source is used to radiate the reaction chamber for a first time period, then turns off for a second time period. An interval to record the emission signals from the capturing spot is selected to capture signals representing the remaining free capturing probe because signals from the duplex has a shorter emission lifetime and decreases more rapidly than those having a longer emission lifetime. As shown in FIGS. 20C and 20D, the amplitude and the fluorescence emission halftime of the detected signal at the chosen recording interval correspond to the changes of the concentration of the free capturing probes and/or the ratio of free capturing probes vs. capturing probe-analyte duplexes. The donor reporter molecule is Compound 4 and the acceptor reporter molecule is TMR. They are linked to the ends of the capturing probe and the analyte, respectively.

### Example 2-Solid-Phase Melt Curve Analysis

An array of capturing probes bearing donor reporter molecules at an independently addressable capturing spot is used to measure a sold-phase melt curve of analytes bearing acceptor reporter molecules. The probes and the analytes can form duplexes. The donor reporter on the probe has a longer fluorescence emission lifetime than that of the acceptor reporter molecule on the analyte. Thus, the signals coming from the capturing spot with immobilized capturing probe-analyte duplex has a reduced emission lifetime relative to the free capturing probe on the same capturing spot.

The configuration of this array is similar to that in Experiment 1. In melt curve analysis, the analytes and the capturing probes are allowed to interact with each other and form duplexes at 30° C. See FIG. 21A. Then the temperature of the reaction chamber is raised gradually from 30° C. to 95° C. according to a preset schedule. At each of the selected temperatures (e.g., T=30° C., . . . 45° C., . . . 60° C., 65° C., . . . 80° C., etc.), (i) the capturing probes and the analytes are allowed to reach an equilibrium at that temperature; (ii) the reaction chamber is radiated by the light source for a first time interval followed by turning off the light source; and (iii) the emitted fluorescence signals from the independently addressable capturing spot are recorded are recorded at selected time intervals after the light source is turned off. See FIGS. 21B-21C. The higher the temperature of the reaction chamber, the more dissociation of the capturing probe-analyte duplex. The ensuing changes of the signals at difference temperatures as shown in FIG. 21C. Shown in FIGS. 21C-21D are the recorded amplitude of the detected signals at selected temperatures, reflecting the changes of the concentration of the free capturing probes and/or the ratio of free capturing probes vs. capturing probe-analyte duplexes. The higher the temperature, the fewer the duplex forms of the capturing probes (and the more the free capturing probes), the longer the fluorescence emission lifetime of the detected signals. A first curve of measured fluorescence emission lifetimes vs. temperature is plotted using the measured data. The first curve is used to estimate/calculate the melting temperature defined as the temperature in which half of the probes are in the duplex form and the other half are in the free form. The first curve is then converted into a second curve, a percentage of duplexes remaining (or percentage of free capturing probes formed from the original duplexes) vs. temperature curve. The second curve provides another estimate/calculation for the melting temperature. The estimated melting temperature further provides information about the analytes captured by the capturing probes. The donor reporter molecule is Compound 4 and the acceptor reporter molecule is TMR. They are linked to the ends of the capturing probe and the analyte, respectively.

### Example 3-Real-Time Microarrays with Solid-Phase Melt Curve Analysis

The system for time-resolved optical detection is used to analyze a sample solution containing unknown amounts of analyte A and analyte B. In this experiment, an array of capturing probes A having donor reporter molecules at capturing spot A and capturing probes B having donor reporter molecules at capturing spot B are contacted with the sample solution containing unknown amounts of analytes A and analytes B. See FIG. 22A. The donor reporter on the probe has a longer fluorescence emission lifetime than that of the acceptor reporter on the analyte. Since there is sequence complementary between probe A and analyte A, and between probe B and analyte B, binding occurs between the probes A and B and the analytes A and B, respectively (FIG. 22A).

At the reset phase, the temperature in the reaction chamber is raised to 95° C. to dissociate any formed duplexes such that all probes A and B are free. Therefore, the measured signals from the capturing spots A and B reflects the highest amplitude and the longest emission lifetime corresponding to the donor reporter molecule.

At the capturing stage the temperature is lowered to and kept at 30° C. for a time period from 0 minute to t₁ minutes to allow the probes and the corresponding analytes to bind and form duplexes. Once the equilibrium is reached, the signals from the capturing spots A and B reflects the highest concentration of captured analytes A and B, respectively. At this time, the measured signals are a results from both free probes and duplexes, thereby displaying different profiles from those of the free probes only configurations.

At the denature stage, the temperature in the reaction chamber is then increased using the on-chip heaters, as shown in FIG. 22B, including time points of T=30° C. at t=t₁ minutes, T=95° C. at t=t₂ minutes. The device for time-resolved optical detection collects the emission signals from the capturing spot A and the capturing spot B and melt curves at both capturing spots are produced for the analytes A (black curve) and the analytes B (grey curve) relative to temperature (FIG. 22C). The capturing rates, and/or the time constraints, and/or the melting temperatures of the analytes A and the analytes B, respectively, are calculated based on the collected emission signals over time using the device for time-resolved optical detection, thereby allowing determining the concentrations of the analyte A and analyte B in the sample solution. The relative strength of binding for the two duplexes can be analyzed as well base on the two observed melt temperature.

### Example 4-Mutation Detection Using Differential Solid-Phase Melt Curve Analysis

An array of capturing probes A (5′-CCCTGAACTGGACCGT-3′) (SEQ ID NO: 1) having donor reporter molecules connected to the 5′-end at the capturing spot A and capturing probes B (5′-CCCTGAAGTGGACCGT-3′) (SEQ ID NO: 2) having donor reporter molecules connected to the 5′-end at the capturing spot B are contacted with a sample solution suspected to comprise either analytes A (5′-ACGGTCCAGTTCAGGG-3′, wild-type) (SEQ ID NO: 3) or analytes B (5′-ACGGTCCACTTCAGGG-3′, mutant) (SEQ ID NO: 4), each of which contain acceptor reporter molecules. The donor reporter molecule and the acceptor reporter molecule are a FRET pair. The donor reporter molecule has a longer fluorescence emission lifetime than that of the acceptor reporter molecule. The donor reporter molecule is Compound 4 and the acceptor reporter molecule is TMR. See, e.g., FIG. 23A. In other words, analyte A differs from analyte B by one nucleic acid, which are in the form of single nucleotide polymorphisms (SNPs).

In FIG. 23B, an example temperature profile for a real-time microarray plus differential melt curve analysis is shown in which the system is initially reset by elevating the temperature to 95° C. to denature all DNA-DNA duplexes that may have formed. Consequently, in the capturing phase of the mutation detection process the temperature in the reaction chamber is kept at 30° C. for a time period from 0 minute to t₁ minutes to allow the probes and the analytes to bind (hybridize) and form the duplexes. The temperature is then increased using the on-chip heaters, as shown in FIG. 23B, including time points of T=30° C. at t=0 minutes, T=30° C. at t=t₁ minutes, T=95° C. at t=t₂ minutes, followed by decreasing to T=30° C. beyond t=t₂ minutes. The device for time-resolved optical detection collects the emission signals in real-time time-resolved measurements from the capturing spot A and the capturing spot B on the array during the time intervals from t=0 minute to t=t₂ minute, during which the temperature of the reaction chamber is changed according to FIG. 23B. The respective melt curves are produced for signals received from the capturing spots A and B. The expected melt curve for the analytes A (wild type) captured at capturing spot A (black curve) and capturing spot B (grey curve) are shown in FIG. 23C; and the expected melt curve for the analytes B (mutant) captured at capturing spot A (black curve) and capturing spot B (grey curve) are shown in FIG. 23D. The capturing rates, and/or the time constraints, and/or the melting temperatures for the melt curves measured at the capturing spots A and B, respectively, are calculated based on the collected emission signals over time using the device for time-resolved optical detection. Accordingly, by comparing the recorded melt curves at capturing spots A and B with the melt curves at FIGS. 23C and 23D, one can determine whether the analyte is analyte A or analyte B, thereby determining the unknown analyte in the sample solution.

### Example 5-Solid-Phase Quantitative Polymerase Chain Reaction

A nucleic acid amplification testing (NAAT) system is built for real-time measurements using time-resolved optical reporters disclosed herein to conduct real-time, time-resolved, quantitative detection of newly generated amplification products (amplicons) over time. One of such NAAT system is shown in FIG. 24 for solid-phase quantitative PCR (SP-QPCR). Depending on how many different types of probes are immobilized in the reaction chamber, the NAAT system can monitor solution-phase PCR in single plex (i.e., one amplification in the reaction tube/chamber) or multiplex (i.e., multiple amplification processes in one reaction tube/chamber).

The immobilized probes at a specific independently addressable capturing spot contain the same donor reporter molecule. The probes have sequence complementary to one type of the amplicons (bearing either the forward primer or the reverse primer) formed during the amplification reaction. The targeted amplicon bears an acceptor reporter molecule. The donor reporter molecule and the acceptor reporter molecule are a FRET pair. The donor reporter molecule has a longer fluorescence emission lifetime than that of the acceptor reporter molecule. The donor reporter molecule is Compound 4 and the acceptor reporter molecule is TMR. When the acceptor reporter molecules are attached to the 5′-end of one of the primers, the probes are attached to the surface of the reaction chamber at their 5′-end while the donor reporter molecules are attached to the 3′-end.

Real-time, time-resolved quantification is carried out measuring the solid-phase hybridization of the amplicons bearing short lifetime fluorescence reporters (acceptors) at every PCR cycle and in the annealing and/or extension phase, and subsequently verifying the sequence of the generated amplicons at the completion of PCR process (end-point analysis) through a solid-phase melt curve analysis. The quantification relies on detecting the duplexes formed between the probes and the amplicons vs. the free probes and monitors the changes over time between these two groups.

References (all of which are incorporated by reference in their entirety)
1. Stimpson, D. I., Hoijer, J. V., Hsieh, W., Jou, C., Gordon, J., Theriault, T., Gamble, R. and Baldeschwieler, J. D., 1995. Real-time detection of DNA hybridization and melting on oligonucleotide arrays by using optical wave guides. Proceedings of the National Academy of Sciences, 92 (14), pp. 6379-6383.
2. Murib, M. S., Martens, D. and Bienstman, P., 2018. Label-free real-time optical monitoring of DNA hybridization using SiN Mach-Zehnder interferometer-based integrated biosensing platform. Journal of biomedical optics, 23 (12), p.127002.
3. Hassibi, A., Vikalo, H., Riechmann, J. L. and Hassibi, B., 2009. Real-time DNA microarray analysis. Nucleic acids research, 37 (20), pp. e132-e132.
4. Levine, P. M., Gong, P., Levicky, R. and Shepard, K. L., 2009. Real-time, multiplexed electrochemical DNA detection using an active complementary metal-oxide-semiconductor biosensor array with integrated sensor electronics. Biosensors and Bioelectronics, 24 (7), pp. 1995-2001.
5. Manickam, A., You, K. D., Wood, N., Pei, L., Liu, Y., Singh, R., Gamini, N., McDermott, M. W., Shahrjerdi, D., Kuimelis, R. G. and Hassibi, A., 2019. A CMOS Electrochemical Biochip With 32×32 Three-Electrode Voltammetry Pixels. IEEE Journal of Solid-State Circuits, 54 (11), pp. 2980-2990.
6. Hassibi, A., Manickam, A., Singh, R., Bolouki, S., Sinha, R., Jirage, K. B., McDermott, M. W., Hassibi, B., Vikalo, H., Mazarei, G. and Pei, L., 2018. Multiplexed identification, quantification and genotyping of infectious agents using a semiconductor biochip. Nature biotechnology, 36 (8), pp. 738-745.
7. Hassibi, A., Hassibi, B., Vikalo, H. and Riechmann, J. L., California Institute of Technology CalTech, 2015. Real time microarrays. U.S. Pat. No. 9,133,504.
8. Manickam, A., Singh, R., McDermott, M. W., Wood, N., Bolouki, S., Naraghi-Arani, P., Johnson, K. A., Kuimelis, R. G., Schoolnik, G. and Hassibi, A., 2017. A fully integrated CMOS fluorescence biochip for DNA and RNA testing. IEEE journal of solid-state circuits, 52 (11), pp. 2857-2870.
9. Xiao, Y., Lubin, A. A., Baker, B. R., Plaxco, K. W. and Heeger, A. J., 2006. Single-step electronic detection of femtomolar DNA by target-induced strand displacement in an electrode-bound duplex. Proceedings of the National Academy of Sciences, 103 (45), pp. 16677-16680.
10. Heeger, A. J., Fan, C. and Plaxco, K., 2011. Reagentless, reusable, bioelectronic detectors. U.S. Pat. No. 8,003,374.
11. Manickam, A., You, K. D., Wood, N., Pei, L., Liu, Y., Singh, R., Gamini, N., McDermott, M. W., Shahrjerdi, D., Kuimelis, R. G. and Hassibi, A., 2019. A CMOS Electrochemical Biochip With 32×32 Three-Electrode Voltammetry Pixels. IEEE Journal of Solid-State Circuits, 54 (11), pp. 2980-2990.
12. Selvin, P. R., 2003. Lanthanide-labeled DNA. In Topics in fluorescence spectroscopy (pp. 177-212). Springer, Boston, MA.
13. Creutz, C., Chou, M., Netzel, T. L., Okumura, M. and Sutin, N., 1980. Lifetimes, spectra, and quenching of the excited states of polypyridine complexes of iron (II), ruthenium (II), and osmium (II). Journal of the American Chemical Society, 102 (4), pp. 1309-1319.
14. Berezin, M. Y. and Achilefu, S., 2010. Fluorescence lifetime measurements and biological imaging. Chemical reviews, 110 (5), pp. 2641-2684.
15. Plaxco, K. W. and Soh, H. T., 2011. Switch-based biosensors: a new approach towards real-time, in vivo molecular detection. Trends in biotechnology, 29 (1), pp. 1-5.
16. Feagin, T. A., Maganzini, N. and Soh, H. T., 2018. Strategies for creating structure-switching aptamers. ACS sensors, 3 (9), pp. 1611-1615.
17. Hsieh, K., White, R. J., Ferguson, B. S., Plaxco, K. W., Xiao, Y. and Soh, H. T., 2011. Polarity-switching electrochemical sensor for specific detection of single-nucleotide mismatches. Angewandte Chemie International Edition, 50 (47), pp. 11176-11180.
18. Hassibi, A., California Institute of Technology CalTech, 2014. Integrated semiconductor bioarray. U.S. Pat. No. 8,637,436.
19. Hassibi, A., Singh, R. and Manickam, A., University of Texas System, 2015. Integrated optical biosensor array including charge injection circuit and quantizer circuit. U.S. Pat. No. 8,969,781.
20. Hassibi, A., Manickam, A., Singh, R. and Kuimelis, R. G., InSilixa Inc, 2020. Methods and systems for time-gated fluorescent-based detection. U.S. Pat. No. 11,360,029.
21. Vikalo, H., Hassibi, B. and Hassibi, A., 2008. Modeling and estimation for real-time microarrays. IEEE journal of selected topics in signal processing, 2 (3), pp. 286-296.
22. Farrar, J. S. and Wittwer, C. T., 2017. High-resolution melting curve analysis for molecular diagnostics. In Molecular diagnostics (pp. 79-102). Academic Press.
23. Hassibi, A., Jirage, K., Manickam, A. and Milaninia, K., InSilixa Inc, 2017. Multiplexed analysis of nucleic acid hybridization thermodynamics using integrated arrays. U.S. Pat. No. 9,708,647.
24. Clutter, D. S., Mazarei, G., Sinha, R., Manasa, J., Nouhin, J., LaPrade, E., Bolouki, S., Tzou, P. L., Hannita-Hui, J., Sahoo, M. K. and Kuimelis, P., 2019. Multiplex solid-phase melt curve analysis for the point-of-care detection of HIV-1 drug resistance. The Journal of Molecular Diagnostics, 21 (4), pp. 580-592.
25. Hassibi, A., Ebert, J., Bolouki, S., Anemogiannis, A., Mazarei, G., Li, Y., Johnson, K. A., Van, T., Mantina, P., Gharooni, T. and Jirage, K., 2018. An array-based melt curve analysis method for the identification and classification of closely related pathogen strains. Biology Methods and Protocols, 3 (1), p.bpy005.
26. Mullis, K. B., 1994. The polymerase chain reaction (Vol. 41, No. 5). Springer science & business media.
27. Wiedmann, M., Wilson, W. J., Czajka, J., Luo, J., Barany, F. and Batt, C. A., 1994. Ligase chain reaction (LCR)—overview and applications. PCR Methods Appl, 3 (4), pp.S51-64.
28. Parida, M., Sannarangaiah, S., Dash, P. K., Rao, P. V. L. and Morita, K., 2008. Loop mediated isothermal amplification (LAMP): a new generation of innovative gene amplification technique; perspectives in clinical diagnosis of infectious diseases. Reviews in medical virology, 18 (6), pp. 407-421.
29. Wang, Y., Sun, H., Xu, G., Guan, M., Zhang, Q., Wang, Z., Dong, Z., Chen, W., Yang, X., Qiao, A. and Fan, Y., 2022. A multiplexed electrochemical quantitative polymerase chain reaction platform for single-base mutation analysis. Biosensors and Bioelectronics, 214, p.114496.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-113. (canceled)

114. A real-time detection system for at least one analyte, comprising:
(a) a reaction chamber comprising a plurality of capturing probes immobilized at an independently addressable location on a surface of said reaction chamber, wherein each of said plurality of capturing probes comprises at least one reporter molecule;
(b) a sensor array comprising a sensor in optical communication with said independently addressable location on said surface, wherein said sensor is configured to detect at least one time-resolved signal from said independently addressable location in real-time; and
(c) a light source configured to synchronize with said sensor array and emit a pulse of excitation energy when turned on, wherein said at least one time-resolved signal is collected after said light source is turned off,
wherein said at least one reporter molecule is a fluorescence reporter, wherein, when a capturing probe comprising said fluorescence reporter is not bound with an analyte, said fluorescence reporter is configured to emit a first fluorescence comprising a first time-resolved signal with a first fluorescence emission lifetime upon excitation by said pulse of excitation energy,
wherein, when said capturing probe is bound with said analyte, said fluorescence reporter is configured to emit a second time-resolved signal with a second fluorescence emission lifetime upon excitation by said pulse of excitation energy, and wherein said second fluorescence emission lifetime is different from said first fluorescence emission lifetime.

115. The real-time detection system of claim 114, wherein said fluorescence reporter is an energy donor and forms a donor-quencher pair with a non-radiating quencher, wherein, when said fluorescence reporter is brought into a proximity of said non-radiating quencher, said first fluorescence is quenched by said non-radiating quencher.

116. The real-time detection system of claim 115, wherein said analyte comprises said non-radiating quencher, wherein said capturing probe is configured to capture said analyte and form a duplex, wherein said duplex brings said energy donor and said non-radiating quencher into said proximity.

117. The real-time detection system of claim 114, wherein said fluorescence reporter is a donor of a fluorescence resonance energy transfer (FRET) system, wherein, when said capturing probe is not bound with said analyte, said fluorescence reporter is configured to emit said first fluorescence.

118. The real-time detection system of claim 117, wherein said FRET system comprises an acceptor, wherein, when said acceptor is brought within a vicinity of said donor to form said FRET system, said acceptor is configured to emit said second time-resolved signal.

119. The real-time detection system of claim 118, wherein said analyte comprises said acceptor.

120. The real-time detection system of claim 119, wherein said capturing probe is configured to capture said analyte and form a duplex, wherein said duplex brings said donor and said acceptor into said vicinity.

121. The real-time detection system of claim 119, wherein said capturing probe is configured to capture said analyte and form a duplex, wherein said duplex intercalates an intercalator comprising said acceptor.

122. The real-time detection system of claim 121, wherein said duplex brings said donor and said acceptor into said vicinity.

123. The real-time detection system of claim 114, wherein said at least one reporter molecule comprises a fluorescence resonance energy transfer (FRET) system comprising a donor and an acceptor, wherein said donor is configured to emit said first fluorescence, wherein, when said acceptor is brought within a vicinity of said donor to form said FRET system, said acceptor is configured to emit said second time-resolved signal.

124. The real-time detection system of claim 123, wherein, when said capturing probe is bound with said analyte, said capturing probe is in a first configuration which emits said first time-resolved signal.

125. The real-time detection system of claim 124, wherein, when said capturing probe is not bound with said analyte, said capturing probe is in a second configuration which emits said second time-resolved signal.

126. The real-time detection system of claim 114, wherein said at least one reporter molecule comprises an energy donor and a non-radiating quencher, wherein said energy donor is configured to emit said first fluorescence, wherein said first fluorescence is quenched by said non-radiating quencher when said energy donor is brought into a proximity of said non-radiating quencher.

127. The real-time detection system of claim 126, wherein, when said capturing probe is not bound with said analyte, said capturing probe is in a first configuration which brings said energy donor and said non-radiating quencher into said proximity.

128. The real-time detection system of claim 127, wherein, when said capturing probe is bound with said analyte, said capturing probe is in a second configuration which emits said second time-resolved signal.

129. The real-time detection system of claim 114, wherein a ratio of an overall error/inaccuracy of timing measurement to said first fluorescence emission lifetime or said second fluorescence emission lifetime is no more than 0.01.

130. The real-time detection system of claim 114, further comprising a focal plane imager with an electrical shutter, wherein said focal plane imager is configured to take time-gated images of said surface.

131. The real-time detection system of claim 130, wherein said focal plane imager is a camera or a microscope.

132. The real-time detection system of claim 131, further comprising at least one control probe immobilized on said surface, wherein said at least one control probe is:
(i) a donor control probe comprising a fluorescence donor, wherein said donor control probe is configured not to capture or interact with said analyte; or
(ii) an acceptor control probe comprising a fluorescence resonance energy transfer (FRET) control system comprising a FRET donor and a FRET acceptor, wherein said FRET control system is configured to emit a FRET signal from said FRET acceptor, wherein said acceptor control probe is configured not to capture or interact with said analyte; or
(iii) a blank probe configured not to emit a fluorescence signal; or
(iv) a combination thereof.

133. The real-time detection system of claim 114, wherein said sensor comprises:
(i) a first optical transducer in optical communication with said surface;
(ii) a second optical transducer disposed adjacent to said first optical transducer; and
(iii) an optical cover disposed over said second optical transducer,
wherein said first optical transducer is configured to collect said first time-resolved signal and said second time-resolved signal.


SEQUENCE LISTING (4 sequences)

SEQ ID NO: 1 - DNA, 16 bp, synthetic construct
  source 1..16: mol_type = other DNA
ccctgaactggaccgt

SEQ ID NO: 2 - DNA, 16 bp, synthetic construct
  source 1..16: mol_type = other DNA
ccctgaagtggaccgt

SEQ ID NO: 3 - DNA, 16 bp, synthetic construct
  source 1..16: mol_type = other DNA
acggtccagttcaggg

SEQ ID NO: 4 - DNA, 16 bp, synthetic construct
  source 1..16: mol_type = other DNA
acggtccacttcaggg